(19) United States
(12) Patent Application Publication
(10) Pub. No.: US 2022/0202792 A1
(43) Pub. Date: Jun. 30, 2022

(54) METHODS AND COMPOSITIONS FOR TREATING CHRONIC INFLAMMATORY INJURY, METAPLASIA, DYSPLASIA AND CANCERS OF EPITHELIAL TISSUES

(71) Applicants: University of Houston System, Houston, TX (US); The Board of Rgents of The University of Texas System, Austin, TX (US); Tract Pharmaceuticals, Inc., West Hartford, CT (US)

(72) Inventors: Frank McKEON, Sugar Land, TX (US); Marcin DULEBA, Houston, TX (US); Yanting ZHANG, Houston, TX (US); Jingzhong XIE, Houston, TX (US); Wa XIAN, Sugar Land, TX (US); Matthew VINCENT, Amesbury, MA (US)

(73) Assignees: University of Houston System, Houston, TX (US); The Board of Regents of The University of Texas System, Austin, TX (US); Tract Pharmaceuticals, Inc., West Hartford, CT (US)

(21) Appl. No.: 17/606,445

(22) PCT Filed: Apr. 24, 2020
(86) PCT No.: PCT/US2020/029933

§ 371 Date: Oct. 25, 2021

Related U.S. Application Data
(60) Provisional application No. 62/839,152, filed on Apr. 26, 2019.
(60) Provisional application No. 62/924,978, filed on Oct. 23, 2019.

(51) Int. Cl. A61K 31/444; A61K 9/00; A61K 45/06; A61K 31/69; A61K 31/675; A61K 31/5377; A61K 31/506; A61K 31/381; A61K 31/351; A61K 31/496; A61K 31/5025; A61K 31/553; A61K 31/4709; A61K 31/44; A61K 31/404; A61K 31/517; A61K 9/14; A61P 35/00

(52) U.S. Cl. CPC A61K 31/444; A61P 35/00; A61K 45/06; A61K 31/69; A61K 31/675; A61K 31/5377; A61K 31/506; A61K 31/381; A61K 31/351; A61K 31/496; A61K 31/5025; A61K 31/553; A61K 31/4709; A61K 31/44; A61K 31/404; A61K 31/517; A61K 9/14; A61K 9/0053; A61K 9/0024

(57) ABSTRACT

The present disclosure provides methods and formulations for treating a patient suffering from one or more of chronic inflammatory injury, metaplasia, dysplasia or cancer of an epithelial tissue, which method comprises administering to the patient an agent that selectively kills or inhibits the proliferation or differentiation of pathogenic epithelial stem cells (PESCs) relative to normal epithelial stem cells in the tissue in which the PESCs are found. Representative epithelial tissues include pulmonary, genitourinary, gastrointestinal/esophageal, pancreatic and hepatic tissues.

### PRIORITY CLAIM

The present application claims benefit of priority to U.S. Provisional Applications Ser. Nos. 62/839,152 and 62/924,978, filed Apr. 26, 2019 and Oct. 23, 2019, respectively, the entire contents of each application being incorporated herein by reference.

### FEDERAL FUNDING DISCLOSURE

The invention was made with government support under Grant No. U24CA228550 awarded by the National Institutes of Health. The government has certain rights in the invention.

### BACKGROUND

Metaplasia is the replacement of one differentiated cell type with another mature differentiated cell type that is not normally present in a specific tissue. Typically, metaplasia is triggered by environmental stimuli, which may act in concert with the deleterious effects of microorganisms and inflammation. A hallmark of metaplasia is a change in cellular identity. Universally, metaplasia is a precursor to low-grade dysplasia, which can culminate in high-grade dysplasia and carcinoma. See FIG. 8. Typically, the risk of a patient developing cancer increases in a pronounced manner as an inflammatory disease or metaplasia progresses to dysplasia.

FIG. 9 provides a statistical overview of the risk associated with Barret's Esophagus (BE). BE is the result of chronic gastroesophageal reflux disease (GERD) and represents the end stage of the natural course of this disease. It has been estimated that 20% of the population in the United States suffers from gastroesophageal reflux and that about 10% of these patients are diagnosed with BE. Commonly, BE is discovered during endoscopy for the evaluation of GERD symptoms.

It is documented that longstanding exposure of esophageal mucosa to gastric acidity results in cellular damage of the stratified squamous epithelium and creates an abnormal environment, which stimulates repair in the form of intestinal epithelial metaplasia. The consequence is that the stratified squamous epithelium, which physiologically lines the esophageal mucosa, is replaced by a pathological, specialized columnar epithelium which is neither of cardiac nor of stomach type, but exhibits features of the intestinal type of epithelium. This pathological type of epithelium usually demonstrates DNA alterations that predispose to malignancy. The alterations in BE are histologically classified into three categories, depending on whether or not they exhibit dysplasia: (1) BE without dysplasia; (2) BE with low-grade dysplasia; and (3) BE with high-grade dysplasia (HGD). In BE with HGD, dysplasia is confined to the mucosa without crossing the basement membrane. If dysplasia extends beyond the basement membrane into the lamina propria through the in-coming lymphatic network, it is defined as intramucosal (superficial) adenocarcinoma, whereas if it invades the muscularis mucosa layer it becomes invasive adenocarcinoma. Thus, BE with HGD is considered a precursor of invasive adenocarcinoma.

Six to twenty percent of patients with BE and HGD are at greatest risk of developing adenocarcinoma within a short period of time, ranging from 17 to 35 month at follow-up. Esophagectomy specimens from patients with BE and HGD revealed invasive adenocarcinoma in 30%-40% of cases. A recent meta-analysis demonstrated that patients with BE and HGD developed esophageal adenocarcinoma with an average incidence of 6 every 100 patients per year, during the first 1.5 to 7 years of endoscopic surveillance. Furthermore, the majority of esophageal adenocarcinoma is thought to have evolved from cells that have undergone Barrett's metaplasia.

BE is also classified into two categories according to the extent of intestinal metaplasia above the gastroesophageal junction: (1) long segment BE, if the extent of the intestinal epithelium is greater than 3 cm; and (2) short segment BE, if it is less than 3 cm. Among patients who undergo endoscopy for symptoms of GERD, the incidence of long segment BE is 3%-5%, whereas short segment BE occurs in 10%-15%. Whether long and short segment BE share the same pathogenetic alterations or the same predisposition to malignancy still remains unclear; however, both conditions are currently treated in the same manner.

A common, and invasive, means for treating certain Barrett's Esophagus patients is through endoscopic ablation therapy, such as radiofrequency ablation, photodynamic therapy or cryoablation of esophageal tissue. However, despite a reasonably high percentage of patients that reach remission after therapy, many of those patients relapse within a few years. For other patients, whether because they are refractory to ablative therapy or ineligible due to severe co-morbidities, there are even fewer treatment options and those that exist still leave a significant need for more effective therapies with better results and/or long durations of remission.

Similar metaplasia-to-dysplasia-to-cancer transitions are observed across a variety of other epithelial tissues. Metaplasia tends to occur in tissues constantly exposed to environmental agents, which are often injurious in nature. For example, the pulmonary system (lungs and trachea) and the gastrointestinal tract are common sites of metaplasia owing to their contacts with air and food, respectively. In the ovaries, the dynamic interaction between ovarian surface epithelium and underlying ovarian stroma appears to be the origin of epithelial differentiation, metaplasia and finally malignant transformation.

There is a substantial unmet medical need not only for treatments that are effective for cancers of epithelial tissues, but also treatments directed to metaplasia and dysplasia of those tissues.

### SUMMARY

One aspect of the present disclosure provides a method for treating a patient suffering from chronic inflammatory injury, metaplasia, dysplasia or cancer of an epithelial tissue, which method comprises administering to the patient an anti-PESC agent that selectively kills or inhibits the proliferation or differentiation of pathogenic epithelial stem cells (PESCs) relative to normal epithelial stem cells in the tissue in which the PESC is found. Representative epithelial tissues include pulmonary, genitourinary, gastrointestinal, pancreatic and hepatic tissues.

Another aspect of the disclosure provides a method of reducing proliferation, survival, migration, or colony formation ability of PESCs in a subject in need thereof comprising contacting the PESC with a therapeutically effective amount of an anti-PESC agent that selectively kills or inhibits the proliferation or differentiation of a PESC population relative to normal epithelial stem cells in the tissue in which the PESCs are found.

Another aspect of the disclosure provides a pharmaceutical preparation for treating one or more of chronic inflammatory injury, metaplasia, dysplasia or cancer of an epithelial tissue, which preparation comprises an anti-PESC agent that selectively kills or inhibits the proliferation or differentiation of PESCs relative to normal epithelial stem cells. In certain embodiments, the target tissue for treatment is lung. In certain embodiments, the target tissue for treatment is a lung tumor, such as for the treatment of non-small cell lung carcinoma (NSCLC) or small cell lung carcinoma (SCLC). In certain embodiments, the target tissue for treatment is an ovarian, fallopian and/or cervical tissue, such as for the treatment of cervical metaplasia, cervical cancer, fallopian cancer and/or ovarian cancer (including taxol and/or cisplatin-resistant ovarian cancer).

For example, the present disclosure provides a method for treating a patient suffering from one or more of esophagitis (including Eosinophilic esophagitis or EoE), Barrett's Esophagus, esophageal dysplasia or esophageal cancer, which method comprises administering to the patient an anti-PESC agent that selectively kills or inhibits the proliferation or differentiation of Barrett's Esophagus stem cells (BESC) relative to normal esophageal stem cells. In certain embodiments, the patient presents with esophagitis. In certain embodiments, the patient presents with Barrett's Esophagus. In certain embodiments, the patient presents with esophageal dysplasia. In certain embodiments, the patient presents with esophageal cancer. In certain embodiments, the patient presents with esophageal carcinoma, such as esophageal adenocarcinoma or esophageal squamous cell carcinoma.

Another aspect of the disclosure provides a method of reducing proliferation, survival, migration, or colony formation ability of a BESC in a subject in need thereof comprising contacting the BESC with a therapeutically effective amount of an anti-PESC agent that selectively kills or inhibits the proliferation or differentiation of BESC relative to normal esophageal stem cells.

Another aspect of the disclosure provides a pharmaceutical preparation for treating one or more of esophagitis, Barrett's esophagus, esophageal dysplasia or esophageal cancer, which preparation comprises an anti-PESC agent that selectively kills or inhibits the proliferation or differentiation of BESCs relative to normal esophageal stem cells. In certain embodiments, the patient presents with esophagitis. In certain embodiments, the patient presents with Barrett's Esophagus. In certain embodiments, the patient presents with esophageal dysplasia. In certain embodiments, the patient presents with esophageal cancer. In certain embodiments, the patient presents with esophageal carcinoma, such as esophageal adenocarcinoma or esophageal squamous cell carcinoma.

Yet another aspect of the disclosure provides a drug eluting device, such as for treating one or more of esophagitis, Barrett's esophagus, esophageal dysplasia or esophageal cancer, which device comprises drug release means including an anti-PESC agent that selectively kills or inhibits the proliferation or differentiation of PESCs relative to normal epithelial stem cells, which device when deployed in a patient positions the drug release means proximal to the luminal surface of the esophagus and releases the agent in an amount sufficient to achieve a therapeutically effective exposure of the luminal surface to the agent. In certain embodiments, the patient presents with esophagitis. In certain embodiments, the patient presents with Barrett's Esophagus. In certain embodiments, the patient presents with esophageal dysplasia. In certain embodiments, the patient presents with esophageal cancer. In certain embodiments, the patient presents with esophageal carcinoma, such as esophageal adenocarcinoma or esophageal squamous cell carcinoma. Examples of drug eluting devices are drug eluting stents, drug eluting collars and drug eluting ballons.

In other embodiments, there are provided drug eluting devices that can be implanted proximal to the diseased portion of the luminal surface of the esophagus, such as implanted extraluminally (i.e., submucosally or in or on the circular muscle or longitudinal muscle) rather than intraluminally.

In certain embodiments, the anti-PESC agent has an IC₅₀ for selectively killing PESCs that is ⅕^{th} or less the IC₅₀ for killing normal epithelial stem cells in the tissue in which the PESCs are found, more preferably 1/10^{th}, 1/20^{th}, 1/50^{th}, 1/100th, 1/250^{th}, 1/500^{th} or even 1/1000^{th} or less the IC₅₀ for killing normal epithelial stem cells.

In certain embodiments, the anti-PESC agent has an IC₅₀ for selectively killing BESCs that is ⅕^{th} or less the IC₅₀ for killing normal esophageal stem cells, more preferably 1/10^{th}, 1/20^{th}, 1/50^{th}, 1/100th, 1/250^{th}, 1/500^{th} or even 1/1000^{th} or less the IC₅₀ for killing normal esophageal stem cells.

In certain embodiments, the anti-PESC agent has an IC₅₀ for selectively inhibiting the proliferation of PESCs that is ⅕^{th} or less the IC₅₀ for inhibiting normal epithelial stem cells in the tissue in which the PESCs are found, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal epithelial stem cells.

In certain embodiments, the anti-PESC agent has an IC₅₀ for selectively inhibiting the proliferation of BESCs that is ⅕^{th} or less the IC₅₀ for inhibiting the proliferation of normal esophageal stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal esophageal stem cells.

In certain embodiments, the anti-PESC agent has an IC₅₀ for selectively inhibiting the differentiation of PESCs that is ⅕^{th} or less the IC₅₀ for inhibiting the differentiation of normal epithelial stem cells, more preferably 1/10^{th}, 1/20^{th}, 1/50^{th}, 1/100th, 1/250^{th}, 1/500^{th} or even 1/1000^{th} or less the IC₅₀ for inhibiting the differentiation of normal epithelial stem cells.

In certain embodiments, the anti-PESC agent has an IC₅₀ for selectively inhibiting the differentiation of BESCs that is ⅕^{th} or less the IC₅₀ for inhibiting the differentiation of normal esophageal stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the differentiation of normal esophageal stem cells.

In certain embodiments, the anti-PESC agent has a therapeutic index (TI) for treating esophagitis, Barrett's Esophagus, esophageal dysplasia and/or esophageal cancer of at least 2, and more preferably has a therapeutic index of at least 5, 10, 20, 50, 100, 250, 500 or 1000 for treating esophagitis, Barrett's Esophagus, esophageal dysplasia and/or esophageal cancer.

In certain embodiments, the anti-PESC agent has a therapeutic index (TI) for treating ovarian, fallopian and or cervical metaplasia or dysplasia of at least 2, and more preferably has a therapeutic index of at least 5, 10, 20, 50, 100, 250, 500 or 1000.

In certain embodiments, the anti-PESC agent has a therapeutic index (TI) for treating ovarian cancer (such as taxol and/or cisplatin resistant ovarian cancer) of at least 2, and more preferably has a therapeutic index of at least 5, 10, 20, 50, 100, 250, 500 or 1000.

In certain embodiments, the anti-PESC agent has a therapeutic index (TI) for treating lung cancer (such NSCLC or SCLC) of at least 2, and more preferably has a therapeutic index of at least 5, 10, 20, 50, 100, 250, 500 or 1000.

In certain embodiments, the anti-PESC agent has a therapeutic index (TI) for treating lung metaplasia or dysplasia of at least 2, and more preferably has a therapeutic index of at least 5, 10, 20, 50, 100, 250, 500 or 1000.

In certain embodiments, the anti-PESC agent inhibits the proliferation or differentiation of PESCs, or kills PESCs, with an IC₅₀ of 10⁻⁶ M or less, more preferably 10⁻⁷ M or less, 10⁸ M or less or 10⁻⁹ M or less.

In certain embodiments, the anti-PESC agent inhibits the proliferation or differentiation of BESCs, or kills BESCs, with an IC₅₀ of 10⁻⁶ M or less, more preferably 10⁻⁷ M or less, 10⁻⁸ M or less or 10⁻⁹ M or less.

In certain embodiments, the anti-PESC agent is administered during or after endoscopic ablation therapy, such as radiofrequency ablation, photodynamic therapy or cryoablation of esophageal tissue.

In certain embodiments, the anti-PESC agent is administered by topical application, such as to esophageal tissue, genitourinary tissue or lung tissue.

In certain embodiments, the anti-PESC agent is administered by submucosal injection, such as to esophageal tissue, genitourinary tissue or lung tissue.

In certain embodiments, the anti-PESC agent is formulated for submucosal injection, such as to esophageal tissue, genitourinary tissue or lung tissue.

In certain embodiments, the anti-PESC agent is formulated for topical application, such as to esophageal tissue, genitourinary tissue or lung tissue.

In certain embodiments, the anti-PESC agent is formulated as part of a bioadhesive formulation.

In certain embodiments, the anti-PESC agent is formulated as part of a drug-eluting particle, drug eluting matrix or drug-eluting gel.

In certain embodiments, the anti-PESC agent is formulated as part of a bioerodible drug-eluting particle, bioerodible drug eluting matrix or bioerodible drug-eluting gel.

In certain embodiments, the disclosure provides a esophageal topical retentive formulation for topical application to the luminal surface of the esophagus, comprising (i) an anti-PESC agent that selectively kills or inhibits the proliferation or differentiation of pathogenic epithelial stem cells relative to normal esophageal stem cells, (ii) a bioadhesive, and (iii) optionally, one or more pharmaceutically acceptable excipients.

For instance, the formulation can have a mucosal surface residence half-life on esophageal tissue of at least 30 minutes, more preferably at least 60, 120, 180, 240 or even 300 minutes.

For instance, the formulation can produce at least a minimally effective concentration (MEC) of the anti-PESC agent in the esophageal tissue to which it is applied to which it is applied for at least 30 minutes, more preferably at least 60, 120, 180, 240 or even 300 minutes.

For instance, the formulation can produce anti-PESC agent concentration in the esophageal tissue to which it is applied with T1/2 of at least 2 hours, more preferably at least 4, 6, 8, 10 or even 12 hours.

In certain embodiments, the formulation produces a systemic concentration of the anti-PESC agent which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In certain embodiments, the topical formulation is a viscous bioadhesive liquid to coat the esophagus.

In certain embodiments, the topical formulation comprises anti-PESC eluting multiparticulates, microparticles, nanoparticles or microdiscs

In further embodiments, there is provided bioadhesive nanoparticle having a polymeric surface with an adhesive force equivalent to an adhesive force of between 10 N/m² and 100,000 N/m² measured on human mucosal surfaces, which nanoparticle further includes at least one anti-PESC agent, the anti-PESC agent dispersed therein or thereon, wherein the nanoparticle elutes the anti-PESC agents into the mucous gel layer when adhered to mucosal tissue.

The anti-PESC agent(s) may be selected from, for example, a histone demethylase inhibitor, A JmjC demethylase inhibitors, a pan-JmjC demethylase inhibitors, a receptor tyrosine kinase inhibitor, an EGFR inhibitor, a HER2 inhibitor, a dual EGFR/HER2 inhibitor, a proteasome inhibitor, an immunoproteasome inhibitor, a STAT inhibitor, a STAT3 inhibitor, a FLT3 inhibitor, a GSK3 inhibitor, an HSP90 inhibitor, an HSP70 inhibitor and a dual HSP90/HSP70 inhibitor, or a combination thereof,

In certain embodiments, the bioadhesive nanoparticle further includes at least one ESO Regenerative agent dispersed therein or thereon, wherein the nanoparticle elutes the both the anti-PESC agent and ESO Regenerative agent into the mucous gel layer when adhered to mucosal tissue.

In certain embodiments, the ESO Regenerative agent is pan-inhibitor of ABL kinase inhibitor, preferably a BCR-ABL kinase inhibitor. Exemplary pan-inhibitor include imatinib, nilotinib, dasatinib, bosutinib and ponatinib, and is preferably ponatinib.

In certain embodiments, the ESO Regenerative agent is a BACE inhibitor, an FAK inhibitor, a VEGR inhibitor or an AKT inhibitor.

In certain embodiments, the submucosal retentive formulation produces a systemic concentration of the ESO Regenerative Agent, such as ponatinib, which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In still other embodiments, there is provided a submucosal retentive formulation comprising at least one anti-PESC agent and one or more pharmaceutically acceptable excipients, which formulation is injectable submucosally and forms a submucusal depot releasing an effective amount of the anti-PESC agent into the surrounding tissue.

In certain embodiments, the submucosal retentive formulation is an injectable thermogel for submucosal injection, comprising at least one anti-PESC agent and one or more pharmaceutically acceptable excipients, wherein the thermogel has a low-viscosity fluid at room temperature (and easily injected), and becomes a non-flowing gel at body temperature after injection.

To illustrate, the anti-PESC agent(s) formulated in the submucosal retentive formulations may be selected from, for example, a histone demethylase inhibitor, A JmjC demethylase inhibitors, a pan-JmjC demethylase inhibitors, a receptor tyrosine kinase inhibitor, an EGFR inhibitor, a HER2 inhibitor, a dual EGFR/HER2 inhibitor, a proteasome inhibitor, an immunoproteasome inhibitor, a STAT inhibitor, a STAT3 inhibitor, a FLT3 inhibitor, a GSK3 inhibitor, an HSP90 inhibitor, an HSP70 inhibitor and a dual HSP90/HSP70 inhibitor, or a combination thereof,

In certain embodiments, the submucosal retentive formulations further includes at least one ESO Regenerative agent dispersed therein, wherein the submucosal retentive formulations release the both the anti-PESC agent and ESO Regenerative agent into the tissue surrounding the site of submucosal injection.

In certain embodiments, the ESO Regenerative agent is pan-inhibitor of ABL kinase inhibitor, preferably a BCR-ABL kinase inhibitor. Exemplary pan-inhibitor include imatinib, nilotinib, dasatinib, bosutinib and ponatinib, and is preferably ponatinib.

In certain embodiments, the ESO Regenerative agent is a BACE inhibitor, an FAK inhibitor, a VEGR inhibitor or an AKT inhibitor.

For instance, the submucosal retentive formulation can have a submucosal residence half-life in esophageal tissue of at least 30 minutes, more preferably at least 60, 120, 180, 240 or even 300 minutes.

For instance, the submucosal retentive formulation can produce at least a minimally effective concentration (MEC) of the anti-PESC agent in the esophageal tissue in which it is injected for at least 30 minutes, more preferably at least 60, 120, 180, 240 or even 300 minutes.

For instance, the submucosal retentive formulation can produce anti-PESC agent concentration in esophageal tissue in which it is injected with T1/2 of at least 2 hours, more preferably at least 4, 6, 8, 10 or even 12 hours.

The present disclosure also provides submucosal retentive formulations of ESO Regenerative Agents. For example the formulation can include (i) a BCR-ABL kinase inhibitor, and (ii) one or more pharmaceutically acceptable excipients, which formulation is injectable submucosally and forms a submucusal depot releasing an effective amount of the BCR-ABL kinase inhibitor to the surrounding tissue. In certain preferred embodiments, the BCR-ABL kinase inhibitor is ponatinib. In certain preferred embodiments, the BCR-ABL kinase inhibitor is a FLT3 inhibitor such as quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

In certain embodiments, the submucosal retentive formulation can have a submucosal residence half-life in esophageal tissue of at least 30 minutes, more preferably at least 60, 120, 180, 240 or even 300 minutes.

In certain embodiments, the submucosal retentive formulation can produce at least a minimally effective concentration (MEC) of the ESO Regenerative Agent in the esophageal tissue in which it is injected for at least 30 minutes, more preferably at least 60, 120, 180, 240 or even 300 minutes.

In certain embodiments—the submucosal retentive formulation can produce an ESO Regenerative Agent concentration in esophageal tissue in which it is injected with T1/2 of at least 2 hours, more preferably at least 4, 6, 8, 10 or even 12 hours.

In certain embodiments, the submucosal retentive formulation produces a systemic concentration of the ESO Regenerative Agent, such as ponatinib, which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In each of the above submucosal retentive formulations, the formulation can form a flowable and/or viscous gel.

In certain embodiments, the formulation is an injectable thermogel. Thermogels includes, merely to illustrate, poly(lactic acid-co-glycolic acid)-poly(ethylene glycol)-poly(lactic acid-co-glycolic acid) (PLGA-PEG-PLGA) triblock copolymers.

In certain embodiments, the formulation is a hydrogel.

In certain embodiments, the formulation is suitable for endoscopic dissection.

In certain embodiments, the formulation further comprises an anticoagulant.

In certain embodiments, the formulation further comprises comprises one or more antitussives, antihistamines, antipyretics, analgesics, anti-infective agents and/or chemotherapeutic agents

Another aspect of the present disclosure provides an injectable thermogel for submucosal injection, comprising ponatinib and (optionally) one or more pharmaceutically acceptable excipients, wherein the thermogel has a low-viscosity fluid at room temperature (and easily injected), and becomes a non-flowing gel at body temperature after injection.

In certain embodiments, the disclosure provides an esophageal topical retentive formulation for topical application to the luminal surface of the esophagus, comprising (i) an ESO Regenerative Agent, (ii) a bioadhesive, and (iii) optionally, one or more pharmaceutically acceptable excipients. For example the formulation can include (i) a BCR-ABL kinase inhibitor, and (ii) one or more pharmaceutically acceptable excipients, which formulation is injectable submucosally and forms a submucusal depot releasing an effective amount of the BCR-ABL kinase inhibitor to the surrounding tissue. In certain preferred embodiments, the BCR-ABL kinase inhibitor is ponatinib. In certain preferred embodiments, the BCR-ABL kinase inhibitor is a FLT3 inhibitor such as quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

For instance, the topical formulation can have a mucosal surface residence half-life on esophageal tissue of at least 30 minutes, more preferably at least 60, 120, 180, 240 or even 300 minutes.

For instance, the topical formulation can produce at least a minimally effective concentration (MEC) of the ESO Regenerative Agent in the esophageal tissue to which it is applied to which it is applied for at least 30 minutes, more preferably at least 60, 120, 180, 240 or even 300 minutes.

For instance, the topical formulation can produce ESO Regenerative Agent concentration in the esophageal tissue to which it is applied with T1/2 of at least 2 hours, more preferably at least 4, 6, 8, 10 or even 12 hours.

In certain embodiments, the submucosal retentive formulation produces a systemic concentration of the ESO Regenerative Agent, such as ponatinib, which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In certain embodiments, the topical formulation is a viscous bioadhesive liquid to coat the esophagus.

In certain embodiments, the topical formulation comprises anti-PESC eluting multiparticulates, microparticles, nanoparticles or microdiscs

In certain embodiments, the topical formulation further comprises an anticoagulant.

In certain embodiments, the topical formulation further comprises comprises one or more antitussives, antihistamines, antipyretics, analgesics, anti-infective agents and/or chemotherapeutic agents

In further embodiments, there is provided bioadhesive nanoparticle having a polymeric surface with an adhesive force equivalent to an adhesive force of between 10 N/m² and 100,000 N/m² measured on human mucosal surfaces, which nanoparticle further includes at least one ESO Regenerative Agent, the ESO Regenerative Agent dispersed therein or thereon, wherein the nanoparticle elutes the ESO Regenerative Agent into the mucous gel layer when adhered to mucosal tissue. For example the formulation can include (i) a BCR-ABL kinase inhibitor, and (ii) one or more pharmaceutically acceptable excipients, which formulation is injectable submucosally and forms a submucusal depot releasing an effective amount of the BCR-ABL kinase inhibitor to the surrounding tissue. In certain preferred embodiments, the BCR-ABL kinase inhibitor is ponatinib. In certain preferred embodiments, the BCR-ABL kinase inhibitor is a FLT3 inhibitor such as quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

In certain embodiments, the submucosal retentive formulation produces a systemic concentration of the ESO Regenerative Agent, such as ponatinib, which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In certain embodiments, the bioadhesive nanoparticle further comprises an anticoagulant.

In certain embodiments, the bioadhesive nanoparticle further comprises one or more antitussives, antihistamines, antipyretics, analgesics, anti-infective agents and/or chemotherapeutic agents

In further embodiments, there is provided a drug eluting device, which device comprises drug release means including an anti-PESC agent, which device when deployed in a patient positions the drug release means proximal to target epithelial tissue and releases the agent in an amount sufficient to achieve a therapeutically effective exposure of the target epithelial tissue to the agent.

In certain embodiments, the target epithelial tissue is esophageal tissue.

In certain embodiments, the target epithelial tissue is an epithelial-derived tumor, such as an ovarian tumor, a lung tumour, a gastric tumor or an esophageal tumor, or a metastatic site thereof.

For instance, the drug eluting device can produce at least a minimally effective concentration (MEC) of the anti-PESC agent in the target epithelial tissue to which it is applied to which it is applied for at least 30 minutes, more preferably at least 60, 120, 180, 240 or even 300 minutes.

For instance, the drug eluting device can produce anti-PESC agent concentration in the esophageal tissue to which it is applied with T1/2 of at least 2 hours, more preferably at least 4, 6, 8, 10 or even 12 hours.

In certain embodiments, the drug eluting device produces a systemic concentration of the anti-PESC agent which is less than ⅓^{rd} the maximum tolerated does (MTD) for that agent, and even more preferably less than ⅕^{th}, 1/10^{th} , 1/20^{th}, 1/50^{th} or even 1/100^{th} the maximum tolerated does (MTD) for that agent.

In certain embodiments, the drug eluting device is for treating one or more of esophagitis, Barrett's esophagus, esophageal dysplasia or esophageal cancer, which device comprises drug release means including an Anti-BESC Agent that selectively kills or inhibits the proliferation or differentiation of Barrett's Esophagus stem cells (BESC) relative to normal esophageal stem cells, which device when deployed in a patient positions the drug release means proximal to the luminal surface of the esophagus and releases the agent in an amount sufficient to achieve a therapeutically effective exposure of the luminal surface to the agent.

Exemplary drug eluting devices include biodegradable stents, self-expandable stents, such as a self-expandable metallic stent (SEMS) or self-expandable plastic stent (SEPS), chips and wafers for submucusal implantation, and the like.

In other embodiments, the drug eluting device is a device for extraluminal placement, such as a microneedle cuff.

In certain embodiments, the anti-PESC agent is co-administered with an analgesic, and an anti-infective or both. These may be administered as separate formulation, or optionally, may be the anti-PESC agent is co-formulated with the analgesic or the anti-infective or both.

In certain embodiments, the anti-PESC agent is formulated as a liquid for oral delivery to the esophagus.

In certain embodiments, the anti-PESC agent is formulated as a single oral dose.

In certain embodiments, the anti-PESC agent is delivered by a drug eluting device that is a drug eluting stent.

In certain embodiments, the anti-PESC agent is delivered by a drug eluting device that is a balloon catheter having a surface coating including the agent.

In certain embodiments, the anti-PESC agent is cell permeable, such as characterized by a permeability coefficient of 10⁻⁹ or greater, more preferably 10⁻⁸ or greater or 10⁻⁷ or greater.

In certain embodiments, the anti-PESC agent is a histone demethylase inhibitor. In certain preferred embodiments, the anti-PESC agent is a JmjC inhibitor. For instance, it can be a JmjC inhibitor that binds to and inhibits a catalytic JmjC domain. In other instances, it can be a JmjC inhibitor that is a plant homodomain (PHD) inhibitor or a protein-protein interaction inhibitor.

In certain embodiments, the anti-PESC agent is a pan-JmjC demethylase inhibitor. An exemplary JmjC inhibitor is JIB04. Other exemplary JmjC demethylase inhibitors, including pan-JmjC demethylase inhibitors, are described herein.

In certain embodiments, the anti-PESC agent is a receptor tyrosine kinase inhibitor.

In certain preferred embodiments, the receptor tyrosine kinase inhibitor is an EGFR inhibitor, a HER2 inhibitor or a dual EGFR/HER2 inhibitor.

In certain embodiments, the anti-PESC agent is is a proteasome inhibitor.

In certain embodiments, the anti-PESC agent is a STAT inhibitor, and preferably can be a STAT3 inhibitor.

In certain embodiments, the anti-PESC agent is is a FLT3 inhibitor.

In certain embodiments, the anti-PESC agent is is a GSK3 inhibitor.

In certain embodiments, the anti-PESC agent is is a HSP90 inhibitor, a HSP70 inhibitor or a dual HSP90/HSP70 inhibitor.

In certain embodiments, the anti-PESC agent is a selected from the group consisting of:

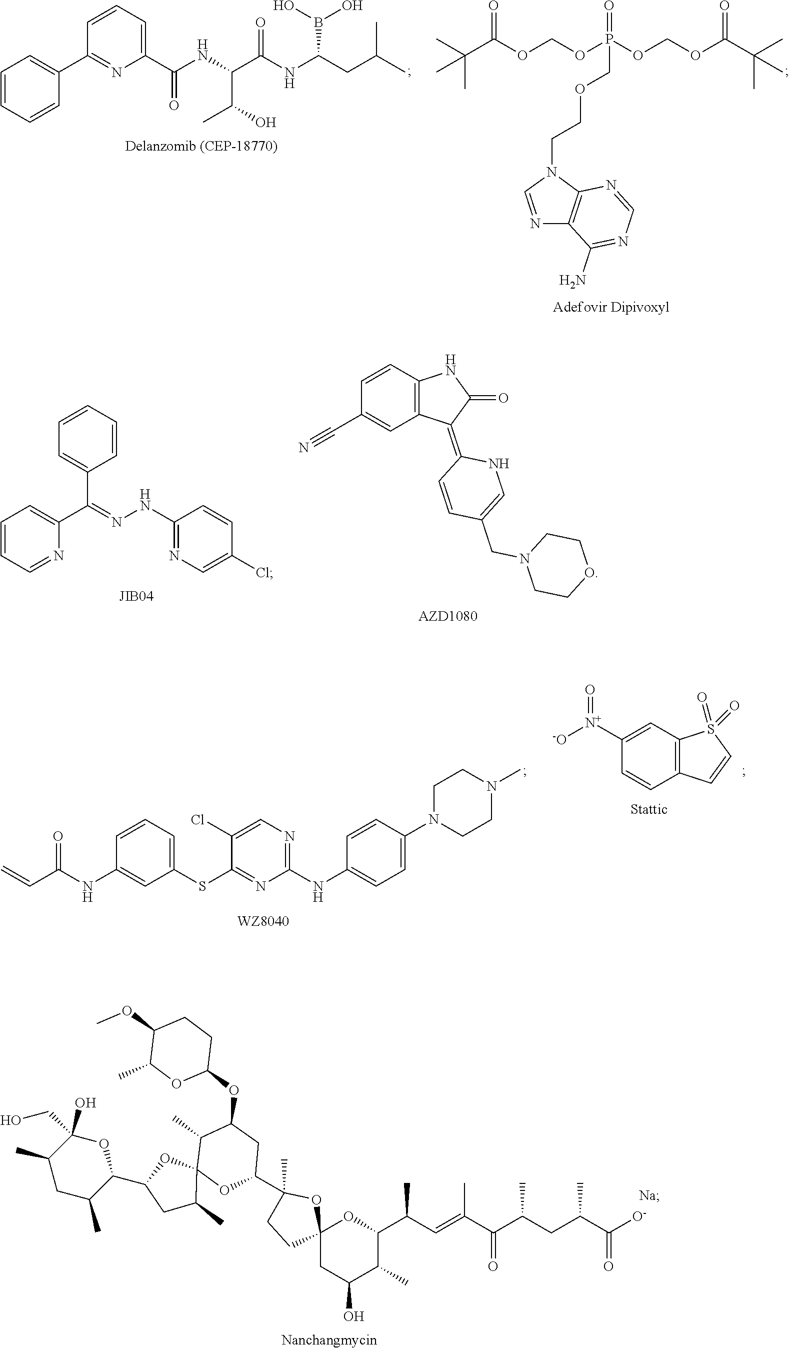

and

In certain embodiments of the methods, preparations and devices of the present disclosure the anti-PESC agent is administered with a second drug agent that selectively promotes proliferation or other regenerative and wound healing activities of normal epithelial stem cells (an “ESO Regenerative agent”) with an EC₅₀ at least 5 times more potent than for PESCs, more preferably with an EC₅₀ 10 times, 50 times, 100 times or even 1000 times more potent for normal epithelial stem cells relative to for PESCs.

In certain embodiments of the methods, preparations and devices of the present disclosure the anti-PESC agent is administered with a second drug agent that selectively promotes proliferation or other regenerative and wound healing activities of normal esophageal stem cells (an “esophageal ESO Regenerative agent”) with an EC₅₀ at least 5 times more potent than for BESCs, more preferably with an EC₅₀ 10 times, 50 times, 100 times or even 1000 times more potent for normal esophageal stem cells relative to for BESCs.

In certain embodiments of the methods, preparations and devices of the present disclosure the anti-PESC agent is administered with an ESO Regenerative agent that selectively promotes proliferation of normal epithelial stem cells with an EC₅₀ of 10⁻⁶ M or less, more preferably 10⁻⁷ M or less, 10⁻⁸ M or less or 10⁻⁹ M or less.

In certain embodiments of the methods, preparations and devices of the present disclosure the anti-PESC agent is administered with an esophageal ESO Regenerative agent that selectively promotes proliferation of normal esophageal stem cells with an EC₅₀ of 10⁻⁶ M or less, more preferably 10⁻⁷ M or less, 10⁻⁸ M or less or 10⁻⁹ M or less.

In certain embodiments, the combined administration of the anti-PESC agent and the ESO Regenerative agent has a therapeutic index (TI) for treating esophagitis, Barrett's Esophagus, esophageal dysplasia and/or esophageal cancer of at least 2, and more preferably has a therapeutic index of at least 5, 10. 20. 50. 100, 250. 500 or 1000 for treating esophagitis, Barrett's Esophagus, esophageal dysplasia and/or esophageal cancer.

In certain embodiments, the combined administration of the anti-PESC agent and the ESO Regenerative agent has a therapeutic index (TI) for treating ovarian, fallopian and or cervical metaplasia or dysplasia of at least 2, and more preferably has a therapeutic index of at least 5, 10, 20, 50, 100, 250, 500 or 1000.

In certain embodiments, the combined administration of the anti-PESC agent and the ESO Regenerative agent has a therapeutic index (TI) for treating ovarian cancer (such as taxol and/or cisplatin resistant ovarian cancer) of at least 2, and more preferably has a therapeutic index of at least 5, 10, 20, 50, 100, 250, 500 or 1000.

In certain embodiments, the combined administration of the anti-PESC agent and the ESO Regenerative agent has a therapeutic index (TI) for treating lung cancer (such NSCLC or SCLC) of at least 2, and more preferably has a therapeutic index of at least 5, 10, 20, 50, 100, 250, 500 or 1000.

In certain embodiments, the combined administration of the anti-PESC agent and the ESO Regenerative agent has a therapeutic index (TI) for treating lung metaplasia or dysplasia of at least 2, and more preferably has a therapeutic index of at least 5, 10, 20, 50, 100, 250, 500 or 1000.

In certain embodiments, the ESO Regenerative agent is pan-inhibitor of ABL kinase inhibitor, preferably a BCR-ABL kinase inhibitor. Exemplary pan-inhibitor include imatinib, nilotinib, dasatinib, bosutinib and ponatinib, and is preferably ponatinib.

In certain embodiments, the ESO Regenerative agent is a tyrosine kinase inhibitor, which at its Cmax concentration following its route of administration, inhibits one or more of FLT3, Bcr-Abl, c-KIT, PDGFR, VEGFR and FGFR with a Ki for inhibition that is less than ½ the EC₅₀ for slowing or reversing the progress of an esophageal metaplasia, dysplasia, cancer or a combination thereof, and even more preferably ⅕^{th}, 1/10^{th}. 1/20^{th}, 1/50^{th} or even 1/100^{th}.

In certain embodiments, the anti-PESC agent and the ESO Regenerative agent are administered to the patient as separate formulations.

In certain embodiments, the anti-PESC agent and the ESO Regenerative agent are co-formulated together.

One aspect of the disclosure provides a single oral dosage formulation comprising (i) an anti-PESC agent, (ii) an ESO Regenerative Agent, and (iii) and a pharmaceutically acceptable excipient, which single oral dosage formulation taken by an adult human patient produces a concentration of anti_PESC agent and ESO Regenerative Agent in esophageal tissue effective to slow or reverse the progress of an esophageal metaplasia, dysplasia, cancer or a combination thereof. In certain preferred embodiments, the BCR-ABL kinase inhibitor is ponatinib. In certain preferred embodiments, the BCR-ABL kinase inhibitor is a FLT3 inhibitor such as quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

In certain embodiments, the methods, preparations and devices of the present disclosure are intended (and appropriate) for use in human patients.

Still another aspect of the present disclosure provides a method for treating a patient suffering from one or more of esophagitis, Barrett's Esophagus, esophageal dysplasia or esophageal cancer, which method comprises administering to the esophagus of a patient a nucleic acid construct that reduces the level of expression of one or more of JmjC, EGFR, HER2, proteasome subunits, STAT3, FLT3, GSK3, HSP90 and/or HSP70 to selectively kill or inhibit the proliferation or differentiation of Barrett's Esophagus stem cells (BESC) relative to normal esophageal stem cells. Exemplary nucleic acid constructs include RNAi constructs (nucleic acids which reduce expression through an RNA interference mechanism) such as siRNA, shRNA or miRNA, as well as antisense nucleic acids. In certain embodiments, the patient presents with esophagitis. In certain embodiments, the patient presents with Barrett's Esophagus. In certain embodiments, the patient presents with esophageal dysplasia. In certain embodiments, the patient presents with esophageal cancer. In certain embodiments, the patient presents with esophageal carcinoma, such as esophageal adenocarcinoma or esophageal squamous cell carcinoma.

Similarly, another aspect of the present disclosure provides a method for treating a patient suffering from one or more of esophagitis, Barrett's Esophagus, esophageal dysplasia or esophageal cancer, which method comprises administering to the esophagus of a patient a CRISPR construct that reduces the level of expression of one or more of JmjC, EGFR, HER2, proteasome subunits, STAT3, FLT3, GSK3, HSP90 and/or HSP70 to selectively kill or inhibit the proliferation or differentiation of BESCs relative to normal esophageal stem cells. In certain embodiments, the patient presents with Barrett's Esophagus. In certain embodiments, the patient presents with esophageal dysplasia. In certain embodiments, the patient presents with esophageal cancer. In certain embodiments, the patient presents with esophageal carcinoma, such as esophageal adenocarcinoma or esophageal squamous cell carcinoma.

### DESCRIPTION OF THE FIGURES

FIGS. 1A-G. Establishment of HTS screening on BE stem cells.

FIG. 1A. Schematic of chemical screening pipeline. 1 mm biopsy of BE lesion was processed and mixed BE stem cells (Krt7+) and ESO stem cell (Krt5+) were cloned and culture in StemECHO cell culture system. Single cell derived pedigrees of Krt5+ ESO stem cells and Krt7+ BE stem cells were established, labeled with GFP and subject to chemical screening. Drugs that eradicate the BE stem cells were identified as hits.

FIG. 1B. Summary of the chemical libraries being used for the screenings.

FIG. 1C. Sensitivity clustering of cell lines used in the screening. BE stem cells and ESO stem cells showed overall distinct sensitivity to chemicals included in the screenings.

FIG. 1D. Left, Scatter plot comparing median survival rate of BE stem cells and ESO stem cells. Right, Ponatinib promoted the proliferation of ESO stem cells.

FIG. 1E. Left, Representative images of ESO and BE stem cells in the absence or presence of Ponatinib. Right, Dosage response of BE and ESO stem cells towards Ponatinib.

FIG. 1F. Representative image of a scanned 384 plate showing the effects of the chemicals on GFP labeled stem cells.

FIG. 1G. Example of converting green binary overlay to the heatmap representing the total area of colonies in each individual well.

FIGS. 2A-F. Synergistic HTS screening to identify dual-function drug combinations.

FIG. 2A. Scatter plot display of the HTS result of synergistic HTS. Selected eight top hits were highlighted in red.

FIG. 2B. Summary of the information of selected top hits.

FIG. 2C. Dosage response curves of the top eights hits validated their differential effects to BE and ESO stem cells.

FIG. 2D. Left, Scanned images of BE (Krt7+, Green)/ESO (Krt5+, Red) co-culture system for the validation of seven selected drugs on eight patients. Right, Quantitative analysis of the scanned images.

FIG. 2E. Scanned image of GFP labeled BE stem cells treated with a gradient of selected hits from HTS.

FIG. 2F. Dosage curves of the hits showed that all selected ones were validated to eradicate BE stem cells. Numeric values obtained from GFP signal threshold image analysis were used to generate dose response curves.

FIGS. 3A-E. Validation of selected hits in 3D and mouse models.

FIG. 3A. Upper, Schematic of the 3D culture validation. Lower, Representative images of top view and histological sections of untreated and treated 3D co-culture structures. BE stem cells (Krt7+, Green) and ESO stem cells (Krt5+, Red) co-existed in the absence of selected drug combination. Following the treatment, BE stem cells (Green) were eradicated while ESO stem cells (Red) compensated the blanked area. FIG. 3B. Quantitative analysis of 3D co-culture treatment images of top view from eight patients.

FIG. 3C. Schematic of drug combination validation in a mouse model. Mice were co-injected subcutaneously with a mixture of BE (GFP labeled) and ESO stem cells. Following the serial treatments, the GFP+ BE stem cells became invisible.

FIG. 3D. Histological analysis of BE stem cells (Krt7+, Green)/ESO stem cells (Krt5+, Red) co-transplanted structures from treated and non-treated mice.

FIG. 3E. Quantitative analysis of clonogenic assay performed by processing the untreated and treated transplants and culturing in StemECHO culture system.

FIG. 3F. Scanned images of BE stem cells and ESO stem cells treated with a range of dosages of eight selected hits together with 1 μM Ponatinib. Their differential effects on BE and ESO stem cells were validated except AZD1080 that appeared to be the false positive hit in the screening.

FIGS. 4A-E. Drug combinations eradicated patient-matched BE, dysplasia and cancer.

FIG. 4A. PCA map of stem cell gene expression from Barrett's, dysplastic, tumor and normal esophagus.

FIG. 4B. Venn diagrams of overlapped genes among BE, dysplasia and cancer stem cells versus ESO stem cells.

FIG. 4C. Common pathways enriched in BE, dysplasia and cancer.

FIG. 4D. Pathways targeted by the hits identified in HTS.

FIG. 4E. Upper, Scanning images of co-cultured BE stem cells (Krt7+, Green) and ESO stem cells (Krt5+, Red) treated with seven different drug combinations. Lower, Quantitative analysis of the scanned images showed that patient-matched BE, dysplasia and cancer stem cells could be eradicated with the same drug combinations.

FIGS. 5A-G. CEP-18770 and JIB04 together with Ponatinib eradicated BE, dysplasia and cancer stem cells in vitro and in vivo.

FIG. 5A. Schematic of 3D culture validation on patient-matched BE, dysplasia and cancer stem cells.

FIG. 5B. Left, Scanned images of top view of BE, dysplasia and cancer stem cells (green) generated ALI structures in the presence of absence of drug combinations. Right, Quantitative analysis of the images of ALI structures showed that CEP-18770 and JIB04 combined with Ponatinib eradicated BE, dysplasia and cancer stem cells.

FIG. 5C. Schematic of in vivo validation of drug combinations in mice xenografted with dysplasia and cancer stem cells. Following the treatment, the xenografts were processed for histological analysis or clonogenic assays to detect the existence of stem cells.

FIG. 5D. Dysplastic structures (Krt7+, Green/Ki67+, Red) were diminished following CEP-19770/Ponatinib or JIB04/Ponatinib treatment. Consistently, stem cells were not cloned from treated structures as shown in scanned rhodamine staining.

FIG. 5E. Cancer structures (Krt7+, Ki67+) were diminished following the drug treatment. Rhodamine staining showed the cancer stem cells were not cloned from the treated structures.

FIGS. 5F and 5G. BE stem cells (Krt7+, Green) and ESO stem cells (Krt5+, Red) were co-injected into the NSG mice and treated with JIB04 or CEP-18770 with Ponatinib. The tissues were collected and fixed for histological analysis. BE stem cells were not detected in the treated structures while the Krt5+ ESO structures were visibly more robust in the treated samples.

FIG. 6. Patient-matched BE, dysplasia and cancer stem cells (Krt7+, Green) were mixed with Krt5+ (red) ESO cells and co-cultured for five days and then treated with seven selected drugs together with 1 μM Ponatinib. Scanning image and quantitative analysis showed that all seven drug combinations reduced the growth of BE, dysplasia and cancer stem cell growth, while JIB04 and CEP-18770 remained the most effective drugs.

FIGS. 7A-7D. Exemplary classes and illustrative structures of histone deacetylase inhibitors useful in the present disclosure.

FIG. 8. Is a diagram representing the continuum in certain epithelial tissues of metaplasia to dysplasia to cancer.

FIG. 9. Is a diagram showing the statistically increasing risk of a patient developing esophageal adenocarcinoma as disease progresses from Barrett's esophagus to high grade dysplasia.

FIG. 10A. Demonstrates the synergy in the combination of JIB04 with ponatinib in killing pathogenic BE stem cells relative to normal esophageal stem cells. Projected therapeutic index is greater than 200 fold.

FIG. 10B. Demonstrates the synergy in the combination of JIB04 with ponatinib in killing both BE stem cells, as well as stem cells from biopsies graded as dysplasia and Esophageal Adenocarcinoma (EAC). Projected therapeutic index is greater than 100 fold.

FIG. 11A. Pathogenic stem cells isolated from high grade ovarian tumor biopsies obtained from drug naïve patients already include cisplatin-resistant stem cell populations. PCA analysis of cisplatin-resistant stem cell populations from drug naïve patients indicates that these cells cluster with pathogenic stem cells from stem cells isolated from cisplatin-resistant tumors from patients having relapsed or failed cisplatin therapy.

FIG. 11B. Demonstrates the synergy in the combination of taxol with ponatinib in killing taxol-resistant ovarian stem cells in vitro.

FIG. 11C. Demonstrates the synergy in the combination of taxol with ponatinib in reducing tumor volume taxol-resistant ovarian stem cells in xenograft animal models.

FIG. 12. Pathogenic stem cells can be cloned and cultured from lung cancers (both small cell and non-small cell lung cancer). These pathogenic stem cells, along with normal regenerative lung stem cells, can be adapted to high throughput screening plates.

FIG. 13. Demonstrates the synergy in the combination of JIB04 with ponatinib in killing both NSCLC and SCLC stem cells relative to normal lung epithelial stem cells.

### DESCRIPTION OF CERTAIN EMBODIMENTS

### I. OVERVIEW

Barrett's Esophagus holds a pivotal position at the interface of cancer biology and patient care. Barrett's was first discovered in 1950's and associated with risk for adenocarcinoma in the 1970's. Barrett's has become a paradigm for precancerous lesions giving rise to progressively more advanced lesions in a process requiring many years supporting an overall escalation model whereby non-cancerous lesions undergo long-term processes of stochastic changes some of which yield more sinister and determinant transitions to low- and high-grade dysplasia which then rapidly and almost inexorably evolve to malignant disease. The recognition of the importance of preemptive therapies that target these premalignant lesions is the foundation of cancer prevention. If true, the clinical solution to preventing the onset of esophageal adenocarcinoma would be simple and direct: ablate Barrett's before it can evolve to more aggressive lesions.

The advance of the development of targeted therapies for Barrett's requires conceptual advance of the origin of Barrett's and the recognition of the existence of Barrett's stem cells. If the premalignant stages of EAC represent the only tractable solution to this disease, it is essential to solve the mystery of the origin of BE and develop new therapeutic strategies specifically targeting its stem cells. However, the ontogeny of BE has been an intriguing puzzle with various hypotheses involving transcommitment of esophageal squamous stem cells, migration from lower gastrointestinal sites, the reparative emergence of submucosal glands, dissemination from bone marrow. We recently showed that BE originated from the opportunistic growth of residual embryonic cells pre-existing at gastroesophageal junction (Wang et al, Cell. 2011 Jun. 24; 145(7):1023-1035). In addition, using the ground state stem cell technology that enabled us to clone stem cells of the normal human gastrointestinal tract, we demonstrated the existence of the stem cells in BE (Yamamoto et al., Nat Commun. 2016 Jan. 19; 7:10380) and suggested they are the key elements to target in a therapeutic program designed to prevent the development and progression of this irreversible and dangerous metaplasia.

In order to uncover drugs specifically targeting BE stem cells that might synergize with physical ablation protocols to further reduce recurrent disease, provided herein is a multiplexed screening of established and experimental drugs or combinations thereof to identify compounds and combinations of compounds that selectively target the particular pathways that dominate the survival of these BE lesions. These BE stem cells were used in hybrid models with normal epithelial squamous stem cells to model the potential ability of such drug combinations to alter the competitive status of such lesions in the distal esophagus.

Also provided herein are screening methods that show the similar selective vulnerabilities of the stem cells of patient-matched BE, dysplasia and EAC, which suggest the broad usage of the pharmacological compositions that would augment physical ablation or mucosal dissection therapies. Indeed, as demonstrated by the data presented herein, the differential sensitivity of the pathogenic stem cells to single agents or combination therapies is carried across multiple tissues and across metaplasia, dysplasia or tumor samples from those tissues.

### II. DEFINITIONS

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like. The term “alkyl,” as used herein, in synonymous with the term “aliphatic.”

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

“Alkylsulfonyl” means a —SO₂R radical where R is alkyl as defined above, e.g., methylsulfonyl, ethylsulfonyl, and the like.

“Alkoxy” means an —OR radical where R is alkyl as defined above, e.g., methoxy, ethoxy, propoxy, or 2-propoxy, n-, iso-, or tert-butoxy, and the like.

“Aminoalkyl” means a linear monovalent hydrocarbon radical of one to six carbon atoms or a branched monovalent hydrocarbon radical of three to six carbons substituted with at least one, preferably one or two, —NRR where R is hydrogen, alkyl, or —COR^{a} where R^{a} is alkyl, each as defined above, and R is selected from hydrogen, alkyl, hydroxyalkyl, alkoxyalkyl, aryl, aralkyl, heteroaryl, heteroaralkyl, or haloalkyl, each as defined herein, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminosulfonyl” means a —SO₂NRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —SO₂NH₂, methylaminosulfonyl, 2-dimethylaminosulfonyl, and the like.

“Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, e.g., acetyl, propionyl, benzoyl, pyridinylcarbonyl, and the like. When R is alkyl, the radical is also referred to herein as alkylcarbonyl.

“Acylamino” means an —NHCOR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, e.g., acetylamino, propionylamino, and the like.

“Aryl” means a monovalent monocyclic or bicyclic aromatic hydrocarbon radical of 6 to 10 ring atoms e.g., phenyl or naphthyl. As used herein, “aryl” and “aromatic” may be used interchangeably.

“Bridged heterocyclyl” means a saturated or unsaturated monovalent bicyclic group of 5 to 10 ring atoms in which one or two ring atoms are heteroatom selected from N, O, or S(0)n where n is an integer from 0 to 2, the remaining ring atoms being C, where some of the rings are created by one or more bridges.

“Cycloalkyl” means a cyclic, saturated, monovalent hydrocarbon radical of three to ten carbon atoms, e.g., cyclopropyl, cyclobutyl, cyclopentyl, or cyclohexyl, and the like. The cycloalkyl ring can optionally be fused to phenyl or monocyclic heteroaryl ring as defined herein. When the cycloalkyl ring is referred to herein as “fused cycloalkyl”, it means that the cycloalkyl ring is fused to phenyl or monocyclic heteroaryl ring. When the cycloalkyl ring is referred to herein as “monocyclic cycloalkyl”, it means that the cycloalkyl ring is not fused to phenyl or monocyclic heteroaryl ring. As used herein, “cycloalkyl,” “carbocycle,” and “carbocylyl” may be used interchangeably.

“Carboxy” means —COOH.

“Disubstituted amino” means an —NRR′ radical where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined herein, e.g., dimethylamino, phenylmethylamino, and the like. When R and R′ are alkyl, the group is referred to herein as dialkylamino

“Halo” means fluoro, chloro, bromo, or iodo, preferably fluoro or chloro. “Haloalkyl” means alkyl radical as defined above, which is substituted with one or more halogen atoms, preferably one to five halogen atoms, preferably fluorine or chlorine, including those substituted with different halogens, e.g., —CH₂C1, —CF₃, —CHF₂, —CH₂CF₃, —CF₂CF₃, —CF(CH₃)₂, and the like. When the alkyl is substituted with only fluoro, it is referred to in this Application as fluoroalkyl.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy ethyl, 2-hydroxypropyl, 3-hydroxypropyl, I-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, or 1-(hydroxymethyl)-2-hydroxyethyl.

“Heterocyclyl” means a saturated or unsaturated monovalent monocyclic group of 4 to 8 ring atoms in which one or two ring atoms are heteroatom selected from N, O, or S(0)n, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this Application as “bicyclic heterocyclyl” ring. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, tetrahydroisoquinolinyl, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

“Heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

“Monosubstituted amino” means an —NHR radical where R is alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined herein, e.g., methylamino, 2-phenylamino, hydroxyethylamino, and the like. When R is alkyl, the group is referred to herein as monoalkylamino.

A “pharmaceutically acceptable salt” of a compound means a salt that is pharmaceutically acceptable and that possesses the desired pharmacological activity of the parent compound. Such salts include: acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-I-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference. The compounds of the present disclosure can also exist as cocrystals.

The compounds of the present disclosure may have asymmetric centers. Compounds of the present disclosure containing an asymmetrically substituted atom may be isolated in optically active, racemic forms or other mixtures of isomers. It is well known in the art how to prepare optically active forms, such as by resolution of materials. All chiral, diastereomeric, racemic forms are within the scope of this disclosure, unless the specific stereochemistry or isomeric form is specifically indicated.

Certain compounds of can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfonyl” means a —SO₂R radical where R is alkyl, haloalkyl, aryl, aralkyl, heteroaryl, heteroaralkyl, heterocyclyl, heterocyclylalkyl, each as defined herein, e.g., methylsulfonyl, phenylsulfonyl, benzylsulfonyl, pyridinylsulfonyl, and the like. When R is alkyl, it is also referred to herein as alkylsulfonyl.

“Substituted alkyl” means a linear saturated monovalent hydrocarbon radical of one to six carbon atoms or a branched saturated monovalent hydrocarbon radical of three to six carbon atoms where one or two hydrogen atoms in the alkyl chain are independently replaced by hydroxyl, halo, alkoxy, amino, monosubstituted amino, disubstituted amino, cyano, sulfonyl, aminocarbonyl, aminosulfonyl, —NHCONH₂, carboxy, acyl, acylamino, phenyl, or alkoxycarbonyl, each group as defined herein.

“Substituted alkynyl” means a linear saturated monovalent hydrocarbon radical of two to six carbon atoms or a branched monovalent hydrocarbon radical of three to six carbon atoms containing a triple bond where one or two hydrogen atoms in the alkynyl chain are independently replaced by phenyl, hydroxyl, alkoxy, amino, monosubstituted amino, disubstituted amino, cyano, sulfonyl, aminocarbonyl, aminosulfonyl, —NHCONH₂, carboxy, acyl, acylamino, or alkoxycarbonyl, each group as defined herein.

“Treating” or “treatment” of a disease includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

### III. EXEMPLARY EMBODIMENTS

a. Histone Demethylase Inhibitors

The Jumonji family of histone demethylases has been the focus of much study over the last few years. Numerous reports have demonstrated the relevance of these enzymes in a variety of physiological and pathological conditions beyond cancer, including early development, reproduction, metabolism, and cardiac hypertrophy. The structure of Jumonji catalytic domains shows they are drugable, able to accommodate small molecule disruptors, making them ideal molecular targets for intervention. Modulation of aberrant Jumonji demethylase activity in disease should lead to the normalization of transcriptional patterns, such as we see with JIB04 in cancer cells. JIB04's ability to block tumor growth and prolong cancer survival may involve both direct and indirect aggregate effects of Jumonji enzyme pan-inhibition in cells and in vivo, and it is possible that the drug accumulate in cancer cells over time increasing its effective concentration/apparent potency. Mechanistically, JIB04 appears to chelate iron in the catalytic site of Jumonji enzymes and to disrupt histone substrate binding, while not being a competitive inhibitor for a-ketoglutarate, a mechanism not yet described for Jumonji inhibitors. Future structural information will be necessary to establish the exact molecular interactions between Jumonji enzymes and their cofactors/substrates that are disrupted by JIB04. One possibility is that the inhibitor may occupy the outer portion of the active site where the iron and the histone substrate bind or bind iron in the active site in a manner that alters subsequent substrate binding. Chelation in solution may also contribute to inhibition under conditions of high free iron.

In certain embodiments, the anti-PESC agent is JIB-004

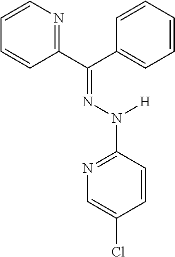 [SMILES: Clc1ccc(N/N=C(\c2ccccc2)c2ccccn2)nc1]

JIB04 (NSC693627, E-isomer) is a potent, selective and cell permeable Jumonji histone demethylase inhibitor. Unlike the other known inhibitors, JIB04 is not a competitive inhibitor of a-ketoglutarate. It inhibits the demethylase activity of Jumonji enzymes in vitro, with IC₅₀˜230 nM for JARID1A (KDM5A), ˜440 nM for JMJD2A (KDM4A) and JMJD2B (KDM4B), ˜340 nM for JMJD2E (KDM2E), and ˜1 μM for JMJD3 (KDM6B) and JMJD2C (KDM4C). JIB04 blocks Jumonji demethylase activity in cells and consequently inhibits cell growth, without affecting other a-ketoglutarate-dependent hydroxylases or histone-modifying enzymes, especially HDACs. JIB04 alters transcriptional programs in cancer but not in normal cells, leading to cancer-specific cell death. Importantly, in vivo, JIB04 lowers histone demethylase activity in tumors, reduces tumor burden and prolongs survival of mice in an aggressive breast cancer model.

In some embodiments, the agent that inhibits a JmjC polypeptide is JIB04, SD-70, ML324, KDM5-C70, PBIT, KDOHP64a, KDOQZ5, IOX1, IOX2, KDOMA83, KDMOBP69, NSC636819, pyrido[3,4-pyrimidin-4(3H)-one derivatives, 3-amino-4-pyridine carboxylate derivatives or analogs thereof. See PCT WO2017190009A1

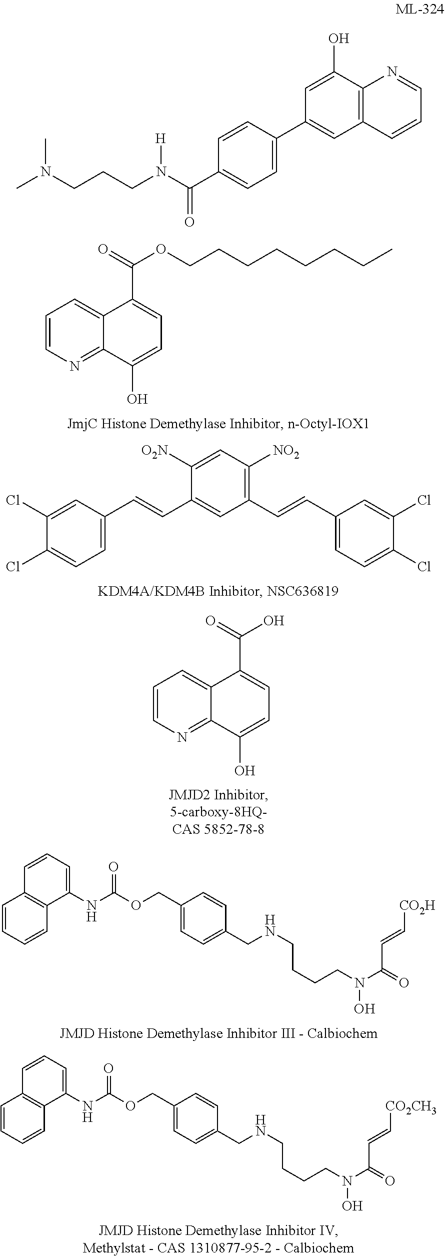

JIB04 as well as other suitable histone demethylase inhibitors are also described in Thinnes et al. Biochimica et Biophysica Acta—Gene Regulatory Mechanisms, Volume 1839, Issue 12, December 2014, Pages 1416-1432 and Epigenetic Drug Discovery ed. Wolfgang Sippl, Manfred Jung, Raimund Mannhold, Helmut Buschmann, Jörg Holenz, John Wiley & Sons, Feb. 11, 2019 ISBN: 978-3-527-34314-0—(both of which are incorporated by reference herein).

In one embodiment, the JMJD3 demethylase inhibitor comprises ethyl 3-((6-(4,5-dihydro-1H-benzo[d]azepin-3(2H)-yl)-2-(pyridin-2-yl)pyrimidin-4-yl)amino)propanoate (GSK-J4), and active derivatives thereof as disclosed in Kruidenier et al., “A Selective Jumonji H3K27 Demethylase Inhibitor Modulates Proinflammatory Macrophage Response,” Nature 488:404-408 (2012), which is hereby incorporated by reference in its entirety. GSK-J4 has the structure of:

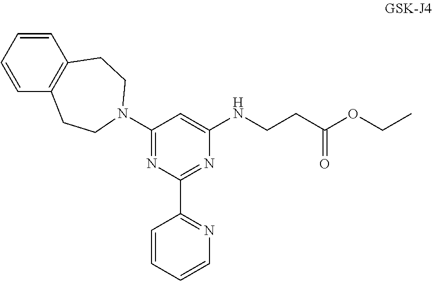 [SMILES: CCOC(=O)CCNc1cc(N2CCc3ccccc3CC2)nc(-c2ccccn2)n1]

Exemplary active derivatives of GSK4 that are also JMJD3 demthylase inhibitors include GSK-J1 and GSK-J3, which have the following structures:

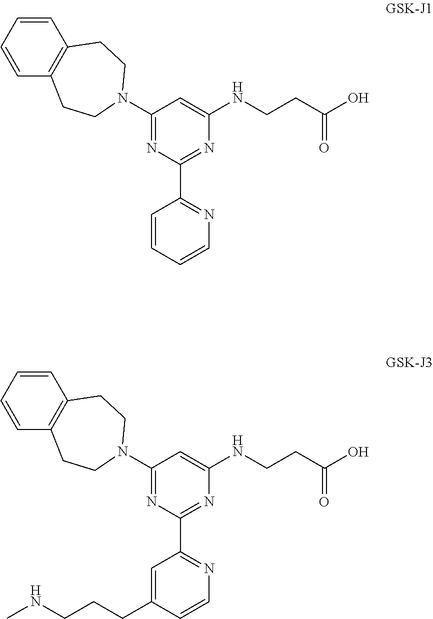 [SMILES: O=C(O)CCNc1cc(N2CCc3ccccc3CC2)nc(-c2ccccn2)n1 ; CNCCCc1ccnc(-c2nc(NCCC(=O)O)cc(N3CCc4ccccc4CC3)n2)c1]

In another embodiment, the JMJD3 demethylase inhibitor comprises a modified GSK-J1 small molecule as described by Hu et al., “Design and Discovery of New Pyrimidine Coupled Nitrogen Aromatic Rings as Chelating Groups of JMJD3 Inhibitors,” Bioorg. Med. Chem. Lett. 26(3):721-725 (2016), which is hereby incorporated by reference in its entirety.

In certain embodiments, the present disclosure provides a compound of formula

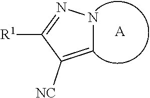

or a pharmaceutically acceptable salt thereof, wherein

R1 is —R, halogen, —OR, —SR, —N(R′)2, —CN, —NO2, —C(O)R, —CO2R, —C(O)N(R′)2, —C(O)SR, —C(O)C(O)R, —C(O)CH2C(O)R, —C(S)N(R′)2, —C(S)OR, —S(O)R, —SO2R, —SO2N(R′)2, —N(R′)C(O)R, —N(R′)C(O)N(R′)2, —N(R′)SO2R, —N(R′)SO2N(R′)2, —N(R′)N(R′)2, —N(R′)C(═N(R′))N(R′)2, —C═NN(R′)2, —C═NOR, —C(═N(R′))N(R′)2, —OC(O)R, or —OC(O)N(R′)2, wherein R and R′ are as defined above and described herein. In some embodiments, R1 is hydrogen. In some embodiments, R1 is optionally substituted C1-6 aliphatic. In certain embodiments, R1 is optionally substituted C1-6 alkyl, C2-6 alkenyl, or C2-6alkynyl. In certain embodiments, R1 is optionally substituted C1-6 alkyl. In certain embodiments, R1 is methyl. In certain other embodiments, R1 is ethyl or tert-butyl. In some embodiments, R1 is —OR, —SR, or —N(R′)2. In certain embodiments, R1 is —SR. In certain embodiments, R1 is —NH2. In certain embodiments, R1 is —CN or —NO2. In some embodiments, R1 is halogen. In certain embodiments, R1 is fluoro, chloro, bromo, or iodo. In certain embodiments, R1 is fluoro. In some embodiments, R1 is —C(O)R, —CO2R, —C(O)SR, —C(O)N(R′)2, —C(O)C(O)R, or —C(O)CH₂C(O)R. In certain embodiments, R1 is —C(S)OR or —C(S)N(R′)2. In other embodiments, R1 is —S(O)R, —SO2R, or —SO2N(R′)2. In some embodiments, R1 is —N(R′)C(O)R, —N(R′)C(O)N(R′)2, —N(R′)SO2R, —N(R′)SO2N(R′)2, —N(R′)N(R′)2, or —N(R′)C(═N(R′))N(R′)2. In certain embodiments, R1 is —N(R′)N(R′)2. In some embodiments, R1 is —C═NN(R′)2, —C═NOR, —C(═N(R′))N(R′)2, —OC(O)R, or —OC(O)N(R′)2;

Ring A is

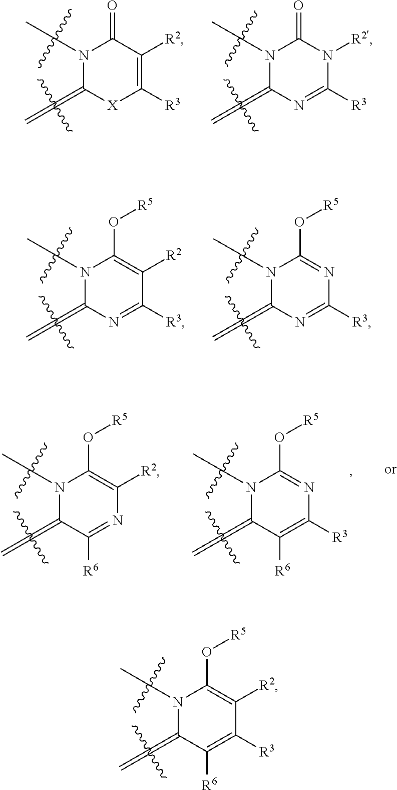

wherein X, R2, R2′, R3, R5, and R6 are as defined above and described herein. Thus, in certain embodiments, a compound of the disclosure is of one of the following formulae:

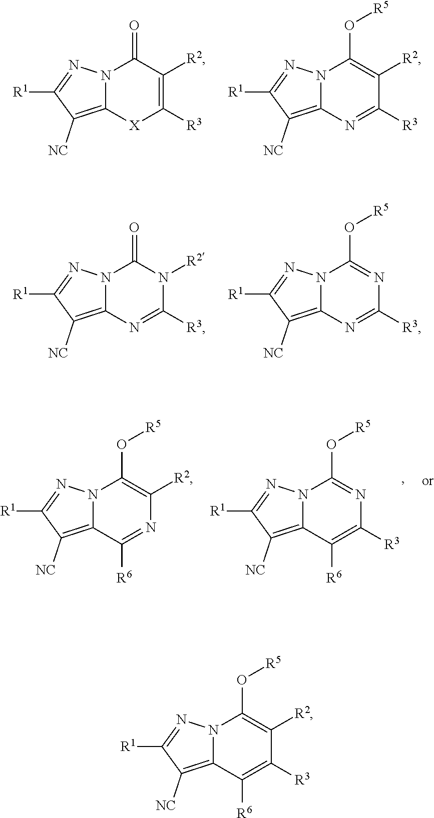

wherein R1, R2, R2′, R3, R5, R6, and X are as defined above and described herein.

As defined generally above, R2 is —R, halogen, —OR, —SR, —N(R′)2, —CN, —NO2, —C(O)R, —CO2R, —C(O)N(R′)2, —C(O)SR, —C(O)C(O)R, —C(O)CH₂C(O)R, —C(S)N(R′)2, —C(S)OR, —S(O)R, —SO2R, —SO2N(R′)2, —N(R′)C(O)R, —N(R′)C(O)N(R′)2, —N(R′)SO2R, —N(R′)SO2N(R′)2, —N(R′)N(R′)2, —N(R′)C(═N(R′))N(R′)2, —C═NN(R′)2, —C═NOR, —C(═N(R′))N(R′)2, —OC(O)R, or —OC(O)N(R′)2, wherein R and R′ are as defined above and described herein. In some embodiments, R2 is hydrogen. In some embodiments, R2 is optionally substituted C1-6 aliphatic. In certain embodiments, R2 is optionally substituted C1-6 alkyl, C2-6 alkenyl, or C2-6 alkynyl. In certain embodiments, R2 is optionally substituted C1-6 alkyl. In certain embodiments, R2 is ethyl. In certain other embodiments, R2 is methyl, propyl, isopropyl, butyl, or isobutyl. In some embodiments, R2 is C1-6 alkyl substituted with an —OH or —OC1-6alkyl group. In certain embodiments, R2 is —CH2CH2OH or —CH2CH2OCH3. In some embodiments, R2 is cycloalkyl. In certain embodiments, R2 is cyclopropyl, cyclobutyl, cyclopentyl, or cyclohexyl. In some embodiments, R2 is optionally substituted C2-6 alkenyl. In certain embodiments, R2 is allyl. In some embodiments, R2 is optionally substituted C2-6 alkynyl. In certain embodiments, R2 is 2-propynyl. In some embodiments, R2 is optionally substituted benzyl. In certain embodiments, R2 is unsubstituted benzyl. In certain other embodiments, R2 is substituted benzyl. In some embodiments, R2 is C1-6 alkyl substituted with an ester group. In certain embodiments, R2 is —CH2CO2C1-6alkyl or —CH2CO2aryl. In certain embodiments, R2 is —CH2CO2CH2CH3. In some embodiments, R2 is —OR, —SR, or —N(R′)2. In certain embodiments, R2 is —CN or —NO2. In some embodiments, R2 is halogen. In certain embodiments, R2 is fluoro, chloro, bromo, or iodo. In some embodiments, R2 is —C(O)R, —CO2R, —C(O)SR, —C(O)N(R′)2, —C(O)C(O)R, or —C(O)CH₂C(O)R. In certain embodiments, R2 is —C(S)OR or —C(S)N(R′)2. In other embodiments, R2 is —S(O)R, —SO2R, or —SO2N(R′)2. In some embodiments, R2 is —N(R′)C(O)R, —N(R′)C(O)N(R′)2, —N(R′)SO2R, —N(R′)SO2N(R′)2, —N(R′)N(R′)2, or —N(R′)C(═N(R′))N(R′)2. In some embodiments, R2 is —C═NN(R′)2, —C═NOR, —C(═N(R′))N(R′)2, —OC(O)R, or —OC(O)N(R′)2.

As defined generally above, R2′ is —R, —OR, —SR, —N(R′)2, —C(O)R, —CO2R, —C(O)N(R′)2, —C(O)SR, —C(O)C(O)R, —C(O)CH₂C(O)R, —C(S)N(R′)2, —C(S)OR, —S(O)R, —SO2R, —SO2N(R′)2, —N(R′)C(O)R, —N(R′)C(O)N(R′)2, —N(R′)SO2R, —N(R′)SO2N(R′)2, —N(R′)N(R′)2, —N(R′)C(═N(R′))N(R′)2, —C═NN(R′)2, —C═NOR, —C(═N(R′))N(R′)2, —OC(O)R, or —OC(O)N(R′)2, wherein R and R′ are as defined above and described herein. In some embodiments, R2′ is hydrogen. In some embodiments, R2′ is optionally substituted C1-6 aliphatic. In certain embodiments, R2′ is optionally substituted C1-6 alkyl, C2-6 alkenyl, or C2-6 alkynyl. In certain embodiments, R2′ is optionally substituted C1-6 alkyl. In certain embodiments, R2′ is ethyl. In certain other embodiments, R2′ is methyl, propyl, isopropyl, butyl, or isobutyl. In some embodiments, R2′ is C1-6 alkyl substituted with an —OH or —OC1-6alkyl group. In certain embodiments, R2′ is —CH2CH2OH or —CH2CH2OCH3. In some embodiments, R2′ is cycloalkyl. In certain embodiments, R2′ is cyclopropyl, cyclobutyl, cyclopentyl, or cyclohexyl. In some embodiments, R2′ is optionally substituted C1-6 alkenyl. In certain embodiments, R2′ is allyl. In some embodiments, R2′is optionally substituted C1-6 alkynyl. In certain embodiments, R2′ is 2-propynyl. In some embodiments, R2′ is optionally substituted benzyl. In certain embodiments, R2′ is unsubstituted benzyl. In certain other embodiments, R2′ is substituted benzyl. In some embodiments, R2′ is C1-6 alkyl substituted with an ester group. In certain embodiments, R2′is —CH2CO2C1-6alkyl or —CH2CO2aryl. In certain embodiments, R2′ is —CH2CO2CH2CH3. In some embodiments, R2′ is —OR, —SR, or —N(R′)2. In some embodiments, R2′ is —C(O)R, —CO2R, —C(O)SR, —C(O)N(R′)2, —C(O)C(O)R, or —C(O)CH2C(O)R. In certain embodiments, R2′ is —C(S)OR or —C(S)N(R′)2. In other embodiments, R2′ is —S(O)R, —SO2R, or —SO2N(R′)2. In some embodiments, R2′ is —N(R′)C(O)R, —N(R′)C(O)N(R′)2, —N(R′)SO2R, —N(R′)SO2N(R′)2, —N(R′)N(R′)2, or —N(R′)C(═N(R′))N(R′)2. In some embodiments, R2′ is —C═NN(R′)2, —C═NOR, —C(═N(R′))N(R′)2, —OC(O)R, or —OC(O)N(R′)2.

As defined generally above, R3 is —R, halogen, —OR, —SR, —N(R′)2, —CN, —NO2, —C(O)R, —CO2R, —C(O)N(R′)2, —C(O)C(O)R, —C(O)CH2C(O)R, —S(O)R, —SO2R, —SO2N(R′)2, —N(R′)C(O)R, —N(R′)C(O)N(R′)2, —N(R′)SO2R, —N(R′)SO2N(R′)2, —N(R′)N(R′)2, —C═NN(R′)2, —C═NOR, —OC(O)R, or —OC(O)N(R′)2, wherein R and R′ are as defined above and described herein. In some embodiments, R3 is hydrogen. In some embodiments, R3 is optionally substituted C1-6aliphatic. In certain embodiments, R3 is optionally substituted C1-6 alkyl, C2-6 alkenyl, or C2-6 alkynyl. In certain embodiments, R3 is optionally substituted C1-6 alkyl. In certain embodiments, R3 is methyl. In certain other embodiments, R3 is ethyl, propyl, isopropyl, butyl, or isobutyl. In certain embodiments, R3 is —CF3. In some embodiments, R3 is C1-6 alkyl substituted with an —OH or —OC1-6alkyl group. In certain embodiments, R3 is —CH2OH, —CH2CH2OH, —CH2CH2CH2OH, —CH2OCH2CH3, —CH2OCH3, —CH2CH2CH2OCH3, —CH(OH)CH3, or —CH2CH2OCH3. In some embodiments, R3 is C1-6 alkyl substituted with an —NHC1-6alkyl or —N(C1-6alkyl)2 group. In certain embodiments, R3 is —CH2NHC1-6alkyl. In certain embodiments, R3 is —CH2NHCH3. In some embodiments, R3 is C1-6 alkyl substituted with an aryl, heteroaryl, carbocyclyl, or heterocyclyl ring. In some embodiments, R3 is optionally substituted benzyl. In certain embodiments, R3 is unsubstituted benzyl. In certain other embodiments, R3 is substituted benzyl. In certain embodiments, R3 is —C(R∘)2Ph. In certain embodiments, R3 is —C(R∘)2Ph, wherein R∘ is hydrogen or methyl. In certain embodiments, R3 is trifluoromethylbenzyl. In certain embodiments, R3 is —C(R∘)2(heteroaryl). In certain embodiments, R3 is —C(R∘)2(heteroaryl), wherein the heteroaryl is pyridyl, pyrimidyl, pyrazinyl, pyridazinyl, triazinyl, pyridinonyl, pyrrolyl, pyrazolyl, imidazolyl, triazolyl, tetrazolyl, thienyl, furanyl, thiazolyl, isothiazolyl, thiadiazolyl, oxazolyl, isoxazolyl, or oxadiazolyl. In certain embodiments, R3 is —CH2(heteroaryl), wherein the heteroaryl is pyridyl, pyrimidyl, pyrazinyl, pyridazinyl, triazinyl, pyrrolyl, pyrazolyl, imidazolyl, triazolyl, tetrazolyl, thienyl, furanyl, thiazolyl, isothiazolyl, thiadiazolyl, oxazolyl, isoxazolyl, or oxadiazolyl. In certain embodiments, R3 is —C(R∘)2(carbocyclyl). In certain embodiments, R3 is —C(R∘)2(carbocyclyl), wherein the carbocyclyl is cyclopropyl, cyclobutyl, cyclopentyl, cyclohexyl, or cycloheptyl. In certain embodiments, R3 is —CH2(carbocyclyl), wherein the carbocyclyl is cyclopropyl, cyclobutyl, cyclopentyl, cyclohexyl, or cycloheptyl. In certain embodiments, R3 is —C(R∘)2(heterocyclyl). In certain embodiments, R3 is —C(R∘)2(heterocyclyl), wherein the heterocyclyl is tetrahydrofuranyl, tetrahydrothienyl, tetrahydropyranyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. In certain embodiments, R3 is —CH2(heterocyclyl), wherein the heterocyclyl is tetrahydrofuranyl, tetrahydrothienyl, tetrahydropyranyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. In some embodiments, R3 is optionally substituted C2-6 alkenyl. In certain embodiments, R3 is allyl. In some embodiments, R3 is optionally substituted C2-6 alkynyl. In certain embodiments, R3 is propargyl. In some embodiments, R3 is an optionally substituted aryl or heteroaryl group. In certain embodiments, R3 is phenyl. In certain embodiments, R3 is substituted phenyl. In certain embodiments, R3 is toluyl. In certain other embodiments, R3is a 5-6 membered heteroaryl ring having 1-3 heteroatoms selected from nitrogen, oxygen, and sulfur. In certain embodiments, R3 is pyridyl, pyrimidyl, pyrazinyl, pyridazinyl, triazinyl, pyrrolyl, pyrazolyl, imidazolyl, triazolyl, tetrazolyl, thienyl, furanyl, thiazolyl, isothiazolyl, thiadiazolyl, oxazolyl, isoxazolyl, or oxadiazolyl. In some embodiments, R3 is —OR, —SR, or —N(R′)2. In some embodiments, R3 is halogen. In certain embodiments, R3 is fluoro, chloro, bromo, or iodo. In some embodiments, R3 is —C(O)R, —CO2R, —C(O)N(R′)2, —C(O)SR, —C(O)C(O)R, or —C(O)CH₂C(O)R. In certain embodiments, R3 is optionally substituted —CO2C1-6alkyl. In certain embodiments, R3 is —CO2Et or —CO2Bn. In certain embodiments, R3 is —CONHC1-6alkyl. In certain embodiments, R3 is —CONHCH3 or —CONHCH2CH3. In certain embodiments, R3 is —C(S)OR or —C(S)N(R′)2. In other embodiments, R3 is —S(O)R, —SO2R, or —SO2N(R′)2. In some embodiments, R3 is —N(R′)C(O)R, —N(R′)C(O)N(R′)2, —N(R′)SO2R, —N(R′)SO2N(R′)2, —N(R′)N(R′)2, or —N(R′)C(═N(R′))N(R′)2. In some embodiments, R3 is —C═NN(R′)2, —C═NOR, —C(═N(R′))N(R′)2, —OC(O)R, or —OC(O)N(R′)2.

In some embodiments, R2 and R3 are taken together with their intervening atoms to form an optionally substituted 5-7 membered partially unsaturated or aromatic fused ring having 0-4 heteroatoms independently selected from nitrogen, oxygen, and sulfur. In certain embodiments, R2 and R3 are taken together with their intervening atoms to form a 5-membered fused ring. In certain embodiments, R2 and R3 are taken together with their intervening atoms to form a fused cyclopentene ring. In certain embodiments, R2 and R3 are taken together with their intervening atoms to form a 6-membered fused ring. In certain embodiments, R2 and R3 are taken together with their intervening atoms to form a fused cyclohexene ring. In certain embodiments, R2 and R3 are taken together with their intervening atoms to form a fused benzene ring. In certain embodiments, R2 and R3 are taken together with their intervening atoms to form a 5-7 membered partially unsaturated fused ring having 1-4 heteroatoms independently selected from nitrogen, oxygen, and sulfur. In certain embodiments, R2 and R3 are taken together with their intervening atoms to form a 5-7 membered aromatic fused ring having 1-4 heteroatoms independently selected from nitrogen, oxygen, and sulfur.

In some embodiments, R2′ and R3 are taken together with their intervening atoms to form an optionally substituted 5-7 membered partially unsaturated or aromatic fused ring having 1-4 heteroatoms independently selected from nitrogen, oxygen, and sulfur. In certain embodiments, R2′ and R3 are taken together with their intervening atoms to form a 5-membered fused ring. In certain embodiments, R2′ and R3are taken together with their intervening atoms to form a 6-membered fused ring. In certain embodiments, R2′ and R3 are taken together with their intervening atoms to form a fused pyridine ring. In certain embodiments, R2′ and R3 are taken together with their intervening atoms to form a 5-7 membered partially unsaturated fused ring having 1-4 heteroatoms independently selected from nitrogen, oxygen, and sulfur. In certain embodiments, R2′ and R3 are taken together with their intervening atoms to form a 5-7 membered aromatic fused ring having 1-4 heteroatoms independently selected from nitrogen, oxygen, and sulfur.

As defined generally above, X is —N(R4)-, —O—, or —S—, wherein R4 is as defined above and described herein. In certain embodiments, X is —O— or —S—. In some embodiments, X is —N(R4)-. In certain embodiments, X is —NH—. In certain embodiments, X is —N(CH3)-.

As defined generally above, R4 is —R, —C(O)R, —CO2R, or —S(O)2R, or R4 and R3 are taken together with their intervening atoms to form an optionally substituted 5-7 membered saturated, partially unsaturated, or aromatic fused ring having 1-4 heteroatoms independently selected from nitrogen, oxygen, and sulfur. In certain embodiments, R4 is hydrogen. In some embodiments, R4 is optionally substituted C1-6 alkyl. In certain embodiments, R4 is optionally substituted C1-3 alkyl. In certain embodiments, R4 is methyl. In certain embodiments, R4 is substituted C1-6 alkyl. In certain embodiments, R4 is benzyl. In certain embodiments, R4 is —CH2CH2N(CH3)2. In some embodiments, R4 is aryl or heteroaryl. In certain embodiments, R4 is phenyl. In some embodiments, R4 is —C(O)R, —CO2R, or —S(O)2R.

In some embodiments, R4 and R3 are taken together with their intervening atoms to form an optionally substituted 5-7 membered saturated, partially unsaturated, or aromatic fused ring having 1-4 heteroatoms independently selected from nitrogen, oxygen, and sulfur. In certain embodiments, R4 and R3 are taken together with their intervening atoms to form a 5-membered fused ring. In certain embodiments, R4 and R3 are taken together with their intervening atoms to form a fused pyrrolidine ring. In certain embodiments, R4 and R3 are taken together with their intervening atoms to form a 6-membered fused ring. In certain embodiments, R4 and R3 are taken together with their intervening atoms to form a fused piperidine ring. In certain embodiments, R4 and R3 are taken together with their intervening atoms to form a 5-7 membered partially unsaturated fused ring having 1-4 heteroatoms independently selected from nitrogen, oxygen, and sulfur. In certain embodiments, R4 and R3 are taken together with their intervening atoms to form a 5-7 membered aromatic fused ring having 1-4 heteroatoms independently selected from nitrogen, oxygen, and sulfur.

As defined generally above, R5 is R, —C(O)R, —CO2R, —C(O)N(R′)2, —C(O)C(O)R, or —C(O)CH₂C(O)R, or R5 and R2 are taken together with their intervening atoms to form an optionally substituted 5-7 membered partially unsaturated or aromatic fused ring having 1-4 heteroatoms independently selected from nitrogen, oxygen, and sulfur. In some embodiments, R5 is hydrogen. In some embodiments, R5 is optionally substituted C1-6 alkyl. In certain embodiments, R5 is methyl. In certain embodiments, R5 is substituted C1-6 alkyl. In certain embodiments, R5 is C1-6 alkyl substituted with an —OH or —OC1-6 alkyl group. In certain embodiments, R5 is —CH2CH2OCH3. In some embodiments, R4 is —C(O)R, —CO2R, —C(O)N(R′)2, —C(O)C(O)R, or —C(O)CH2C(O)R.

As defined generally above, R6 is —R, halogen, —OR, —SR, —N(R′)2, —CN, —NO2, —C(O)R, —CO2R, —C(O)N(R′)2, —C(O)SR, —C(O)C(O)R, —C(O)CH2C(O)R, —C(S)N(R′)2, —C(S)OR, —S(O)R, —SO2R, —SO2N(R′)2, —N(R′)C(O)R, —N(R′)C(O)N(R′)2, —N(R′)SO2R, —N(R′)SO2N(R′)2, —N(R′)N(R′)2, —N(R′)C(═N(R′))N(R′)2, —C═NN(R′)2, —C═NOR, —C(═N(R′))N(R′)2, —OC(O)R, or —OC(O)N(R′)2, wherein R and R′ are as defined above and described herein. In some embodiments, R6 is hydrogen. In some embodiments, R6 is optionally substituted C1-6 aliphatic. In certain embodiments, R6 is optionally substituted C1-6 alkyl, C2-6 alkenyl, or C2-6alkynyl. In certain embodiments, R6 is optionally substituted C1-6 alkyl. In certain embodiments, R6 is ethyl. In certain other embodiments, R6 is methyl, propyl, isopropyl, butyl, or isobutyl. In some embodiments, R6 is C1-6 alkyl substituted with an —OH or —OC1-6alkyl group. In certain embodiments, R6 is —CH2CH2OH or —CH2CH2OCH3. In some embodiments, R6 is cycloalkyl. In certain embodiments, R6 is cyclopropyl, cyclobutyl, cyclopentyl, or cyclohexyl. In some embodiments, R6 is optionally substituted C2-6 alkenyl. In certain embodiments, R6 is allyl. In some embodiments, R6 is optionally substituted C2-6 alkynyl. In certain embodiments, R6 is 2-propynyl. In some embodiments, R6 is optionally substituted benzyl. In certain embodiments, R6 is unsubstituted benzyl. In certain other embodiments, R6 is substituted benzyl. In some embodiments, R6 is C1-6 alkyl substituted with an ester group. In certain embodiments, R6is —CH2CO2C1-6alkyl or —CH2CO2aryl. In certain embodiments, R6 is —CH2CO2CH2CH3. In some embodiments, R6 is —OR, —SR, or —N(R′)2. In certain embodiments, R6 is —CN or —NO2. In some embodiments, R6is halogen. In certain embodiments, R6 is fluoro, chloro, bromo, or iodo. In some embodiments, R6 is —C(O)R, —CO2R, —C(O)SR, —C(O)N(R′)2, —C(O)C(O)R, or —C(O)CH2C(O)R. In certain embodiments, R6 is —C(S)OR or —C(S)N(R′)2. In other embodiments, R6 is —S(O)R, —SO2R, or —SO2N(R′)2. In some embodiments, R6 is —N(R′)C(O)R, —N(R′)C(O)N(R′)2, —N(R′)SO2R, —N(R′)SO2N(R′)2, —N(R′)N(R′)2, or —N(R′)C(═N(R′))N(R′)2. In some embodiments, R6 is —C═NN(R′)2, —C═NOR, —C(═N(R′))N(R′)2, —OC(O)R, or —OC(O)N(R′)2.

In some embodiments, R6 and R3 are taken together with their intervening atoms to form an optionally substituted 5-7 membered partially unsaturated or aromatic fused ring having 0-4 heteroatoms independently selected from nitrogen, oxygen, and sulfur. In certain embodiments, R6 and R3 are taken together with their intervening atoms to form a 5-membered fused ring. In certain embodiments, R6 and R3 are taken together with their intervening atoms to form a fused cyclopentene ring. In certain embodiments, R6 and R3 are taken together with their intervening atoms to form a 6-membered fused ring. In certain embodiments, R6 and R3 are taken together with their intervening atoms to form a fused cyclohexene ring. In certain embodiments, R6 and R3 are taken together with their intervening atoms to form a fused benzene ring. In certain embodiments, R6 and R3 are taken together with their intervening atoms to form a 5-7 membered partially unsaturated fused ring having 1-4 heteroatoms independently selected from nitrogen, oxygen, and sulfur. In certain embodiments, R6 and R3 are taken together with their intervening atoms to form a 5-7 membered aromatic fused ring having 1-4 heteroatoms independently selected from nitrogen, oxygen, and sulfur.

As defined generally above, each R is independently hydrogen or an optionally substituted group selected from C1-6 aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-3 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur. In certain embodiments, R is hydrogen. In some embodiments, R is optionally substituted C1-6 alkyl, C2-6 alkenyl, or C2-6 alkynyl. In certain embodiments, R is optionally substituted C1-6 alkyl. In certain embodiments, R is unsubstituted C1-6 alkyl. In certain embodiments, R is substituted C1-6 alkyl. In certain embodiments, R is methyl, ethyl, propyl, butyl, isopropyl, isobutyl, allyl, or benzyl.

In some embodiments, R is a 3-7 membered saturated or partially unsaturated carbocyclic ring. In certain embodiments, R is a 3-4 membered saturated carbocyclic ring. In other embodiments, R is a 5-7 membered saturated or partially unsaturated carbocyclic ring. In certain embodiments, R is cyclopropyl, cyclobutyl, cyclopentyl, cyclopentenyl, cyclohexyl, cyclohexenyl, cycloheptyl, or cycloheptenyl.

In some embodiments, R is a 4-7 membered saturated or partially unsaturated heterocyclic ring having 1-2 heteroatoms independently selected from nitrogen, oxygen, or sulfur. In certain embodiments, R is a 4-7 membered saturated heterocyclic ring. In other embodiments, R is a 5-7 membered partially unsaturated heterocyclic ring. In certain embodiments, R is tetrahydrofuranyl, tetrahydrothienyl, tetrahydropyranyl, pyrrolidinyl, pyrrolidonyl, piperidinyl, pyrrolinyl, oxazolidinyl, piperazinyl, dioxanyl, dioxolanyl, diazepinyl, oxazepinyl, thiazepinyl, or morpholinyl.

In some embodiments, R is an 8-10 membered bicyclic saturated or partially unsaturated carbocylic ring or a 7-10 membered bicyclic saturated or partially unsaturated heterocyclic ring having 1-4 heteroatoms independently selected from nitrogen, oxygen, or sulfur. In certain embodiments, R is decahydronaphthyl, tetrahydronaphthyl, or decalin. In certain other embodiments, R is tetrahydroquinolinyl, tetrahydroisoquinolinyl, or decahydroquinolinyl. In some embodiments, R is a heterocyclyl ring is fused to an aryl or heteroaryl ring. In certain embodiments, R is indolinyl, 3H-indolyl, chromanyl, phenanthridinyl, 2-azabicyclo[2.2.1]heptanyl, octahydroindolyl, or tetrahydroquinolinyl.

In some embodiments, R is phenyl or a 5-6 membered heteroaryl ring having 1-3 heteroatoms independently selected from nitrogen, oxygen, or sulfur. In certain embodiments, R is phenyl. In certain other embodiments, R is a 5-membered heteroaryl ring having 1-3 heteroatoms selected from nitrogen, oxygen, or sulfur. In yet other embodiments, R is a 6-membered heteroaryl ring having 1-3 nitrogens. In certain embodiments, R is phenyl, pyridyl, pyrimidyl, pyrazinyl, pyridazinyl, or triazinyl. In certain other embodiments, R is pyrrolyl, pyrazolyl, imidazolyl, triazolyl, tetrazolyl, thienyl, furanyl, thiazolyl, isothiazolyl, thiadiazolyl, oxazolyl, isoxazolyl, or oxadiazolyl.

In some embodiments, R is bicyclic aromatic ring. In certain embodiments, R is naphthyl. In other embodiments, R is an 8-10 membered bicyclic heteroaryl ring having 1-4 heteroatoms independently selected from nitrogen, oxygen, or sulfur. In certain embodiments, R is quinolinyl, quinoxalinyl, quinazolinyl, pyridopyrazinyl, or pyridopyrimidyl. In certain other embodiments, R is indolyl, benzimidazolyl, benzothiazolyl, benzofuranyl, benzotriazolyl, benzoxazolyl, benzothiophenyl, indazolyl, imidazopyridyl, im idazopyrim idyl, imidazopyrazinyl, imidazopyridazinyl, pyrazolopyridyl, pyrazolopyrim idyl, pyrazolopyrazinyl, pyrazolopyridazinyl, pyrrolothiazolyl, im idazo-thiazolyl, thiazolopyridyl, thiazolopyrim idyl, thiazolopypyrazinyl, thiazolopyridazinyl, oxazolopyridyl, oxazolopyrim idyl, oxazolopyrazinyl, or oxazolopyridazinyl.

As defined generally above, each R′ is independently —R, —C(O)R, —CO2R, or two R′ on the same nitrogen are taken together with the intervening nitrogen to form a 4-7 membered heterocyclic ring having 1-2 heteroatoms independently selected from nitrogen, oxygen, and sulfur. In certain embodiments, R′ is R as defined and described above. In certain embodiments, R′ is —C(O)R or —CO2R. In some embodiments, two R′ on the same nitrogen are taken together with their intervening atoms to form a 4-7 membered heterocyclic ring having 1-2 heteroatoms independently selected from nitrogen, oxygen, and sulfur. In certain embodiments, two R′ on the same nitrogen are taken together with their intervening atoms to form an azetidine, pyrrolidine, piperidine, morpholine, piperazine, homopiperidine, or homopiperazine ring.

According to one aspect, a provided compound is of formula:

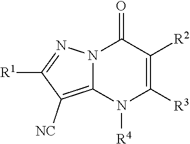

or a pharmaceutically acceptable salt thereof, wherein R1, R2, R3, and R4 are as defined and described herein. In certain embodiments, a compound of formula II has one of the following formulae:

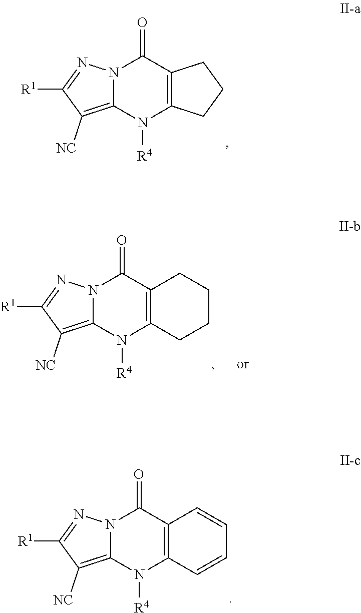

According to another aspect, a provided compound is of formula III:

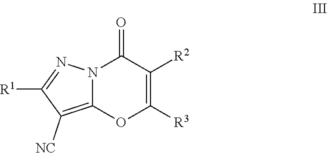

or a pharmaceutically acceptable salt thereof, wherein R1, R2, R3, and R4 are as defined and described herein. In certain embodiments, a compound of formula II has one of the following formulae:

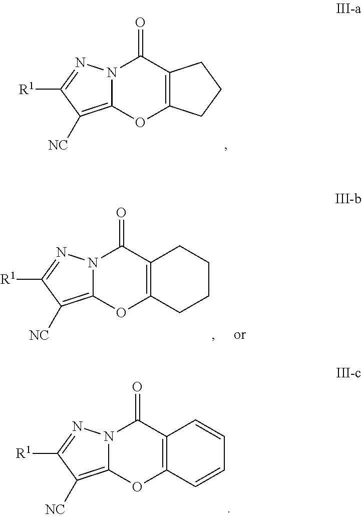

According to another aspect, a provided compound is of formula:

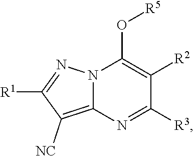

or a pharmaceutically acceptable salt thereof, wherein R1, R2, R3, and R5 are as defined and described herein. In certain embodiments, R5 is optionally substituted C1-6 aliphatic. In certain embodiments, R5 is methyl. In some embodiments, R5 is optionally substituted C1-6 alkyl. In certain embodiments, R5 is substituted C1-6 alkyl. In certain embodiments, R5 is C1-6alkyl substituted with —OH or —OC1-6alkyl. In certain embodiments, R5 is —CH2CH2OMe.

According to another aspect, a provided compound is of formula:

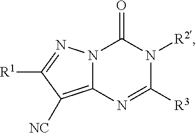

or a pharmaceutically acceptable salt thereof, wherein R1, R2′, and R3 are as defined and described herein.

Exemplary compounds that can be used as anti-PESC agents are set forth in Table 1 below.

TABLE 1

| | |
|---|---|
| 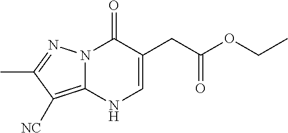 [SMILES: CCOC(=O)Cc1c[nH]c2c(C#N)c(C)nn2c1=O] | I-1 |
| 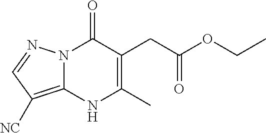 [SMILES: CCOC(=O)Cc1c(C)[nH]c2c(C#N)cnn2c1=O] | I-2 |
| 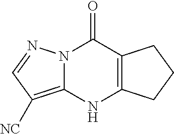 [SMILES: N#Cc1cnn2c(=O)c3c([nH]c12)CCC3] | I-3 |
| 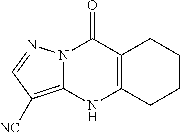 [SMILES: N#Cc1cnn2c(=O)c3c([nH]c12)CCCC3] | I-4 |
| 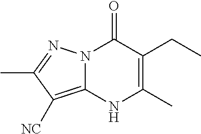 [SMILES: CCc1c(C)[nH]c2c(C#N)c(C)nn2c1=O] | I-5 |
| 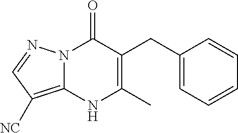 [SMILES: Cc1[nH]c2c(C#N)cnn2c(=O)c1Cc1ccccc1] | I-6 |
| 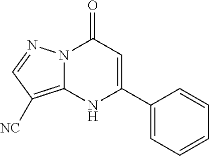 [SMILES: N#Cc1cnn2c(=O)cc(-c3ccccc3)[nH]c12] | I-7 |
| 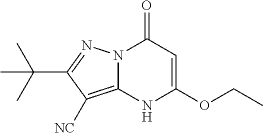 [SMILES: CCOc1cc(=O)n2nc(C(C)(C)C)c(C#N)c2[nH]1] | I-8 |
| 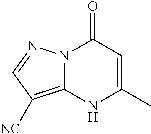 | I-9 |
| 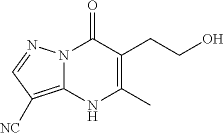 [SMILES: Cc1[nH]c2c(C#N)cnn2c(=O)c1CCO] | I-10 |
| 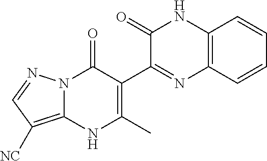 [SMILES: Cc1[nH]c2c(C#N)cnn2c(=O)c1-c1nc2ccccc2[nH]c1=O] | I-11 |
| 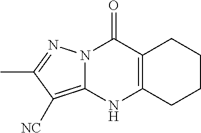 [SMILES: Cc1nn2c(=O)c3c([nH]c2c1C#N)CCCC3] | I-12 |
| 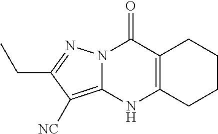 [SMILES: CCc1nn2c(=O)c3c([nH]c2c1C#N)CCCC3] | I-13 |
| 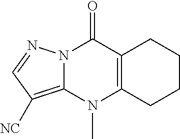 [SMILES: Cn1c2c(c(=O)n3ncc(C#N)c13)CCCC2] | I-14 |
| 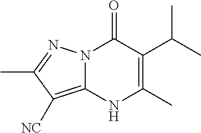 [SMILES: Cc1nn2c(=O)c(C(C)C)c(C)[nH]c2c1C#N] | I-15 |
| 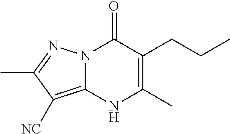 [SMILES: CCCc1c(C)[nH]c2c(C#N)c(C)nn2c1=O] | I-16 |
| 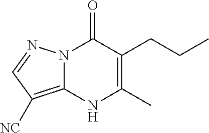 [SMILES: CCCc1c(C)[nH]c2c(C#N)cnn2c1=O] | I-17 |
| 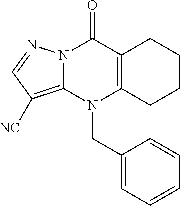 [SMILES: N#Cc1cnn2c(=O)c3c(n(Cc4ccccc4)c12)CCCC3] | I-18 |
| 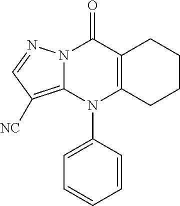 [SMILES: N#Cc1cnn2c(=O)c3c(n(-c4ccccc4)c12)CCCC3] | I-19 |
| 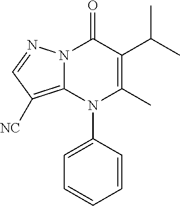 [SMILES: Cc1c(C(C)C)c(=O)n2ncc(C#N)c2n1-c1ccccc1] | I-20 |
| 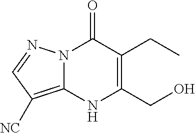 [SMILES: CCc1c(CO)[nH]c2c(C#N)cnn2c1=O] | I-21 |
| 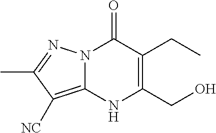 [SMILES: CCc1c(CO)[nH]c2c(C#N)c(C)nn2c1=O] | I-22 |
| 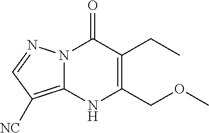 [SMILES: CCc1c(COC)[nH]c2c(C#N)cnn2c1=O] | I-23 |
| 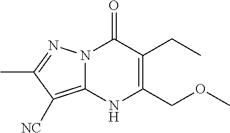 | I-24 |
| 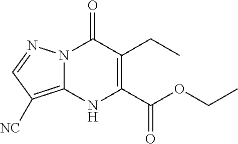 [SMILES: CCOC(=O)c1[nH]c2c(C#N)cnn2c(=O)c1CC] | I-25 |
| 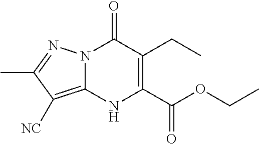 [SMILES: CCOC(=O)c1[nH]c2c(C#N)c(C)nn2c(=O)c1CC] | I-26 |
| 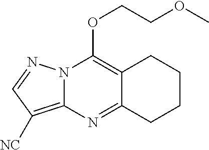 [SMILES: COCCOc1c2c(nc3c(C#N)cnn13)CCCC2] | I-27 |
| 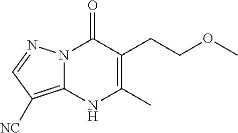 [SMILES: COCCc1c(C)[nH]c2c(C#N)cnn2c1=O] | I-28 |
| 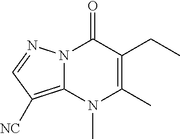 [SMILES: CCc1c(C)n(C)c2c(C#N)cnn2c1=O] | I-29 |
| 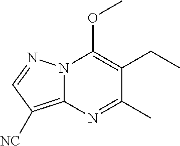 [SMILES: CCc1c(C)nc2c(C#N)cnn2c1OC] | I-30 |
| 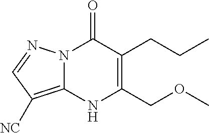 [SMILES: CCCc1c(COC)[nH]c2c(C#N)cnn2c1=O] | I-31 |
| 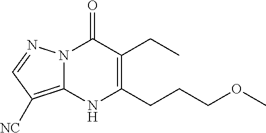 [SMILES: CCc1c(CCCOC)[nH]c2c(C#N)cnn2c1=O] | I-32 |
| 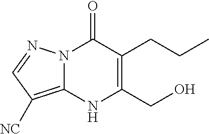 [SMILES: CCCc1c(CO)[nH]c2c(C#N)cnn2c1=O] | I-33 |
| 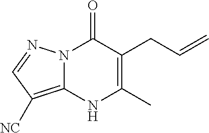 [SMILES: C=CCc1c(C)[nH]c2c(C#N)cnn2c1=O] | I-34 |
| 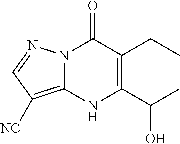 [SMILES: CCc1c(C(C)O)[nH]c2c(C#N)cnn2c1=O] | I-35 |
| 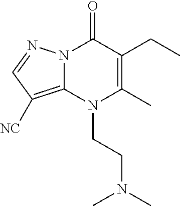 [SMILES: CCc1c(C)n(CCN(C)C)c2c(C#N)cnn2c1=O] | I-36 |
| 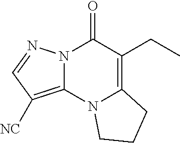 [SMILES: CCc1c2n(c3c(C#N)cnn3c1=O)CCC2] | I-37 |
| 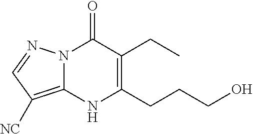 [SMILES: CCc1c(CCCO)[nH]c2c(C#N)cnn2c1=O] | I-38 |
| 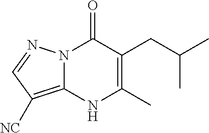 [SMILES: Cc1[nH]c2c(C#N)cnn2c(=O)c1CC(C)C] | I-39 |
| 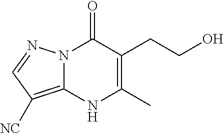 [SMILES: Cc1[nH]c2c(C#N)cnn2c(=O)c1CCO] | I-40 |
| 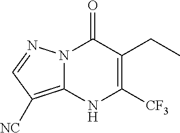 [SMILES: CCc1c(C(F)(F)F)[nH]c2c(C#N)cnn2c1=O] | I-41 |
| 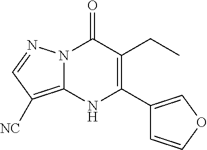 [SMILES: CCc1c(-c2ccoc2)[nH]c2c(C#N)cnn2c1=O] | I-42 |
| 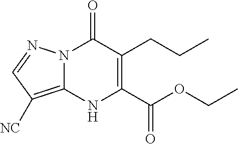 [SMILES: CCCc1c(C(=O)OCC)[nH]c2c(C#N)cnn2c1=O] | I-43 |
| 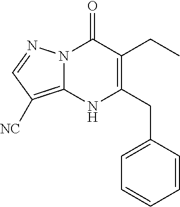 [SMILES: CCc1c(Cc2ccccc2)[nH]c2c(C#N)cnn2c1=O] | I-44 |
| 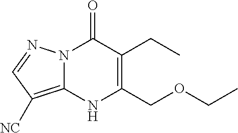 [SMILES: CCOCc1[nH]c2c(C#N)cnn2c(=O)c1CC] | I-45 |
| 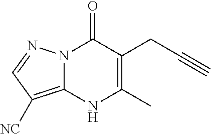 [SMILES: C#CCc1c(C)[nH]c2c(C#N)cnn2c1=O] | I-46 |
| 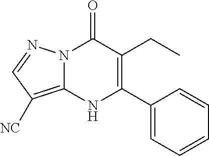 [SMILES: CCc1c(-c2ccccc2)[nH]c2c(C#N)cnn2c1=O] | I-47 |
| 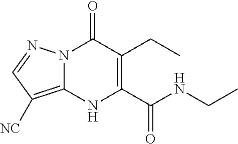 [SMILES: CCNC(=O)c1[nH]c2c(C#N)cnn2c(=O)c1CC] | I-48 |
| 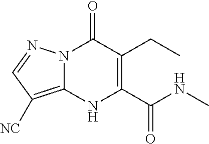 [SMILES: CCc1c(C(=O)NC)[nH]c2c(C#N)cnn2c1=O] | I-49 |
| 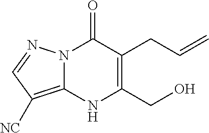 [SMILES: C=CCc1c(CO)[nH]c2c(C#N)cnn2c1=O] | I-50 |
| 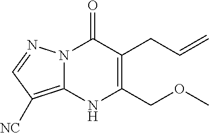 [SMILES: C=CCc1c(COC)[nH]c2c(C#N)cnn2c1=O] | I-51 |
| 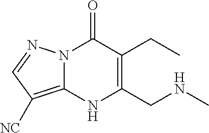 [SMILES: CCc1c(CNC)[nH]c2c(C#N)cnn2c1=O] | I-52 |
| 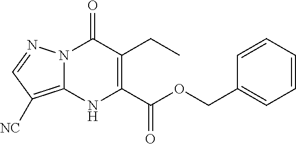 [SMILES: CCc1c(C(=O)OCc2ccccc2)[nH]c2c(C#N)cnn2c1=O] | I-53 |
| 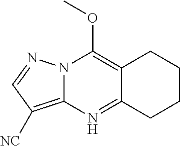 [SMILES: COc1c2c(nc3c(C#N)cnn13)CCCC2] | I-54 |
| 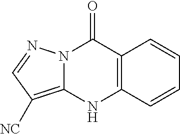 [SMILES: N#Cc1cnn2c(=O)c3ccccc3[nH]c12] | I-55 |
| 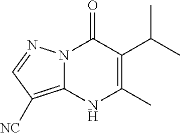 [SMILES: Cc1[nH]c2c(C#N)cnn2c(=O)c1C(C)C] | I-56 |
| 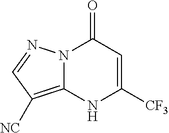 [SMILES: N#Cc1cnn2c(=O)cc(C(F)(F)F)[nH]c12] | I-57 |
| 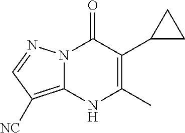 [SMILES: Cc1[nH]c2c(C#N)cnn2c(=O)c1C1CC1] | I-58 |
| 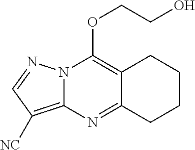 [SMILES: N#Cc1cnn2c(OCCO)c3c(nc12)CCCC3] | I-59 |
| 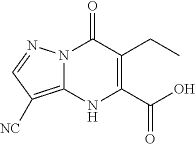 [SMILES: CCc1c(C(=O)O)[nH]c2c(C#N)cnn2c1=O] | I-60 |
| 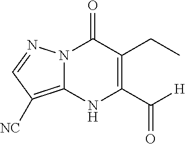 [SMILES: CCc1c(C=O)[nH]c2c(C#N)cnn2c1=O] | I-61 |
| 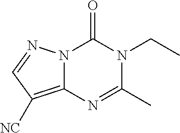 | I-62 |
| 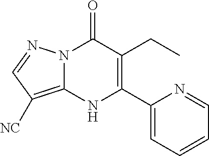 [SMILES: CCc1c(-c2ccccn2)[nH]c2c(C#N)cnn2c1=O] | I-63 |
| 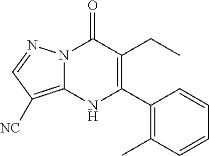 [SMILES: CCc1c(-c2ccccc2C)[nH]c2c(C#N)cnn2c1=O] | I-64 |
| 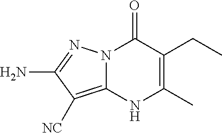 [SMILES: CCc1c(C)[nH]c2c(C#N)c(N)nn2c1=O] | I-65 |
| 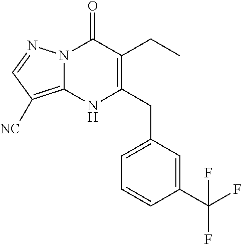 [SMILES: CCc1c(Cc2cccc(C(F)(F)F)c2)[nH]c2c(C#N)cnn2c1=O] | I-66 |
| 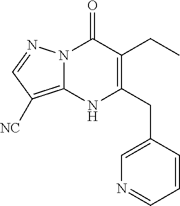 [SMILES: CCc1c(Cc2cccnc2)[nH]c2c(C#N)cnn2c1=O] | I-67 |
| 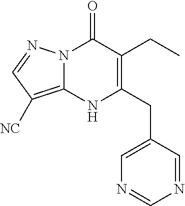 [SMILES: CCc1c(Cc2cncnc2)[nH]c2c(C#N)cnn2c1=O] | I-68 |
| 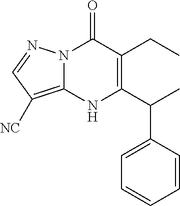 [SMILES: CCc1c(C(C)c2ccccc2)[nH]c2c(C#N)cnn2c1=O] | I-69 |
| 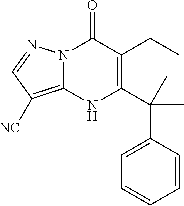 [SMILES: CCc1c(C(C)(C)c2ccccc2)[nH]c2c(C#N)cnn2c1=O] | I-70 |
| 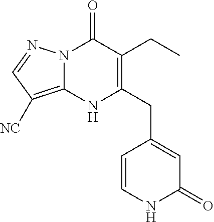 [SMILES: CCc1c(Cc2cc[nH]c(=O)c2)[nH]c2c(C#N)cnn2c1=O] | I-71 |
| 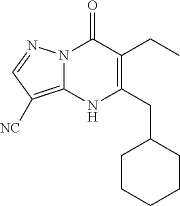 [SMILES: CCc1c(CC2CCCCC2)[nH]c2c(C#N)cnn2c1=O] | I-72 |
| 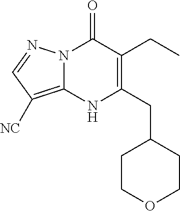 [SMILES: CCc1c(CC2CCOCC2)[nH]c2c(C#N)cnn2c1=O] | I-73 |

In certain embodiments, the present disclosure provides any compound depicted in Table 1, above, or a pharmaceutically acceptable salt thereof.

In other embodiments, the present disclosure provides a compound of formula:

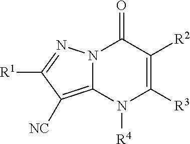

or a salt thereof, wherein:

R1 is H, C1-6alkyl, trifluoromethyl, 3-6 membered carbocyclyl, 6 membered aryl, 3-6 membered heterocyclyl, 5-6 membered heteroaryl, halo, —ORf, —SRf, —N(Rf)2, —CN, or —NO2, wherein said alkyl, carbocyclyl, aryl, heteroaryl, and heterocyclyl are optionally substituted with one or more groups independently selected from oxo, halo, C1-3alkoxy and C1-3alkyl;

R2 and R3 are each independently H, C1-12alkyl, C2-12alkenyl, C2-12alkynyl, carbocyclyl, aryl, heterocyclyl, heteroaryl, halo, —ORa, —SRa, —N(Ra)2, —CN, —NO2, —C(O)Ra, —CO2Ra, —C(O)N(Ra)2, —C(O)SRa, —C(O)C(O)Ra, —C(O)CH2C(O)Ra, —C(S)N(Ra)2, —C(S)ORa, —S(O)Ra, —SO2Ra, —SO2N(Ra)2, —N(Ra)C(O)Ra, —N(Ra)C(O)N(Ra)2, —N(Ra)SO2Ra, —N(Ra)SO2N(Ra)2, —N(Ra)N(Ra)2, —N(Ra)C(═N(Ra))N(Ra)2, —C(═N)N(Ra)2, —C═NORa, —C(═N(Ra))N(Ra)2, —OC(O)Ra, or —OC(O)N(Ra)2, wherein each C1-12alkyl, C2-12alkenyl, C2-12alkynyl, carbocyclyl, aryl, heteroaryl, and heterocyclyl of R2 and R3 is independently optionally substituted with one or more groups Rx; and wherein R2 and R3 are not each H; or R2 and R3 taken together with the atoms to which they are attached form a 4, 5, 6, 7, or 8 membered carbocyclyl or aryl, which carbocyclyl or aryl is optionally substituted with one or more groups Rx;

R4 is H, C1-12 alkyl, C2-12 alkenyl, C2-12 alkynyl, carbocyclyl, aryl, heteroaryl, and heterocyclyl, wherein each C1-12 alkyl, C2-12 alkenyl, C2-12 alkynyl, carbocyclyl, aryl, heteroaryl, and heterocyclyl is optionally substituted with one or more groups independently selected from oxo, C1-12 alkyl, C1-12 haloalkyl, carbocyclyl, aryl, heterocyclyl, heteroaryl, halo, —CN, —NO2, —NRmRm, —ORm, —C(═O)ORm, and —OC(═O)Rm; or R4 and R3 taken together with the atoms to which they are attached form a heterocyclyl;

each Ra is independently selected from H, C1-6 alkyl, C2-6 alkenyl, C2-6 alkynyl, carbocyclyl, aryl, heteroaryl, and heterocyclyl, wherein each C1-6 alkyl, C2-6 alkenyl, C2-6 alkynyl, carbocyclyl, aryl, heteroaryl, and heterocyclyl is optionally substituted with one or more groups Rx;

each Rf is independently selected from H, C1-3 alkyl, trifluoromethyl, 3-6 membered carbocyclyl, 6 membered aryl, 3-6 membered heterocyclyl, and 5-6 membered heteroaryl, or two Rf groups together with the nitrogen to which they are attached form a 3-6 membered heterocycle;

each Rg is independently selected from H, C1-6 alkyl, C2-6 alkenyl, C2-6 alkynyl, C3-8 carbocyclyl, aryl, heteroaryl, and heterocyclyl, wherein each C1-6 alkyl, C2-6 alkenyl, C2-6 alkynyl, C3-8 carbocyclyl, aryl, heteroaryl, and heterocyclyl is optionally substituted with one or more groups Rx; or two Rg groups together with the nitrogen to which they are attached form a 3-6 membered heterocycle or a 5-6 membered heteroaryl;

each Rm is independently selected from H, C1-6 alkyl, C2-6 alkenyl, C2-6 alkynyl, C1-6 haloalkyl, carbocyclyl, C1-6 alkanoyl, phenyl, and benzyl, wherein any C1-6 alkyl, C2-6 alkenyl, C2-6 alkynyl, C1-6 haloalkyl, carbocyclyl, C1-6 alkanoyl, phenyl, or benzyl is optionally substituted with one or more groups independently selected from halo, —CN, —NO2, —NRyRz, and —ORw; or two Rm groups together with the nitrogen to which they are attached form a 3-6 membered heterocycle;

each Rv is independently hydrogen, C1-6 alkyl, C2-6 alkenyl, C2-6 alkynyl, carbocyclyl, aryl, heteroaryl, and heterocyclyl, wherein each C1-6 alkyl, C2-6 alkenyl, C2-6 alkynyl, carbocyclyl, aryl, heteroaryl, and heterocyclyl is optionally substituted with one or more groups independently selected from oxo, halo, amino, hydroxyl, aryl, carbocyclyl, and C1-C6 alkyl that is optionally substituted with one or more groups independently selected from oxo and halo; or two Rv are taken together with the nitrogen to which they are attached to form a heterocyclyl that is optionally substituted with one or more groups independently selected from oxo, halo and C1-3 alkyl that is optionally substituted with one or more groups independently selected from oxo and halo;

each Rw is independently selected from H, C1-4 alkyl, C1-4 alkanoyl, phenyl, benzyl, and phenethyl;

each Rx is independently selected from oxo, C1-6 alkyl, C2-6 alkenyl, C2-6 alkynyl, carbocyclyl, aryl, heteroaryl, heterocycle, —F, —Cl, —Br, —I, —NO2, —N(Rv)2, —CN, —C(O)—N(Rv)2, —S(O)—N(Rv)2, —S(O)2-N(Rv)2, —O—Rv, —S—Rv, —O—C(O)—Rv, —O—C(O)—O—Rv, —C(O)—Rv, —C(O)—O—Rv, —S(O)—Rv, —S(O)2-Rv, —O—C(O)—N(Rv)2, —N(Rv)-C(O)—ORv, —N(Rv)-C(O)—N(Rv)2, —S(O)2-N(Rv)2, —N(Rv)-C(O)—Rv, —N(Rv)-S(O)—Rv, —N(Rv)-S(O)2-Rv, —N(Rv)-S(O)—N(Rv)2, and

—N(Rv)-S(O)2-N(Rv)2, wherein any C1-6 alkyl, C2-6 alkenyl, C2-6 alkynyl, carbocyclyl, aryl, heteroaryl, and heterocycle is optionally substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, —N(Rv)-S(O)2-Rv, and C1-6 alkyl that is optionally substituted with one or more groups independently selected from oxo and halo;

each Ry and Rz is independently selected from H, C1-4 alkyl, C1-4 alkanoyl, C1-4 alkoxycarbonyl, phenyl, benzyl, and phenethyl, or Ry and Rz together with the nitrogen to which they are attached form a heterocyclyl;

each Rxa is independently selected from aryl, heteroaryl, heterocycle, and carbocycle, wherein any aryl, heteroaryl, heterocycle, and carbocycle is optionally substituted with one or more groups independently selected from C1-6 alkyl, C2-6 alkenyl, C2-6 alkynyl, —F, —Cl, —Br, —I, —NO2, —N(Rv)2, —CN, carbocycle, aryl, —C(O)—N(Rv)2, —S(O)—N(Rv)2, —S(O)2-N(Rv)2, —O—Rv, —S—Rv, —O—C(O)—Rv, —O—C(O)—O—Rv, —C(O)—Rv, —C(O)—O—Rv, —S(O)—Rv, —S(O)2-Rv, —O—C(O)—N(Rv)2, —N(Rv)-C(O)—ORv, —N(Rv)-C(O)—N(Rv)2, —S(O)2-N(Rv)2, —N(Rv)-C(O)—Rv, —N(Rv)-S(O)—Rv, —N(Rv)-S(O)2-Rv, and —N(Rv)-S(O)—N(Rv)2, wherein any C1-6 alkyl, C2-6 alkenyl, and C2-6 alkynyl is optionally substituted with one or more groups independently selected from oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-S(O)—Rv, and —N(Rv)-S(O)2-Rv.

In certain embodiments, R1 is H, C1-6 alkyl, trifluoromethyl, 3-6 membered carbocyclyl, 6 membered aryl, 3-6 membered heterocyclyl, 5-6 membered heteroaryl, halo, —ORf, —SRf, —N(Rf)2, —CN, or —NO2, wherein said alkyl, carbocyclyl, aryl, heteroaryl, and heterocyclyl are optionally substituted with one or more groups independently selected from oxo, halo, C1-3 alkoxy and C1-3 alkyl.

In certain embodiments, R1 is H, methyl, or ethyl.

In certain embodiments, R1 is H.

In certain embodiments, R2 is H.

In certain embodiments, R2 is C1-12 alkyl, C2-12 alkenyl, C2-12 alkynyl, carbocyclyl, aryl, heterocyclyl, heteroaryl, halo, —ORa, —SRa, —N(Ra)2, —CN, —NO2, —C(O)Ra, —CO2Ra, —C(O)N(Ra)2, —C(O)SRa, —C(O)C(O)Ra, —C(O)CH₂C(O)Ra, —C(S)N(Ra)2, —C(S)ORa, —S(O)Ra, —SO2Ra, —SO2N(Ra)2, —N(Ra)C(O)Ra, —N(Ra)C(O)N(Ra)2, —N(Ra)SO2Ra, —N(Ra)SO2N(Ra)2, —N(Ra)N(Ra)2, —N(Ra)C(═N(Ra))N(Ra)2, —C(═N)N(Ra)2, —C═NORa, —C(═N(Ra))N(Ra)2, —OC(O)Ra, or —OC(O)N(Ra)2, wherein each C1-12 alkyl, C2-12 alkenyl, C2-12 alkynyl, carbocyclyl, aryl, heteroaryl, and heterocyclyl of R2is independently optionally substituted with one or more groups Rx.

In certain embodiments, R2 and R3 taken together with the atoms to which they are attached form a 4, 5, 6, 7, or 8 membered carbocyclyl or aryl, which carbocyclyl or aryl is optionally substituted with one or more groups Rx.

In certain embodiments, R2 is H, C1-6 alkyl, C2-12 alkenyl, C2-12 alkynyl, carbocyclyl, aryl, heteroaryl, halo, —CN, —SRa, —N(Rv)2, and —CO2Ra, wherein any C1-6 alkyl, carbocyclyl and aryl is optionally substituted with one or more groups independently selected from C1-3alkyl, carbocyclyl, halo, —CN, —N(Rv)-C(O)—Rv, and —O—Rv.

In certain embodiments, R2 is H, isopropyl, ethyl, tert-butyl, 2,2-difluoroethyl, cyclobutyl, 2-propyn-1-yl, bromo, chloro, 2-furyl, vinyl, phenyl, 2-chlorophenylthio, 2-fluoroethyl, 2-propenyl, 1-methylvinylcyclopropyl, 4-pyridyl, 2-buten-1-yl, iodo, 1-methyl-2-propyn-1-yl, 1-methylprop-1-yl, 1-(cyclopropyl)ethyl, methoxycarbonyl, 2-butynyl, 2-hydroxy-1-methylethyl, 4-(methylcarbonylamino)butyl, 3-(methylcarbonylamino)propyl, 4-aminobutyl, 1-methyl-2-propenyl, 1-methylcyclobutyl, propyl, 2-methoxyethyl, and 2-methylpropyl.

In certain embodiments, R3 is H.

In certain embodiments, R3 is C1-12 alkyl, C2-12 alkenyl, C2-12 alkynyl, carbocyclyl, aryl, heterocyclyl, heteroaryl, halo, —ORa, —SRa, —N(Ra)2, —CN, —NO2, —C(O)Ra, —CO2Ra, —C(O)N(Ra)2, —C(O)SRa, —C(O)C(O)Ra, —C(O)CH₂C(O)Ra, —C(S)N(Ra)2, —C(S)ORa, —S(O)Ra, —SO2Ra, —SO2N(Ra)2, —N(Ra)C(O)Ra, —N(Ra)C(O)N(Ra)2, —N(Ra)SO2Ra, —N(Ra)SO2N(Ra)2, —N(Ra)N(Ra)2, —N(Ra)C(═N(Ra))N(Ra)2, —C(═N)N(Ra)2, —C═NORa, —C(═N(Ra))N(Ra)2, —OC(O)Ra, or —OC(O)N(Ra)2, wherein each C1-12 alkyl, C2-12 alkenyl, C2-12 alkynyl, carbocyclyl, aryl, heteroaryl, and heterocyclyl of R3is independently optionally substituted with one or more groups Rx.

In certain embodiments, R3 is H, C1-12 alkyl, C2-12 alkenyl, C2-12 alkynyl, aryl, heterocyclyl, heteroaryl, halo, —ORa, —N(Ra)2, —C(O)Ra, —CO2Ra, —C(O)N(Ra)2, or —N(Ra)C(O)Ra, wherein each C1-12 alkyl, C2-12 alkenyl, C2-12 alkynyl, aryl, heteroaryl, and heterocyclyl of R3 is independently optionally substituted with one or more groups Rx.

In certain embodiments, R3 is H, methyl, chloro, bromo, carboxy, formyl, aminocarbonyl, furan-3-yl, phenyl, benzyl, phenethyl, phenoxy, 1H-pyrazol-4-yl, 1-(cyclopropylmethyl)-1H-pyrazol-4-yl, 1-(1-methylcyclopropyl)-1H-pyrazol-4-yl, 5-fluoro-1H-pyrazol-4-yl, 1-(2-phenylpropan-2-yl)-1H-pyrazol-4-yl, 1-(pyridin-3-yl)-1H-pyrazol-4-yl, 1-(pyridin-4-yl)-1H-pyrazol-4-yl, 1-(pyridin-2-yl)-1H-pyrazol-4-yl, 1-[1-(N-methylaminocarbonyl)-1,1-dimethylmethyl]-1H-pyrazol-4-yl, 5-fluoro-1-isopropyl-1H-pyrazol-4-yl, 1-(cyclopropyl-methyl)-1H-pyrazol-5-yl, 1-(cyclopropylmethyl)-1H-pyrazol-3-yl, 1-(tetrahydro-2H-thio-pyran-4-yl)-1H-pyrazol-4-yl, 1-(1,1-dioxidotetrahydro-2H-thiopyran-4-yl)-1H-pyrazol-4-yl, 1-((6-(3-oxobut-1-en-1-yl)pyridin-2-yl)methyl)-1H-pyrazol-4-yl, 3-iodophenyl, methyl-aminocarbonyl, 3-methyl-1,2,4-oxadiazol-5-yl, 5-methyl-1,3,4-oxadiazol-2-yl, 1H-imidazol-2-yl, N-(benzoylmethyl)aminocarbonyl, 5-phenyloxazol-2-yl, 1-cyclohexyl-pyrazol-4-yl, 1-isopropylpyrazol-4-yl, biphenyl-3-yl, 3-((4-fluorophenyl)amino)phenyl, 3-(2-oxopyrrolidin-1-yl)phenyl, 3-(methylcarbonylamino)-5-phenylphenyl, phenylamino, piperidin-1-yl, methoxymethyl, ethoxymethyl, ethoxycarbonyl, 3-methoxypropyl, benzyl-oxycarbonyl, trifluoromethyl, 3-furyl, ethylaminocarbonyl, hydroxymethyl, 3-hydroxypropyl, 2-hydroxyethyl, methylaminomethyl, benzofuran-3-yl, 1-phenyl-1H-pyrazol-3-yl, 5-cyclopropylfuran-2-yl, 2-methylfuran-3-yl, 1-phenyl-1H-pyrazol-4-yl, 1-ethyl-1H-pyrazol-4-yl, 1-methyl-6-oxo-1,6-dihydropyridin-3-yl, furan-2-yl, 5-phenylfuran-2-yl, 1-isopropyl-1H-pyrazol-4-yl, pyrimidin-5-yl, 5-methylpyridin-3-yl, 1-methyl-1H-pyrazol-3-yl, 4-phenylfuran-2-yl, 2-fluorophenyl, 4-cyanophenyl, 4-methoxyphenyl, 4-(trifluoromethyl)phenyl, 4-fluorophenyl, 1-benzyl-1H-pyrazol-4-yl, 5-chloropyridin-3-yl, 5-fluoropyridin-3-yl, prop-1-en-2-yl, vinyl, 1-methyl-1H-pyrazol-5-yl, 4-(hydroxymethyl)-furan-2-yl, 3-cyanophenyl, 1H-pyrazol-5-yl, 2,5-dihydrofuran-3-yl, thiophen-3-yl, thiophen-2-yl, 1-methyl-1H-pyrazol-4-yl, 5-methylfuran-2-yl, 5-(hydroxymethyl)furan-2-yl, 3-(trifluoromethyl)phenyl, 3-methoxyphenyl, 3-fluorophenyl, pyridin-3-yl, 1-(methylsulfonyl)-1H-pyrazol-4-yl, 1-cyclopentyl-1H-pyrazol-4-yl, 1-(thiophen-3-ylmethyl)-1H-pyrazol-4-yl, 4-chloro-3-(morpholine-4-carbonyl)phenyl, 3-chloro-4-(cyclopropyl-aminocarbonyl)phenyl, 1-(1-hydroxy-2-methylpropan-2-yl)-1H-pyrazol-4-yl, 1-(3-methoxybenzyl)-1H-pyrazol-4-yl, 1-(pyridin-4-ylmethyl)-1H-pyrazol-4-yl, 1-(2-chloro-benzyl)-1H-pyrazol-4-yl, 1-(3-phenoxybenzyl)-1H-pyrazol-4-yl, 1-(4-phenoxybenzyl)-1H-pyrazol-4-yl, 1-cyclohexyl-1H-pyrazol-4-yl, 1-(1-phenylethyl)-1H-pyrazol-4-yl, 1-cyclobutyl-1H-pyrazol-4-yl, 1-(sec-butyl)-1H-pyrazol-4-yl, 4-fluoro-3-(pyrrolidine-1-carbonyl)phenyl, 1-(cyclopropylsulfonyl)-1H-pyrazol-3-yl, 1-(cyclopropanecarbonyl)-1H-pyrazol-3-yl, 1-(2-cyclopropylethyl)-1H-pyrazol-4-yl, 1-([1,1′-biphenyl]-3-ylmethyl)-1H-pyrazol-4-yl, 1-phenethyl-1H-pyrazol-4-yl, 1-(2-methoxybenzyl)-1H-pyrazol-4-yl, 1-(4-methoxybenzyl)-1H-pyrazol-4-yl, 1-(tert-butyl)-1H-pyrazol-4-yl, 3,4-dimethylphenyl, 3-chloro-4-ethoxyphenyl, 4-methoxy-3-methylphenyl, 2-methylbenzo[d]thiazol-5-yl, 1-(2-phenoxybenzyl)-1H-pyrazol-4-yl, 1-(phenylsulfonyl)-1H-pyrazol-4-yl, 1-benzoyl-1H-pyrazol-4-yl, 1-benzhydryl-1H-pyrazol-4-yl, 1-([1,1′-biphenyl]-2-ylmethyl)-1H-pyrazol-4-yl, 1-(cyclohexylmethyl)-1H-pyrazol-4-yl, 1-(pyridin-3-ylmethyl)-1H-pyrazol-4-yl, benzo-furan-2-yl, (E)-styryl, 5-ethylfuran-2-yl, 1-(2-methoxyethyl)-1H-pyrazol-4-yl, 1-(naphthalen-1-ylmethyl)-1H-pyrazol-4-yl, 1-([1, 1′-biphenyl]-4-ylmethyl)-1H-pyrazol-4-yl, 3-phenoxyphenyl, phenylethynyl, 3,4-dichlorophenyl, 3-chloro-4-methoxyphenyl, 3-methoxy-4-methylphenyl, 1-(thiazol-4-ylmethyl)-1H-pyrazol-4-yl, 1H-indazol-5-yl, 3,4-dimethoxyphenyl, 4-methoxy-3,5-dimethylphenyl, 1-(oxetan-3-yl)-1H-pyrazol-4-yl, 1-(2-fluorobenzyl)-1H-pyrazol-4-yl, 1-(4-fluorobenzyl)-1H-pyrazol-4-yl, 1-(methoxy-carbonylmethyl)-1H-pyrazol-4-yl, 1-(2-(dimethylamino)ethyl)-1H-pyrazol-4-yl, 3-cyano-4-methylphenyl, benzo[d][1,3]dioxol-5-yl, 2,3-dihydrobenzofuran-5-yl, 1-(3-fluorobenzyl)-1H-pyrazol-4-yl, 1-(thiophen-2-ylmethyl)-1H-pyrazol-4-yl, 1-(2,2,2-trifluoroethyl)-1H-pyrazol-4-yl, 1-(3-chlorobenzyl)-1H-pyrazol-4-yl, 1-isobutyl-1H-pyrazol-4-yl, 1-(3,3,3-trifluoropropyl)-1H-pyrazol-4-yl, 1-(difluoromethyl)-1H-pyrazol-4-yl, 1-(2-cyanoethyl)-1H-pyrazol-4-yl, 4-cyclopropylfuran-2-yl, 1H-pyrrol-3-yl, 2,2-difluorobenzo[d][1,3]dioxol-5-yl, 3-fluoro-4-(aminocarbonyl)phenyl, 3-fluoro-4-(methylsulfonyl)phenyl, 3-chloro-4-(trifluoromethoxy)phenyl, 5-fluoro-3-(aminocarbonyl)phenyl, 3-(hydroxymethyl)-4-methoxyphenyl, 1-(methylsulfonyl)-1H-pyrrol-3-yl, 1-methyl-1H-pyrrol-3-yl, 1H-indol-2-yl, cyclopropylcarbonylamino, benzoylamino, 3-bromophenyl, 3-(1-methylpyrazol-4-yl)phenyl, 3-(1-isopropylpyrazol-4-yl)phenyl, 4-phenylphenyl, 4-(4-fluoroanilino)phenyl, 3-(tert-butoxycarbonylamino)phenyl, 1-acetyl-1,2,3,6-tetrahydropyridin-4-yl, 1-propionyl-1,2,3,6-tetrahydropyridin-4-yl, 1-acryloyl-1,2,3,6-tetrahydropyridin-4-yl, 1-methyl-1,2,3,6-tetrahydropyridin-4-yl, 1-((2-methylthiazol-4-yl)methyl)-1H-pyrazol-4-yl, 1-(2-(acetyl-amino)ethyl)-1H-pyrazol-4-yl, 3,5-dichlorophenyl, 2-fluoro-4-(methylsulfonyl)phenyl, 1-(tert-pentyl)-1H-pyrazol-4-yl, 3-(2-morpholinoethyl)phenyl, 3-(2-(dimethyl-amino)ethyl)-phenyl, 1-(1-(thiazol-4-yl)ethyl)-1H-pyrazol-4-yl, 1-(tetrahydro-2H-pyran-4-yl)-1H-pyrazol-4-yl, 3-methoxy-4-(trifluoromethyl)phenyl, 3-methoxycarbonyl-4-chlorophenyl, 4-(trifluoromethoxy)phenyl, 3-methyl-4-(trifluoromethoxy)phenyl, 4-cyclopropyl-3-(trifluoro-methyl)phenyl, 2,2-dimethyl-2,3-dihydrobenzofuran-5-yl, 3,5-dimethoxyphenyl, 3,4-difluorophenyl, 4-biphenyl, 3-chloro-5-fluorophenyl, 3,5-bis(trifluoromethyl)phenyl, 3-fluoro-5-methoxyphenyl, 3-(aminocarbonyl)phenyl, 4-(cyclopropylmethoxy)phenyl, 2-fluoro-5-(benzyloxycarbonyl)phenyl, 3-(1H-pyrazol-1-yl)phenyl, 1-(2-hydroxycyclo-pentyl)-1H-pyrazol-4-yl, 3-(N-methylaminosulfonyl)phenyl, 4-(2-hydroxypropan-2-yl)-phenyl, 2-(trifluoromethyl)pyridin-4-yl, 6-phenoxypyridin-3-yl, 2-methoxypyridin-4-yl, 4-methyl-2-phenylthiazol-5-yl, 3-amino-5-cyanophenyl, 1-(tetrahydrofuran-3-yl, 3-(N-ethylaminocarbonyl)phenyl, 3-(aminocarbonylmethyl)phenyl, 6-phenylpyridin-3-yl, 1-(tetrahydro-2H-pyran-3-yl)-1H-pyrazol-4-yl, 1-(1-methoxypropan-2-yl)-1H-pyrazol-4-yl, 1-(2-ethoxyethyl)-1H-pyrazol-4-yl, 1-acetyl-2,5-dihydro-1H-pyrrol-3-yl, 1-acetyl-1,2,5,6-tetrahydropyridin-3-yl, 1-propionyl-1,2,5,6-tetrahydropyridin-3-yl, 1-propionyl-2,5-dihydro-1H-pyrrol-3-yl, 1-((1S,3S)-3-hydroxycyclobutyl)-1H-pyrazol-4-yl, 2,5-dihydro-1H-pyrrol-3-yl, 1,2,5,6-tetrahydropyridin-3-yl, 1-methyl-1,2,5,6-tetrahydropyridin-3-yl, 1-acryloyl-1,2,5,6-tetrahydropyridin-3-yl, 1-acryloyl-2,5-dihydro-1H-pyrrol-3-yl, 4-chloro-3,5-dimethylphenyl, 4-cyano-3-methylphenyl, 1-oxo-2,3-dihydro-1H-inden-5-yl, 3,4-bis(trifluoromethyl)phenyl, 3-methyl-4-(trifluoromethyl)phenyl, 1-(benzo[b]thiophen-7-ylmethyl)-1H-pyrazol-4-yl, 4-fluoro-3-(N-cyclohexylaminocarbonyl)phenyl, 4-morpholino-phenyl, 4-(4-(tert-butoxycarbonyl)piperazin-1-yl)phenyl, 3-chloro-5-methylphenyl, 3-(methylsulfonyl)phenyl, 4-(methylsulfonylamino)phenyl, 4-(morpholinomethyl)phenyl, 3-morpholinophenyl, 1-(2-(vinylcarbonylamino)ethyl)-1H-pyrazol-4-yl, 1-(2-aminoethyl)-1H-pyrazol-4-yl, 3-cyclopropyl-4-methyl phenyl, 3-ethoxyphenyl, 3-(hydroxymethyl)phenyl, 1-(2-(tert-butoxycarbonylamino)ethyl)-1H-pyrazol-4-yl, 3-phenethoxyphenyl, 1,2,3,6-tetrahydropyridin-4-yl, 1-(2-(vinylsulfonylamino)ethyl)-1H-pyrazol-4-yl, 4-(phenylamino)-phenyl, 3-methyl-1H-pyrazol-4-yl, 4-(benzyloxy)phenyl, 3,5-difluorophenyl, 3-fluoro-5-trifluoromethylphenyl, 3-(ethylsulfonyl)phenyl, 3-(trifluoromethoxy)phenyl, 1-(thiazol-5-ylmethyl)-1H-pyrazol-4-yl, p-tolyl, 4-cyclopropylphenyl, 4-(ethylsulfonyl)phenyl, 1-(6-vinylpyridin-2-yl)methyl)-1H-pyrazol-4-yl, 6-(benzyloxy)pyridin-3-yl, 1-(tert-butoxy-carbonyl)-2,5-dihydro-1H-pyrrol-3-yl, 1-(2-hydroxy-1-phenylethyl)-1H-pyrazol-4-yl, 1-(2-cyano-1-phenylethyl)-1H-pyrazol-4-yl, 6-cyclopropylpyridin-3-yl, 4-cyano-3-methoxy-phenyl, 4-methoxy-3-(trifluoromethyl)phenyl, 4-chlorophenyl, 1-(3,4-difluorobenzyl)-1H-pyrazol-4-yl, 4-methyl-3-(trifluoromethyl)phenyl, 4-(pyrrolidine-1-carbonyl)phenyl, 4-(isopropylaminocarbonyl)phenyl, 4-(4-methylpiperazin-1-yl)phenyl, 3-chloro-5-cyano-phenyl, 3-(pyrrolidine-1-carbonyl)phenyl, 3-(methylsulfonylaminomethyl)phenyl, 3-(1H-pyrazol-5-yl)phenyl, 4-(methylsulfonyl)phenyl, 4-(cyclopropylaminocarbonyl)phenyl, 1-(2-fluoroethyl)-1H-pyrazol-4-yl, 3-(cyclopropylmethoxy)phenyl, 3-(benzyloxy)phenyl, 3-(morpholinomethyl)phenyl, 3-(phenoxymethyl)phenyl, 1-(3-fluorophenyl)-1H-pyrazol-4-yl, 2-cyclopropylvinyl, 6-(trifluoromethyl)pyridin-3-yl, 1-(4-fluorophenyl)-1H-pyrazol-4-yl, 2,4-dimethylthiazol-5-yl, 1-propyl-1H-pyrazol-4-yl, 1-butyl-1H-pyrazol-4-yl, 1-(2-(phenylamino)ethyl)-1H-pyrazol-4-yl, 4-(aminocarbonyl)phenyl, 4-(N-methylamino-carbonyl)phenyl, 3-fluoro-4-(N-methylaminocarbonyl)phenyl, 1-(2-(3,3-difluoroazetidin-1-yl)ethyl)-1H-pyrazol-4-yl, 1-(2-(3,3-difluoropyrrolidin-1-yl)ethyl)-1H-pyrazol-4-yl, 1-(2-((2,2,2-trifluoroethyl)amino)ethyl)-1H-pyrazol-4-yl, 1-propenyl, 3-(methylcarbonyl-amino)phenyl, 4-(methylsulfonylamino)phenyl, 4-(morpholine-4-carbonyl)phenyl, 4-(4-acetylpiperazin-1-yl)phenyl, 1-(2,2-difluoroethyl)-1H-pyrazol-4-yl, 5-isopropylfuran-2-yl, 1-(3,3-difluorocyclopentyl)-1H-pyrazol-4-yl, 141S,3R)-3-hydroxycyclopentyl)-1H-pyrazol-4-yl, 1-((1S,3S)-3-hydroxycyclopentyl)-1H-pyrazol-4-yl , 3-(1H-pyrazol-4-yl)phenyl, 5-bromofuran-2-yl, 3-(phenylamino)phenyl, 2-methylthiazol-5-yl, 3-(phenylethynyl)phenyl, 3-phenethylphenyl, 1-(3-fluorocyclopentyl)-1H-pyrazol-4-yl, 1-(1-methoxy-2-methyl-propan-2-yl)-1H-pyrazol-4-yl, 1-(1-acryloylazetidin-3-yl)-1H-pyrazol-4-yl, 1-(1-propionyl-azetidin-3-yl)-1H-pyrazol-4-yl, 6-oxo-1,6-dihydropyridin-3-yl, 4-(piperazin-1-yl)phenyl, 1-(1-fluoro-2-methylpropan-2-yl)-1H-pyrazol-4-yl, 3-(trifluoromethyl)-1H-pyrazol-4-yl, 3,5-dimethylphenyl, 4-(morpholinosulfonyl)phenyl, 3-(4-methylpiperazine-1-carbonyl)phenyl, 3-(2-hydroxypropan-2-yl)phenyl, 1-isopropyl-3-methyl-1H-pyrazol-4-yl, 1-isopropyl-5-methyl-1H-pyrazol-4-yl, 3-cyclopropyl-1H-pyrazol-5-yl, 5-methoxycarbonylpyrrol-3-yl, 3-cyclopropyl-1-isopropyl-1H-pyrazol-5-yl, 5-cyclopropyl-1-isopropyl-1H-pyrazol-3-yl, 1-isopropyl-5-(methoxycarbonyl)pyrrol-3-yl, 1-methyl-3-(trifluoromethyl)-1H-pyrazol-5-yl, 1-isopropyl-1H-pyrazol-3-yl, 1-cyclopentyl-5-cyclopropyl-1H-pyrazol-3-yl, 1-cyclopentyl-3-cyclopropyl-1H-pyrazol-5-yl, 1-cyclopentyl-1H-pyrazol-3-yl, 1-isopropyl-1H-pyrazol-5-yl, 1-isopropyl-5-(N-methylaminocarbonyl)pyrrol-3-yl, 1-isopropyl-5-(N,N-dimethylamino-carbonyl)pyrrol-3-yl, 1-(2-cyclopropylethyl)-1H-pyrazol-3-yl, 1-(2-cyclopropylethyl)-1H-pyrazol-5-yl, 1-ethyl-1H-pyrazol-3-yl, 3-(3,3-dimethyl-2-oxopyrrolidin-1-yl)phenyl, 3-(2-oxo-3-phenylpyrrolidin-1-yl)phenyl, 3-((E)-styryl)phenyl, 3-(3-cyanophenyl)phenyl, 3-(3-(methylsulfonylamino)phenyl)phenyl, 3-(4-(methylsulfonylamino)phenyl)phenyl, or 3-(4-(N-methylaminosulfonyl)phenyl)phenyl.

In certain embodiments, R3 is 1H-pyrazol-4-yl, 1-(cyclopropylmethyl)-1H-pyrazol-4-yl, 1-(1-methylcyclopropyl)-1H-pyrazol-4-yl, 5-fluoro-1H-pyrazol-4-yl, 1-(2-phenylpropan-2-yl)-1H-pyrazol-4-yl, 1-(pyridin-3-yl)-1H-pyrazol-4-yl, 1-(pyridin-4-yl)-1H-pyrazol-4-yl, 1-(pyridin-2-yl)-1H-pyrazol-4-yl, 1-[1-(N-methylaminocarbonyl)-1,1-dimethylmethyl]-1H-pyrazol-4-yl, 5-fluoro-1-isopropyl-1H-pyrazol-4-yl, 1-(cyclopropylmethyl)-1H-pyrazol-5-yl, 1-(cyclopropylmethyl)-1H-pyrazol-3-yl, 1-(tetrahydro-2 H-thiopyran-4-yl)-1H-pyrazol-4-yl, 1-(1,1-dioxidotetrahydro-2H-thiopyran-4-yl)-1H-pyrazol-4-yl, 1-((6-(3-oxobut-1-en-1-yl)pyridin-2-yl)methyl)-1H-pyrazol-4-yl, 3-iodophenyl, 3-methyl-1,2,4-oxadiazol-5-yl, 5-methyl-1,3,4-oxadiazol-2-yl, 1H-imidazol-2-yl, 5-phenyloxazol-2-yl, 1-cyclohexylpyrazol-4-yl, 1-isopropylpyrazol-4-yl, biphenyl-3-yl, 3-((4-fluorophenyl)amino)phenyl, 3-(2-oxopyrrolidin-1-yl)phenyl, 3-(methylcarbonylamino)-5-phenylphenyl, 3-furyl, benzofuran-3-yl, 1-phenyl-1H-pyrazol-3-yl, 5-cyclopropylfuran-2-yl, 2-methylfuran-3-yl, 1-phenyl-1H-pyrazol-4-yl, 1-ethyl-1H-pyrazol-4-yl, 1-methyl-6-oxo-1,6-dihydropyridin-3-yl, furan-2-yl, 5-phenylfuran-2-yl, 1-isopropyl-1H-pyrazol-4-yl, pyrimidin-5-yl, 5-methylpyridin-3-yl, 1-methyl-1H-pyrazol-3-yl, 4-phenylfuran-2-yl, 2-fluorophenyl, 4-cyanophenyl, 4-methoxyphenyl, 4-(trifluoromethyl)-phenyl, 4-fluorophenyl, 1-benzyl-1H-pyrazol-4-yl, 5-chloropyridin-3-yl, 5-fluoropyridin-3-yl, 1-methyl-1H-pyrazol-5-yl, 4-(hydroxymethyl)furan-2-yl, 3-cyanophenyl, 2,5-dihydrofuran-3-yl, thiophen-3-yl, thiophen-2-yl, 1-methyl-1H-pyrazol-4-yl, 5-methylfuran-2-yl, 5-(hydroxymethyl)furan-2-yl, 3-(trifluoromethyl)-phenyl, 3-methoxyphenyl, 3-fluorophenyl, pyridin-3-yl, 1-(methylsulfonyl)-1H-pyrazol-4-yl, 1-cyclopentyl-1H-pyrazol-4-yl, 1-(thiophen-3-ylmethyl)-1H-pyrazol-4-yl, 4-chloro-3-(morpholine-4-carbonyl)phenyl, 3-chloro-4-(cyclopropylaminocarbonyl)phenyl, 1-(1-hydroxy-2-methylpropan-2-yl)-1H-pyrazol-4-yl, 1-(3-methoxybenzyl)-1H-pyrazol-4-yl, 1-(pyridin-4-ylmethyl)-1H-pyrazol-4-yl, 1-(2-chlorobenzyl)-1H-pyrazol-4-yl, 1-(3-phenoxy-benzyl)-1H-pyrazol-4-yl, 1-(4-phenoxybenzyl)-1H-pyrazol-4-yl, 1-cyclohexyl-1H-pyrazol-4-yl, 1-(1-phenylethyl)-1H-pyrazol-4-yl, 1-cyclobutyl-1H-pyrazol-4-yl, 1-(sec-butyl)-1H-pyrazol-4-yl, 4-fluoro-3-(pyrrolidine-1-carbonyl)phenyl, 1-(cyclopropylsulfonyl)-1H-pyrazol-3-yl, 1-(cyclopropanecarbonyl)-1H-pyrazol-3-yl, 1-(2-cyclopropylethyl)-1H-pyrazol-4-yl, 1-([1,1′-biphenyl]-3-ylmethyl)-1H-pyrazol-4-yl, 1-phenethyl-1H-pyrazol-4-yl, 1-(2-methoxybenzyl)-1H-pyrazol-4-yl, 1-(4-methoxybenzyl)-1H-pyrazol-4-yl, 1-(tert-butyl)-1H-pyrazol-4-yl, 3,4-dimethylphenyl, 3-chloro-4-ethoxyphenyl, 4-methoxy-3-methylphenyl, 2-methylbenzo[d]thiazol-5-yl, 1-(2-phenoxybenzyl)-1H-pyrazol-4-yl, 1-(phenylsulfonyl)-1H-pyrazol-4-yl, 1-benzoyl-1H-pyrazol-4-yl, 1-benzhydryl-1H-pyrazol-4-yl, 1-([1, 1′-biphenyl]-2-ylmethyl)-1H-pyrazol-4-yl, 1-(cyclohexylmethyl)-1H-pyrazol-4-yl, 1-(pyridin-3-ylmethyl)-1H-pyrazol-4-yl, benzofuran-2-yl, 5-ethylfuran-2-yl, 1-(2-methoxyethyl)-1H-pyrazol-4-yl, 1-(naphthalen-1-ylmethyl)-1H-pyrazol-4-yl, 1-([1,1′-biphenyl]-4-ylmethyl)-1H-pyrazol-4-yl, 3-phenoxyphenyl, 3,4-dichlorophenyl, 3-chloro-4-methoxyphenyl, 3-methoxy-4-methylphenyl, 1-(thiazol-4-ylmethyl)-1H-pyrazol-4-yl, 1H-indazol-5-yl, 3,4-dimethoxyphenyl, 4-methoxy-3, 5-dimethylphenyl, 1-(oxetan-3-yl)-1H-pyrazol-4-yl, 1-(2-fluorobenzyl)-1H-pyrazol-4-yl, 1-(4-fluorobenzyl)-1H-pyrazol-4-yl, 1-(methoxycarbonylmethyl)-1H-pyrazol-4-yl, 1-(2-(dimethylamino)ethyl)-1H-pyrazol-4-yl, 3-cyano-4-methylphenyl, benzo[d][1,3]dioxol-5-yl, 2,3-dihydrobenzofuran-5-yl, 1-(3-fluorobenzyl)-1H-pyrazol-4-yl, 1-(thiophen-2-ylmethyl)-1H-pyrazol-4-yl, 1-(2,2,2-trifluoroethyl)-1H-pyrazol-4-yl, 1-(3-chlorobenzyl)-1H-pyrazol-4-yl, 1-isobutyl-1H-pyrazol-4-yl, 1-(3,3,3-trifluoropropyl)-1H-pyrazol-4-yl, 1-(difluoromethyl)-1H-pyrazol-4-yl, 1-(2-cyanoethyl)-1H-pyrazol-4-yl, 4-cyclopropylfuran-2-yl, 2,2-difluorobenzo[d][1,3]dioxol-5-yl, 3-fluoro-4-(aminocarbonyl)phenyl, 3-fluoro-4-(methylsulfonyl)phenyl, 3-chloro-4-(trifluoromethoxy)phenyl, 5-fluoro-3-(aminocarbonyl)phenyl, 3-(hydroxymethyl)-4-methoxyphenyl, 1-(methylsulfonyl)-1H-pyrrol-3-yl, 1-methyl-1H-pyrrol-3-yl, 3-bromophenyl, 3-(1-methylpyrazol-4-yl)phenyl, 3-(1-isopropylpyrazol-4-yl)phenyl, 4-phenylphenyl, 4-(4-fluoroanilino)phenyl, 3-(tert-butoxycarbonylamino)phenyl, 1-acetyl-1,2,3,6-tetrahydropyridin-4-yl, 1-propionyl-1,2,3,6-tetrahydropyridin-4-yl, 1-acryloyl-1,2,3,6-tetrahydropyridin-4-yl, 1-methyl-1,2,3,6-tetrahydropyridin-4-yl, 1-((2-methylthiazol-4-yl)methyl)-1H-pyrazol-4-yl, 1-(2-(acetylamino)ethyl)-1H-pyrazol-4-yl, 3,5-dichlorophenyl, 2-fluoro-4-(methylsulfonyl)phenyl, 1-(tert-pentyl)-1H-pyrazol-4-yl, 3-(2-morpholinoethyl)phenyl, 3-(2-(dimethylamino)ethyl)phenyl, 1-(1-(thiazol-4-yl)ethyl)-1H-pyrazol-4-yl, 1-(tetrahydro-2H-pyran-4-yl)-1H-pyrazol-4-yl, 3-methoxy-4-(trifluoro-methyl)phenyl, 3-methoxycarbonyl-4-chlorophenyl, 4-(trifluoromethoxy)phenyl, 3-methyl-4-(trifluoromethoxy)phenyl, 4-cyclopropyl-3-(trifluoromethyl)phenyl, 2,2-dimethyl-2,3-dihydrobenzofuran-5-yl, 3,5-dimethoxyphenyl, 3,4-difluorophenyl, 4-biphenyl, 3-chloro-5-fluorophenyl, 3,5-bis(trifluoromethyl)phenyl, 3-fluoro-5-methoxyphenyl, 3-(amino-carbonyl)phenyl, 4-(cyclopropylmethoxy)phenyl, 2-fluoro-5-(benzyloxycarbonyl)phenyl, 3-(1H-pyrazol-1-yl)phenyl, 1-(2-hydroxycyclopentyl)-1H-pyrazol-4-yl, 3-(N-methylamino-sulfonyl)phenyl, 4-(2-hydroxypropan-2-yl)phenyl, 2-(trifluoromethyl)pyridin-4-yl, 6-phenoxypyridin-3-yl, 2-methoxypyridin-4-yl, 4-methyl-2-phenylthiazol-5-yl, 3-amino-5-cyanophenyl, 1-(tetrahydrofuran-3-yl, 3-(N-ethylaminocarbonyl)phenyl, 3-(aminocarbonylmethyl)phenyl, 6-phenylpyridin-3-yl, 1-(tetrahydro-2H-pyran-3-yl)-1H-pyrazol-4-yl, 1-(1-methoxypropan-2-yl)-1H-pyrazol-4-yl, 1-(2-ethoxyethyl)-1H-pyrazol-4-yl, 1-acetyl-2,5-dihydro-1H-pyrrol-3-yl, 1-acetyl-1,2,5,6-tetrahydropyridin-3-yl, 1-propionyl-1,2,5,6-tetrahydropyridin-3-yl, 1-propionyl-2,5-dihydro-1H-pyrrol-3-yl, 1-((1S,3S)-3-hydroxycyclobutyl)-1H-pyrazol-4-yl, 2,5-dihydro-1H-pyrrol-3-yl, 1,2,5,6-tetrahydropyridin-3-yl, 1-methyl-1,2,5,6-tetrahydropyridin-3-yl, 1-acryloyl-1,2,5,6-tetrahydropyridin-3-yl, 1-acryloyl-2,5-dihydro-1H-pyrrol-3-yl, 4-chloro-3,5-dimethylphenyl, 4-cyano-3-methylphenyl, 1-oxo-2,3-dihydro-1H-inden-5-yl, 3,4-bis(trifluoromethyl)phenyl, 3-methyl-4-(trifluoromethyl)phenyl, 1-(benzo[b]thiophen-7-ylmethyl)-1H-pyrazol-4-yl, 4-fluoro-3-(N-cyclohexylaminocarbonyl)phenyl, 4-morpholinophenyl, 4-(4-(tert-butoxy-carbonyl)piperazin-1-yl)phenyl, 3-chloro-5-methylphenyl, 3-(methylsulfonyl)phenyl, 4-(methylsulfonylamino)-phenyl, 4-(morpholinomethyl)phenyl, 3-morpholinophenyl, 1-(2-(vinylcarbonylamino)ethyl)-1H-pyrazol-4-yl, 1-(2-aminoethyl)-1H-pyrazol-4-yl, 3-cyclopropyl-4-methylphenyl, 3-ethoxyphenyl, 3-(hydroxymethyl)phenyl, 1-(2-(tert-butoxy-carbonylamino)ethyl)-1H-pyrazol-4-yl, 3-phenethoxyphenyl, 1,2,3,6-tetrahydropyridin-4-yl, 1-(2-(vinylsulfonylamino)ethyl)-1H-pyrazol-4-yl, 4-(phenylamino)phenyl, 3-methyl-1H-pyrazol-4-yl, 4-(benzyloxy)phenyl, 3,5-difluorophenyl, 3-fluoro-5-trifluoromethylphenyl, 3-(ethylsulfonyl)phenyl, 3-(trifluoromethoxy)-phenyl, 1-(thiazol-5-ylmethyl)-1H-pyrazol-4-yl, p-tolyl, 4-cyclopropylphenyl, 4-(ethylsulfonyl)-phenyl, 1-(6-vinylpyridin-2-yl)methyl)-1H-pyrazol-4-yl, 6-(benzyloxy)pyridin-3-yl, 1-(tert-butoxycarbonyl)-2,5-dihydro-1H-pyrrol-3-yl, 1-(2-hydroxy-1-phenylethyl)-1H-pyrazol-4-yl, 1-(2-cyano-1-phenylethyl)-1H-pyrazol-4-yl, 6-cyclopropylpyridin-3-yl, 4-cyano-3-methoxyphenyl, 4-methoxy-3-(trifluoromethyl)-phenyl, 4-chlorophenyl, 1-(3,4-difluorobenzyl)-1H-pyrazol-4-yl, 4-methyl-3-(trifluoromethyl)phenyl, 4-(pyrrolidine-1-carbonyl)phenyl, 4-(isopropylamino-carbonyl)-phenyl, 4-(4-methylpiperazin-1-yl)phenyl, 3-chloro-5-cyanophenyl, 3-(pyrrolidine-1-carbonyl)phenyl, 3-(methylsulfonylaminomethyl)phenyl, 3-(1H-pyrazol-5-yl)phenyl, 4-(methylsulfonyl)phenyl, 4-(cyclopropylaminocarbonyl)phenyl, 1-(2-fluoroethyl)-1H-pyrazol-4-yl, 3-(cyclopropylmethoxy)phenyl, 3-(benzyloxy)phenyl, 3-(morpholino-methyl)phenyl, 3-(phenoxymethyl)phenyl, 1-(3-fluorophenyl)-1H-pyrazol-4-yl, 2-cyclopropylvinyl, 6-(trifluoromethyl)pyridin-3-yl, 1-(4-fluorophenyl)-1H-pyrazol-4-yl, 2,4-dimethylthiazol-5-yl, 1-propyl-1H-pyrazol-4-yl, 1-butyl-1H-pyrazol-4-yl, 1-(2-(phenylamino)ethyl)-1H-pyrazol-4-yl, 4-(aminocarbonyl)phenyl, 4-(N-methylamino-carbonyl)phenyl, 3-fluoro-4-(N-methylamino-carbonyl)phenyl, 1-(2-(3,3-difluoroazetidin-1-yl)ethyl)-1H-pyrazol-4-yl, 1-(2-(3,3-difluoropyrrolidin-1-yl)ethyl)-1H-pyrazol-4-yl, 1-(2-(2,2,2-trifluoroethyl)amino)ethyl)-1H-pyrazol-4-yl, 1-propenyl, 3-(methylcarbonyl-amino)phenyl, 4-(methylsulfonylamino)phenyl, 4-(morpholine-4-carbonyl)phenyl, 4-(4-acetylpiperazin-1-yl)phenyl, 1-(2,2-difluoroethyl)-1H-pyrazol-4-yl, 5-isopropylfuran-2-yl, 1-(3,3-difluorocyclopentyl)-1H-pyrazol-4-yl, 141S,3R)-3-hydroxycyclopentyl)-1H-pyrazol-4-yl, 1-((1S,3S)-3-hydroxycyclopentyl)-1H-pyrazol-4-yl, 3-(1H-pyrazol-4-yl)phenyl, 5-bromofuran-2-yl, 3-(phenylamino)phenyl, 2-methylthiazol-5-yl, 3-(phenylethynyl)phenyl, 3-phenethylphenyl, 1-(3-fluorocyclopentyl)-1H-pyrazol-4-yl, 1-(1-methoxy-2-methyl-propan-2-yl)-1H-pyrazol-4-yl, 1-(1-acryloylazetidin-3-yl)-1H-pyrazol-4-yl, 1-(1-propionyl-azetidin-3-yl)-1H-pyrazol-4-yl, 6-oxo-1,6-dihydropyridin-3-yl, 4-(piperazin-1-yl)phenyl, 1-(1-fluoro-2-methylpropan-2-yl)-1H-pyrazol-4-yl, 3-(trifluoromethyl)-1H-pyrazol-4-yl, 3,5-dimethylphenyl, 4-(morpholinosulfonyl)phenyl, 3-(4-methylpiperazine-1-carbonyl)phenyl, 3-(2-hydroxypropan-2-yl)phenyl, 1-isopropyl-3-methyl-1H-pyrazol-4-yl, 1-isopropyl-5-methyl-1H-pyrazol-4-yl, 3-cyclopropyl-1H-pyrazol-5-yl, 5-methoxycarbonylpyrrol-3-yl, 3-cyclopropyl-1-isopropyl-1H-pyrazol-5-yl, 5-cyclopropyl-1-isopropyl-1H-pyrazol-3-yl, 1-isopropyl-5-(methoxycarbonyl)pyrrol-3-yl, 1-methyl-3-(trifluoromethyl)-1H-pyrazol-5-yl, 1-isopropyl-1H-pyrazol-3-yl, 1-cyclopentyl-5-cyclopropyl-1H-pyrazol-3-yl, 1-cyclopentyl-3-cyclopropyl-1H-pyrazol-5-yl, 1-cyclopentyl-1H-pyrazol-3-yl, 1-isopropyl-1H-pyrazol-5-yl, 1-isopropyl-5-(N-methylaminocarbonyl)pyrrol-3-yl, 1-isopropyl-5-(N, N-dimethylamino-carbonyl)-pyrrol-3-yl, 1-(2-cyclopropylethyl)-1H-pyrazol-3-yl, 1-(2-cyclopropylethyl)-1H-pyrazol-5-yl, 1-ethyl-1H-pyrazol-3-yl, 3-(3,3-dimethyl-2-oxopyrrolidin-1-yl)phenyl, 3-(2-oxo-3-phenylpyrrolidin-1-yl)phenyl, 3-((E)-styryl)phenyl, 3-(3-cyanophenyl)phenyl, 3-(3-(methylsulfonylamino)phenyl)phenyl, 3-(4-(methylsulfonylamino)phenyl)phenyl, or 3-(4-(N-methylaminosulfonyl)phenyl)phenyl.

In certain embodiments, R3 is aryl or heteroaryl, wherein each aryl and heteroaryl is optionally substituted with one or more groups Rx; provided R3 is not phenyl, fluorophenyl, chlorophenyl, pyridyl, nitrophenyl, or propylisoxazole.

In certain embodiments, R3 is pyrazol-4-yl, optionally substituted with Rx.

In certain embodiments, Rx is C1-6 alkyl, that is substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, and —N(Rv)-S(O)2-Rv.

In certain embodiments, Rx is C1-6alkyl that is optionally substituted with Rxa.

In certain embodiments, R3 is pyrazol-4-yl, substituted with Rx.

In certain embodiments, R3 is phenyl that is substituted with oxo, C1-6 alkyl, C2-6 alkenyl, C2-6alkynyl, carbocyclyl, aryl, heteroaryl, heterocycle, —N(Rv)2, —CN, —C(O)—N(Rv)2, —S(O)—N(Rv)2, —S(O)2-N(Rv)2, —O—Rv, —S—Rv, —O—C(O)—Rv, —O—C(O)—O—Rv, —C(O)—Rv, —C(O)—O—Rv, —S(O)—Rv, —S(O)2-Rv, —O—C(O)—N(Rv)2, —N(Rv)-C(O)—ORv, —N(Rv)-C(O)—N(Rv)2, —S(O)2-N(Rv)2, —N(Rv)-C(O)—Rv, —N(Rv)-S(O)—Rv, —N(Rv)-S(O)2-Rv, —N(Rv)-S(O)—N(Rv)2, or —N(Rv)-S(O)2-N(Rv)2, wherein any C1-6 alkyl, C2-6 alkenyl, C2-6 alkynyl, carbocyclyl, aryl, heteroaryl, and heterocycle is optionally substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, —N(Rv)-S(O)2-Rv, or C1-6alkyl that is optionally substituted with one or more groups independently selected from oxo and halo.

In certain embodiments, Rx is C2-6 alkenyl or C2-6 alkynyl, wherein any C2-6 alkenyl and C2-6alkynyl is optionally substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, and —N(Rv)-S(O)2-Rv

In certain embodiments, Rx is selected from C2-6 alkenyl, C2-6 alkynyl, carbocyclyl, aryl, heteroaryl, heterocycle, —F, —Cl, —Br, —I, —NO2, —N(Rv)2, —CN, —C(O)—N(Rv)2, —S(O)—N(Rv)2, —S(O)2-N(Rv)2, —O—Rv, —S—Rv, —O—C(O)—Rv, —O—C(O)—O—Rv, —C(O)—Rv, —C(O)—O—Rv, —S(O)—Rv, —S(O)2-Rv, —O—C(O)—N(Rv)2, —N(Rv)-C(O)—ORv, —N(Rv)-C(O)—N(Rv)2, —S(O)2-N(Rv)2, —N(Rv)-C(O)—Rv, —N(Rv)-S(O)—Rv, —N(Rv)-S(O)2-Rv, —N(Rv)-S(O)—N(Rv)2, and —N(Rv)-S(O)2-N(Rv)2, wherein any C2-6 alkenyl, C2-6 alkynyl, carbocyclyl, aryl, heteroaryl, and heterocycle is optionally substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, —N(Rv)-S(O)2-Rv, and C1-6 alkyl that is optionally substituted with one or more groups independently selected from oxo and halo.

In certain embodiments, R3 is heteroaryl that is substituted with oxo, C1-6 alkyl, C2-6 alkenyl, C2-6 alkynyl, carbocyclyl, aryl, heteroaryl, heterocycle, —F, —Cl, —Br, —I, —NO2, —N(Rv)2, —CN, —C(O)—N(Rv)2, —S(O)—N(Rv)2, —S(O)2-N(Rv)2, —O—Rv, —S—Rv, —O—C(O)—Rv, —O—C(O)—O—Rv, —C(O)—Rv, —C(O)—O—Rv, —S(O)—Rv, —S(O)2-Rv, —O—C(O)—N(Rv)2, —N(Rv)-C(O)—ORv, —N(Rv)-C(O)—N(Rv)2, —S(O)2-N(Rv)2, —N(Rv)-C(O)—Rv, —N(Rv)-S(O)—Rv, —N(Rv)-S(O)2-Rv, —N(Rv)-S(O)—N(Rv)2, or —N(Rv)-S(O)2-N(Rv)2; wherein any C1-6 alkyl is substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, and —N(Rv)-S(O)2-Rv; and wherein any C2-6alkenyl, C2-6alkynyl, carbocyclyl, aryl, heteroaryl, and heterocycle is optionally substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, —N(Rv)-S(O)2-Rv, and C1-6 alkyl that is optionally substituted with one or more groups independently selected from oxo and halo.

In certain embodiments, R3 is a 5-membered heteroaryl that is substituted with oxo, C1-6 alkyl, C2-6 alkenyl, C2-6 alkynyl, carbocyclyl, aryl, heteroaryl, heterocycle, —F, —Cl, —Br, —I, —NO2, —N(Rv)2, —CN, —C(O)—N(Rv)2, —S(O)—N(Rv)2, —S(O)2-N(Rv)2, —O—Rv, —S—Rv, —O—C(O)—Rv, —O—C(O)—O—Rv, —C(O)—Rv, —C(O)—O—Rv, —S(O)—Rv, —S(O)2-Rv, —O—C(O)—N(Rv)2, —N(Rv)-C(O)—ORv, —N(Rv)-C(O)—N(Rv)2, —S(O)2-N(Rv)2, —N(Rv)-C(O)—Rv, —N(Rv)-S(O)—Rv, —N(Rv)-S(O)2-Rv, —N(Rv)-S(O)—N(Rv)2, or —N(Rv)-S(O)2-N(Rv)2; wherein any C1-6alkyl, is substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, and —N(Rv)-S(O)2-Rv; and wherein any C2-6alkenyl, C2-6alkynyl, carbocyclyl, aryl, heteroaryl, and heterocycle is optionally substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, —N(Rv)-S(O)2-Rv, and C1-6 alkyl that is optionally substituted with one or more groups independently selected from oxo and halo.

In certain embodiments, R3 is phenyl that is substituted with oxo, C1-6 alkyl, C2-6 alkenyl, C2-6 alkynyl, carbocyclyl, aryl, heteroaryl, heterocycle, —N(Rv)2, —CN, —C(O)—N(Rv)2, —S(O)—N(Rv)2, —S(O)2-N(Rv)2, —O—Rv, —S—Rv, —O—C(O)—Rv, —O—C(O)—O—Rv, —C(O)—Rv, —C(O)—O—Rv, —S(O)—Rv, —S(O)2-Rv, —O—C(O)—N(Rv)2, —N(Rv)-C(O)—ORv, —N(Rv)-C(O)—N(Rv)2, —S(O)2-N(Rv)2, —N(Rv)-C(O)—Rv, —N(Rv)-S(O)—Rv, —N(Rv)-S(O)2-Rv, —N(Rv)-S(O)—N(Rv)2, or —N(Rv)-S(O)2-N(Rv)2; wherein any C1-6 alkyl, is substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, and —N(Rv)-S(O)2-Rv; and wherein any C2-6 alkenyl, C2-6 alkynyl, carbocyclyl, aryl, heteroaryl, and heterocycle is optionally substituted with one or more groups independently selected from Rxa, oxo, halo, —NO2, —N(Rv)2, —CN, —C(O)—N(Rv)2, —S(O)—N(Rv)2, —S(O)2-N(Rv)2, —O—Rv, —S—Rv, —O—C(O)—Rv, —C(O)—Rv, —C(O)—O—Rv, —S(O)—Rv, —S(O)2-Rv, —C(O)—N(Rv)2, —S(O)2-N(Rv)2, —N(Rv)-C(O)—Rv, —N(Rv)-C(O)—ORv, —N(Rv)-S(O)—Rv, —N(Rv)-S(O)2-Rv, and C1-6alkyl that is optionally substituted with one or more groups independently selected from oxo and halo.

In certain embodiments, R2 and R3 taken together with the atoms to which they are attached form a cyclohexyl ring, which is optionally substituted with one or more groups Rx.

In certain embodiments, R2 and R3 taken together with the atoms to which they are attached form a phenyl ring, which is optionally substituted with one or more groups Rx.

In certain embodiments, R4 is H, methyl, ethyl, propyl, cyclopropylmethyl, 2-hydroxyethyl, 2-(dimethylamino)ethyl, phenyl, benzyl, or 2-methoxyethyl.

In certain embodiments, R4 and R3 taken together with the atoms to which they are attached form a heterocyclyl.

In certain embodiments, the compound is other than any one of the following:

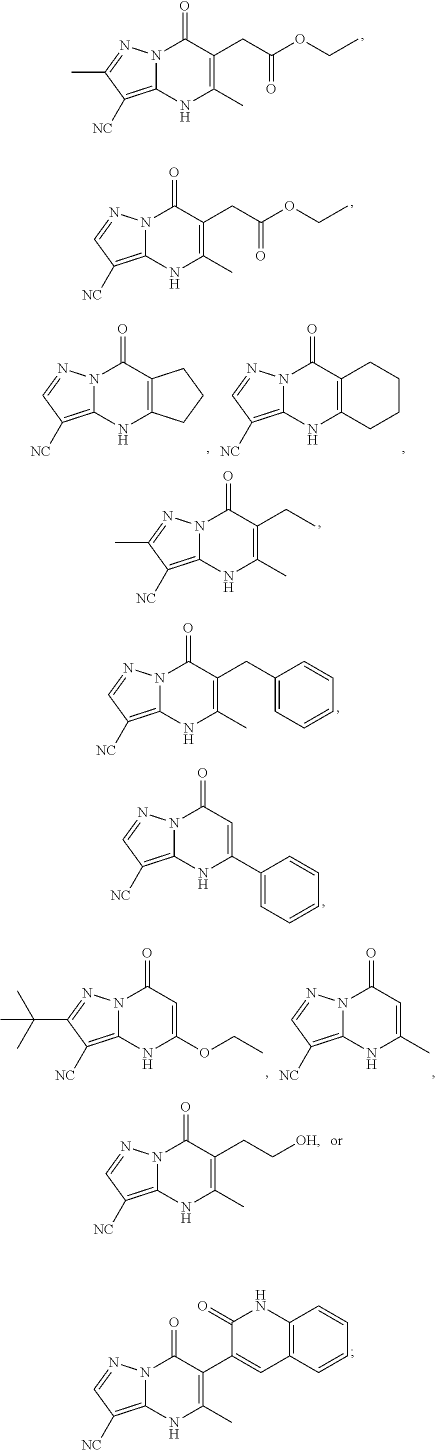

In other embodiments, the agent is a (small molecule) inhibitor of jumonji at-rich interactive domain 1a (jarid1a) and 1b (jarid1b) histone demethylase, such as described in PCT publication WO2014055634A1 (incorporated by reference herein).

In another embodiment, the JMJD3 demethylase inhibitor comprises any of the N-2-(2-pyridinyl)-4-pyrimidinyl-beta-alanine derivatives as disclosed in WO2012052390 to Atkinson et al., which is hereby incorporated by reference in its entirety. Exemplary inhibitors include those having the structure of:

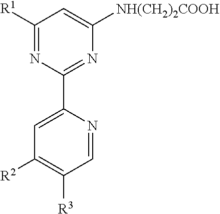

where R1 is C1-6 alkyl; C3-7 cycloalkyl; C1-6 haloalkyl; a 5, 6 or 7-membered aryl or heteroaryl (which heteroaryl contains one or more heteroatoms selected from N, O and S and which is optionally fused to phenyl), said 5, 6 or 7-membered aryl or heteroaryl being optionally substituted with one or more substituents independently selected from C1-3alkyl; O—C1-6alkyl (which is optionally substituted by phenyl or naphthyl, each of which may be substituted by one of more substituents independently selected from halo); —O-cyclohexyl (which is optionally fused with phenyl); C(O)NRc 2; or NRaRb, each Ra and Rb is independently selected from: H; C1-3alkyl which is optionally substituted by one or more substituents independently selected from phenyl (which phenyl is optionally substituted by one or more substituents independently selected from C1-3alkyl, O—C1-3alkyl, C(O)NRc 2, halo and cyano), C(O)NRc 2, a 4, 5, 6 or 7-membered heterocyclic or heteroaryl group (containing one or more heteroatoms independently selected from N, O and, S), a 3, 4, 5, 6 or 7-membered cycloalkyl group (which is optionally fused to phenyl), halo, OC1-3alkyl, OH, —NHCOC1-3alkyl NRc 2 and C(O)NHCH₂C(O)NRc 2; a 3, 4, 5, 6 or 7-membered cycloalkyl group (which is optionally fused to phenyl), or Ra and Rb together form a 5, 6 or 7-membered heterocyclic group optionally containing one or more further heteroatoms independently selected from N, O, S or S(O)2 said heterocyclic group being optionally fused to a 5, 6 or 7-membered aryl or heteroaryl ring containing one or more heteroatoms independently selected from N, O and S; the heterocylic ring and/or the aryl or heteroaryl to which it is optionally fused being optionally substituted by one or more substituents independently selected from halo, OH, C1-3alkyl, O—C1-3alkyl, C(O)C1-3alkyl, S(O)2C1-3alkyl, NHC(O)C1-3alkyl, NHS(O)2C1-3alkyl, C(O)NRc 2, C(O)NRd 2 (wherein Rd and Rd together form a 5 or 6-membered heterocylic ring), NRC 2C(O)phenyl, S(O)2NRc 2, =0(oxo) and 5, 6 or 7-membered aryl or heteroaryl (containing one or more heteroatoms independently selected from N, O and S); R2 and R3 are each independently selected from: H, (CH2)1-3NRc(CH2)1-3NRc 2, (CH2)1-6NRc 2; C1-3 alkyl; O—C1-3alkyl; C1-3haloalkyl; (CH2)0-3NRaRb (wherein Ra and Rb are as defined above); (CH2)0-3NHPh; (CH2)0-3OPh; (CH2)0-3Ph; or R2 and R3 together form a fused phenyl ring, and each Rc is independently selected from hydrogen and C1-3alkyl or a pharmaceutically acceptable salt thereof. Suitable inhibitors of this structure include, without limitation, N-[6-(1,1-dimethylethyl)-2-(2-pyridinyl)-4-pyrimidinyl]-(3-alanine; N-[2-(2-pyridinyl)-6-(trifluoromethyl)-4-pyrimidinyl]-(3-alanine; N-[6-(4-morpholinyl)-2-(2-pyridinyl)-4-pyrimidinyl]-(3-alanine; N[6-(methylamino)-2-(2-pyridinyl)-4-pyrimidinyl]-(3-alanine; N-[2-(2-pyridinyl)-6-(1-pyrrolidinyl)-4-pyrimidinyl]-(3-alanine; N-[6-[(2-hydroxyethyl)amino]-2-(2-pyridinyl)-4-pyrimidinyl]-(3-alanine; N-[6-[(phenylmethyl)amino]-2-(2-pyridinyl)-4-pyrimidinyl]-(3-alanine; N-[6-[(2-hydroxyethyl)(methyl)amino]-2-(2-pyridinyl)-4-pyrimidinyl]-(3-alanine; N-[6-(dimethylamino)-2-(2-pyridinyl)-4-pyrimidinyl]-(3-alanine; and N-[2-(2-pyridinyl)-6-(1,2,4,5-tetrahydro-3/-/-3-benzazepin-3-yl)-4-pyrimidinyl]-(3-alanine. Other JMJD3 demethylase inhibitors disclosed in WO2012052390 to Atkinson et al., which is hereby incorporated by reference in its entirety, are also suitable for use in the methods as described herein.

In another embodiment, the JMJD3 demethylase inhibitor comprises any one of the small molecule JMJD3 inhibitors disclosed in WO2013143597 to Barker et al., which is hereby incorporated by reference in its entirety. Exemplary inhibitors include those having the structure of:

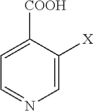

where X is —(R1)o-i-(R2)o-i-R3 or —R1-R4; Each R1 is independently NH, N(CH3), O; R2 is a linker group with a maximum length of 5 atoms between R1 and R3 and is selected from: —CO—C1-6 alkyl-, —CO—, —CO—C1-6 alkyl-O—, —CO—C1-6 alkyl-S—, —CO—C1-6 alkyl-O—C1-6 alkyl-, —C1-6 alkyl-, C1-6alkyl-O—, —C1-6alkyl-SO2-, —C1-6alkyl-NH—CO—, —C1-3 alkyl-C3-6 cycloalkyl-C1-3alkyl-O— wherein each alkyl is straight chain or branched and may be optionally substituted by one or more substituents independently selected from phenyl or —OH; R3 is selected from: a C6-12 mono or bicyclic aryl group, (each of which may be optionally substituted one or more times by substituents independently selected from halo, C1-6alkyl, C1-6 haloalkyl, C1-6alkoxy, NHCOC1-3alkyl, —O— phenyl, —CH2-phenyl, phenyl (optionally substituted by C1-3alkyl), OH, NH2, CONH2, CN, —NHCOC1-3alkylNH2, —HCOC1-3alkyl, NHCOOC1-3alkyl, —NHSO2C1-3alkyl, —SO2C1-3alkyl or —NHCOC1-3alkyl-NHCOC1-4 alkyl

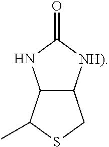

a 5-12 membered mono or bicyclic heteroaryl group (optionally substituted by one or more substituents independently selected from phenyl, CH2phenyl, —C1-6 alkyl, -oxo), a 5 or 6 membered heterocyclic group containing one or more heteromoieties independently selected from N, S, SO, SO2 or O and optionally fused to a phenyl group (optionally substituted by one or more substituents independently selected from phenyl, CH2phenyl, C1-6 alkyl), or a 3-7 membered cycloalkyl (including bridged cycloalkyl) and optionally fused to a phenyl group (and optionally substituted by one or more substituents independently selected from OH, phenyl, CH2 phenyl), R4 is selected from: C1-6 straight chain or branched alkyl (optionally substituted by NH2), COC1-6 straight chain or branched alkyl. Suitable inhibitors of this structure include, without limitation, 3-{[(4-chlorophenyl)acetyl]amino}-4-pyridinecarboxylic acid; 3-{[(4-methylphenyl)acetyl]amino}-4-pyridinecarboxylic acid; -[(3-phenylpropanoyl)amino]-4-pyridinecarboxylic acid; -[(phenylcarbonyl)amino]-4-pyridinecarboxylic acid; -[(2,2-dimethylpropanoyl)amino]-4-pyridinecarboxylic acid; -{[(phenyloxy)acetyl]amino}-4-pyridinecarboxylic acid; -{[4-(4-methylphenyl)butanoyl]amino}-4-pyridinecarboxylic acid; -[(2-naphthalenylacetyl)amino]-4-pyridinecarboxylic acid; -{[4-(2-naphthalenyl)butanoyl]amino}-4-pyridinecarboxylic acid; and -{[4-(4-bromophenyl)butanoyl]amino}-4-pyridinecarboxylic acid. Additional small molecule JMJD3 inhibitors disclosed in WO2013143597 to Barker et al., which is hereby incorporated by reference in its entirety, are also suitable for use in the methods described herein.

Small molecule JMJD3 demethylase inhibitors can be readily modified using techniques known in the art to increase bioavailability (see Hetal et al, “A Review on Techniques for Oral Bioavailability Enhancement of Drugs,” Intl. J. Pharm. Sci. Rev. Res. 4(3): 203-223 (2010) and Huttunen et al., “Prodrugs—from Serendipity to Rational Design,” Pharmacol. Rev. 63(3):750-771 (2011), which are hereby incorporated by reference in their entirety). For example, common modifications to increase the solubility and dissolution rate of small molecules include particle size reduction, modification of the crystal habit, dispersion in carriers, inclusion complexation, salt formation, and change in pH. Modification of the small molecule into a prodrug form using, for example, attached ionizable or polar neutral groups (e.g., phosphate esters, amino acids, sugar moieties) is also known to enhance solubility and dissolution rate. Common modification to increase permeability and absorption include, for example, conversion of hydrophilic hydroxyl, thiol, carboxyl, phosphate, or amine groups to more lipophilic alkyl or aryl esters.

In another embodiment, the JMJD3 demethylase inhibitor is a JMJD3 antisense RNA, shRNA, or siRNA oligonucleotide.

The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,179,796 to Cowsert et al., which are hereby incorporated by reference in their entirety). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an mRNA molecule (see e.g., Weintraub, H. M., “Antisense DNA and RNA,” Scientific Am. 262:40-46 (1990), which is hereby incorporated by reference in its entirety). The antisense nucleic acid molecule hybridizes to its corresponding target nucleic acid molecule, such as JMJD3, to form a double-stranded molecule, which interferes with translation of the mRNA, as the cell will not translate a double-stranded mRNA. Antisense nucleic acids used in the methods of the present disclosure are typically at least 10-12 nucleotides in length, for example, at least 15, 20, 25, 50, 75, or 100 nucleotides in length. The antisense nucleic acid can also be as long as the target nucleic acid with which it is intended to form an inhibitory duplex. Antisense nucleic acids can be introduced into cells as antisense oligonucleotides, or can be produced in a cell in which a nucleic acid encoding the antisense nucleic acid has been introduced, for example, using gene therapy methods.

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the JMJD3 nucleotide sequence (SEQ ID NO: 1). siRNA molecules are typically designed to target a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy, have been described and are suitable for use in accordance with this aspect of the disclosure (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway. shRNA molecules that effectively interfere with JMJD3 expression have been developed, as described herein, and have the following nucleic acid sequences:

(SEQ ID NO: 1)
5′-CAGGGAAGTTTCGAGAAGTCCTATAGTGAAGCC
CAAGATGTATAGGACTCTCGAAC TTCCCTT-3′
and
(SEQ ID NO: 2)
5′-ACACCAGCAGTAGCAACAGCAATAGTGAAGCCA
CAGATGTATTGCTGTTGCTACTG CTGGTGG-3′

b. EGFR/HER2 InhibitorsHistone Demethylase Inhibitors

In certain embodiments, the anti-PESC agent is a receptor tyrosine kinase inhibitor, and is preferably an EGFR inhibitor, a HER2 inhibitor or a dual EGFR/HER2 inhibitor.

Exemplary EGFR inhibitors/antagonists include, inter alia, small-molecule EGFR inhibitors/antagonists, such as gefitinib, erlotinib, lapatinib, afatinib (also referred to as BIBW2992), neratinib, ABT-414, dacomitinib (also referred to as PF-00299804), AV-412, PD 153035, vandetanib, PKI-166, pelitinib (also referred to as EKB-569), canertinib (also referred to as CI-1033), icotinib, poziotinib (also referred to as NOV120101), BMS-690514, CUDC-101, AP26113 or XL647.

In certain embodiments, the anti-PESC agent is an EGFR tyrosine kinase inhibitor (EGFR-TKI). Exemplary EGFR-TKI include afatinib, erlotinib, gefitinib, neratinib, dacomitinib and osimertinib.

WZ8040 is a novel mutant-selective irreversible EGFRT790M inhibitor, does not inhibit ERBB2 phosphorylation (T798I).

c. Proteasome Inhibitors

In other embodiments, the anti-PESC agent can be a proteasome inhibitor.

The proteasome inhibitor may be any proteasome inhibitor known in the art. In particular, it is one of the proteasome inhibitors described in more detail in the following paragraphs.

Suitable proteasome inhibitors for use in combinations described herein include (a) peptide boronates, such as bortezomib (also known as Velcade™ and PS341), delanzomib (also known as CEP-18770), ixazomib(also known as MLN9708) or ixazomib citrate; (b) peptide aldehydes, such as MG132 (Z-Leu-Leu-Leu-H), MG115 (Z-Leu-Leu-Nva-H), IPSI 001, fellutamide B, ALLN (Ac-Leu-Leu-N1e-H, also referred to as calpain inhibitor I), and leupeptin (Ac-Leu-Leu-Arg-al); (c) peptide vinyl sulfones, (d) epoxyketones, such as epoxomicin, oprozomib (also referred to as PR-047 or ONX 0912), PR-957 (also known as ONX 0914), and carfilzomib (also referred to as PR-171); and (e) [3-lactones, such as lactacystin, omuralide, salinosporamide A (also known as NPI-0052 and marizomib), salinosporamide B, belactosines, cinnabaram ides, polyphenols, TMC-95, and PS-519.

In a preferred embodiment, the proteasome inhibitor is bortezomib, also known as VELCADE and PS341. In a preferred embodiment, the proteasome inhibitor is [(1R)-3-methyl-1-[[(2S)-3-phenyl-2-(pyrazine-2-carbonylamino)propanoyl]amino]butyl]boronic acid, or a pharmaceutically acceptable salt, solvate, hydrate, cocrystal, or prodrug thereof. In a preferred embodiment, the proteasome inhibitor is the compound of Formula (44):

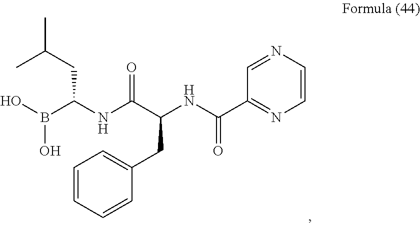 [SMILES: CC(C)C[C@H](NC(=O)[C@H](Cc1ccccc1)NC(=O)c1cnccn1)B(O)O]

or a pharmaceutically acceptable salt, solvate, hydrate, cocrystal, or prodrug thereof. Bortezomib is commercially available.

In a preferred embodiment, the proteasome inhibitor is carfilzomib, also known as PR-171 or KYPROLIS. In a preferred embodiment, the proteasome inhibitor is (2S)-4-methyl-N-[(2S)-1-[[(2S)-4-methyl-1-[(2R)-2-methyloxiran-2-yl]-1-oxopentan-2-yl]amino]-1-oxo-3-phenylpropan-2-yl]-2-[[(2S)-2-[(2-morpholin-4-ylacetyl)amino]-4-phenylbutanoyl]amino]pentanamide, or a pharmaceutically acceptable salt, solvate, hydrate, cocrystal, or prodrug thereof. In a preferred embodiment, the proteasome inhibitor is the compound of Formula (45):

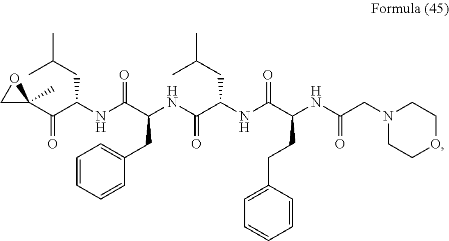 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

or a pharmaceutically acceptable salt, solvate, hydrate, cocrystal, or prodrug thereof. Carfilzomib is commercially available.

In a preferred embodiment, the proteasome inhibitor is delanzomib, also known as CEP-18770. In a preferred embodiment, the proteasome inhibitor is [(1R)-1-[[(2S,3R)-3-hydroxy-2-[(6-phenylpyridine-2-carbonyl)amino]butanoyl]amino]-3-methylbutyl]boronic acid, or a pharmaceutically acceptable salt, solvate, hydrate, cocrystal, or prodrug thereof. In a preferred embodiment, the proteasome inhibitor is the compound of Formula (46):

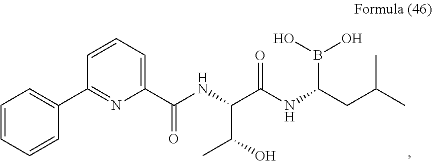 [SMILES: CC(C)C[C@H](NC(=O)[C@@H](NC(=O)c1cccc(-c2ccccc2)n1)[C@@H](C)O)B(O)O]

or a pharmaceutically acceptable salt, solvate, hydrate, cocrystal, or prodrug thereof.

In a preferred embodiment, the proteasome inhibitor is ixazomib, also known as MLN-9708 or ixazomib citrate. In a preferred embodiment, the proteasome inhibitor is 4-(carboxymethyl)-24(R)-1-(2-(2,5-dichlorobenzamido)acetamido)-3-methylbutyl)-6-oxo-1,3,2-dioxaborinane-4-carboxylic acid, or a pharmaceutically acceptable salt, solvate, hydrate, cocrystal, or prodrug thereof. In a preferred embodiment, the proteasome inhibitor is 1,3,2-dioxaborolane-4,4-diacetic acid, 2-[(1R)-1-[[2-[(2,5-dichlorobenzoyl)amino]acetyl]amino]-3-methylbutyl]-5-oxo-, or a pharmaceutically acceptable salt, solvate, hydrate, cocrystal, or prodrug thereof. In a preferred embodiment, the proteasome inhibitor is 2,2′-{2-[(1R)-1-{[N-(2,5-dichlorobenzoyl)glycyl]amino}-3-methylbutyl]-5-oxo-1,3,2-dioxaborolane-4,4-diyl}diacetic acid, or a pharmaceutically acceptable salt, solvate, hydrate, cocrystal, or prodrug thereof. In a preferred embodiment, the proteasome inhibitor is the compound of Formula (47):

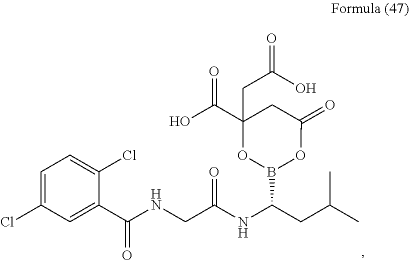 [SMILES: CC(C)C[C@H](NC(=O)CNC(=O)c1cc(Cl)ccc1Cl)B1OC(=O)CC(CC(=O)O)(C(=O)O)O1]

or a pharmaceutically acceptable salt, solvate, hydrate, cocrystal, or prodrug thereof. In a preferred embodiment, the proteasome inhibitor is 1B-{(1R)-1-[2-(2,5-dichlorobenzamido)acetamido]-3-methylbutyl}boronic acid, or a pharmaceutically acceptable salt, solvate, hydrate, cocrystal, or prodrug thereof. In a preferred embodiment, the proteasome inhibitor is the compound of Formula (48):

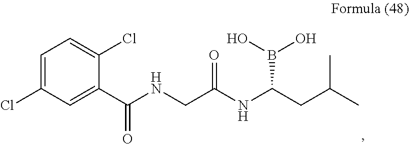 [SMILES: CC(C)C[C@H](NC(=O)CNC(=O)c1cc(Cl)ccc1Cl)B(O)O]

or a pharmaceutically acceptable salt, solvate, hydrate, cocrystal, or prodrug thereof. Ixazomib citrate is commercially available.

In a preferred embodiment, the proteasome inhibitor is marizomib, also known as NPI-0052 and Salinosporamide A. In a preferred embodiment, the proteasome inhibitor is (4R,5S)-4-(2-chloroethyl)-1-((1S)-cyclohex-2-enyl(hydroxy)methyl)-5-methyl-6-oxa-2-azabicyclo[3.2.0]heptane-3,7-dione, or a pharmaceutically acceptable salt, solvate, hydrate, cocrystal, or prodrug thereof. In a preferred embodiment, the proteasome inhibitor is the compound of Formula (49):

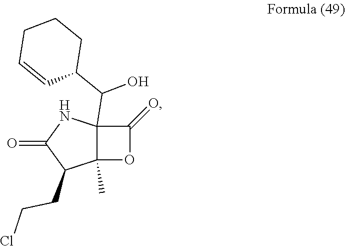 [SMILES: C[C@@]12OC(=O)C1(C(O)[C@@H]1C=CCCC1)NC(=O)[C@@H]2CCCl]

or a pharmaceutically acceptable salt, solvate, hydrate, cocrystal, or prodrug thereof.

In a preferred embodiment, the proteasome inhibitor is oprozimib, also known as PR-047 or ONX 0912. In a preferred embodiment, the proteasome inhibitor is N-[(2S)-3-methoxy-1-[[(2S)-3-methoxy-1-[[(2S)-1-[(2R)-2-methyloxiran-2-yl]-1-oxo-3-phenylpropan-2-yl]amino]-1-oxopropan-2-yl]amino]-1-oxopropan-2-yl]-2-methyl-1,3-thiazole-5-carboxamide, or a pharmaceutically acceptable salt, solvate, hydrate, cocrystal, or prodrug thereof. In a preferred embodiment, the proteasome inhibitor is the compound of Formula (50):

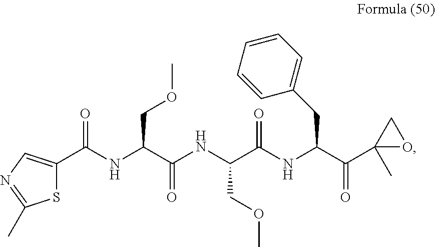 [SMILES: COC[C@H](NC(=O)c1cnc(C)s1)C(=O)N[C@@H](COC)C(=O)N[C@@H](Cc1ccccc1)C(=O)C1(C)CO1]

or a pharmaceutically acceptable salt, solvate, hydrate, cocrystal, or prodrug thereof.

Exemplary proteasome inhibitors include bortezomib, carfilzomib, ixazomib, oprozomib or marizomib

CEP-18770, disulfiram, epigallocatechin-3-gallate, epoxomicin, lactacystin, MG132, MLN9708, ONX 0912, and salinosporamide A.

In other embodiments, the anti-PESC agent is Delanzomib (CEP-18770), an orally active inhibitor of the chymotrypsin-like activity of proteasome with IC₅₀ of 3.8 nM, with only marginal inhibition of the tryptic and peptidylglutamyl activities of the proteosome.

In other embodiments, the anti-PESC agent is CEP-18770, an novel orally-active inhibitor of the chymotrypsin-like activity of the proteasome with a cellular IC₅₀ value of 3.8 nM.

d. STAT3 Inhibitors

In certain embodiments, the anti-PESC agent is a STAT3 inhibitor such as Stattic. Stattic is nonpeptidic small molecule that potently inhibits STAT3 activation and nuclear translocation with IC₅₀ of 5.1 μM in cell-free assays, highly selectivity over STAT1.

Non-limiting examples of STAT3 inhibitors include BP-1-102, S3I-M2001, STA-21, S3I-201, Galiellalactone, a polypeptide having the sequence PY*LKTK (where Y* represents phosphotyrosine), and a polypeptide having the sequence Y*LPQTV (where Y* represents phosphotyrosine). Additional non-limiting examples of STAT3 inhibitors are described in Yue and Turkson Expert Opin Investig Drugs. 2009 January; 18(1): 45-56, the entire content of which is incorporated herein by reference.

Other STAT3 inhibitors include: E1: 4_-Bromo-phenyl -2-N-aminoacyl-1 1-dioxide-benzo [b] thiophene; E2: 4_-bromo-2-N-(4-fluorophenyl) alanyl-1,1-dioxide, benzo [b] thiophene; E3: 4_-bromo-benzo 2-N-(4-methoxyphenyl) alanyl-1,1-dioxide [b] thiophene; E4: 4_-bromo-2-N-aminoacyl-p-tolyl-1,1-oxidation benzo [b] thiophene; E5: 4_-bromo-2-N-(4-chlorophenyl) alanyl-1,1-dioxide, benzo [b] thiophene; E6: 4_-bromo-2-N-benzo (3-chlorophenyl) alanyl-1,1-dioxide [b] thiophene; E7: 4_-bromo-2-N-(2-chlorophenyl) alanyl-1,1-dioxide benzo [b] thiophene; E8: 4_-bromo-2-N-(3-chloro-4-fluorophenyl) alanyl-1,1-dioxide, benzo [b] thiophene; E9: 4_-chloro-2-N-aminoacyl-phenyl-1,1-dioxide, benzo [b] thiophene; E10: 5_-bromo-phenyl-2-N-aminoacyl-1,1-dioxide, benzo [b] thiophene; EII: 6_bromo-phenyl-2-N-aminoacyl-1,1-dioxide, benzo [b] thiophene; E12: 2-N-aminoacyl-phenyl-1,1-dioxide, benzo [b] thiophene; E13: 5_-nitro-phenyl-2-N-Acyl-1,1-dioxide, benzo [b] thiophene; E14: 5_-bromo-n-butyl-2-N-aminoacyl-1,1-dioxide, benzo [b] thiophene; E15: 5_bromo-2-N-aminoacyl-t-butyl-1,1-dioxide, benzo [b] thiophene; E16: 5_-bromo-2-N-isopropyl-alanyl-1,1-benzo [b] dioxide thiophene; E17: 5_-bromo-2-N-cyclohexyl-alanyl-1,1-benzo [b] thiophene dioxide; E18: 5_-bromo-2-N-[(3s, 5s, 7s)-I-adamantyl]-1,1-aminoacyl dioxide benzo [b] thiophene; E19: 4_-bromo-benzo-2-N-benzyl-aminoacyl-1,1-dioxide [b] thiophene; E20: 4_-bromo-2-N-(4-bromophenethyl) benzo-1,1-dioxide aminoacyl [b] thiophene; E21: 5_-bromo-2-N-(4-phenoxy-phenyl) amino-benzo-1,1-dioxide group [b] thiophene; E22: 5_-bromo-2-N-[4-(I-piperidinyl-carbonyl) phenyl]-1,1-aminoacyl dioxide benzo [b] thiophene; E23: 5_-bromo-2-N-[4-(4-morpholin-ylcarbonyl) phenyl] carboxamido-1,1-dioxide, benzo [b] thiophene; E24: 5_-bromo-2-N-[4-(N-methyl-N-phenyl) carbamoyl] phenyl-carboxamido-1,1-dioxide, benzo [b] thiophene; E25: 4_bromo-2-p-tolyl-carboxy-1,1-dioxide [b] thiophene; E26: 5_bromo-2-N, N-diethyl-1,1-dioxide aminoacyl benzothienyl; E27: 5_-bromo-2-(I-pyrrolyl) carbonyl-1,1-dioxide, benzo [b] thiophene; E28: 5_-bromo-2-(I-piperidyl) carbonyl-1,1-dioxide, benzo [b] thiophene; E29: 5_-bromo-2-(2-methyl-piperidine yl) carbonyl-1,1-dioxide, benzo [b] thiophene; E30: benzo 5_-bromo-2-(3-methyl-1-piperidinyl) carbonyl-1,1-dioxide [b] thiophene; E31: 5_-bromo-2-morpholino-carbonyl-1,1-dioxide, benzo [b] thiophene; E32: 5_-bromo-2-(4-ethyl-1-piperazinyl) carbonyl-1,1-dioxide-benzo [b] thiophene; E33: 5_-bromo-2-(N-methyl-N-phenyl) benzo-1,1-dioxide aminoacyl [b] thiophene; E34: 4 _bromo-2-(I-piperidinyl) carbonyl-1,1-dioxide, benzo [b] thiophene; E35: 5_trifluoromethyl-2-(I-piperidinyl) carbonyl-1,1-dioxide benzo [b] thiophene; E36: 4_-bromo-2-methoxycarbonyl-1,1-dioxide, benzo [b] thiophene; E37: 2_methoxycarbonyl-1,1-oxidation benzo [b] thiophene; E38: benzo 5_acetamido-2-N-phenyl-1,1-dioxide aminoacyl [b] thiophene; E39: 5_benzoylamino-2-N-aminoacyl phenyl-1,1-dioxide, benzo [b] thiophene; E40: 5_of Methylbenzamido-2-N-aminoacyl-phenyl-1,1-dioxide, benzo [b] thiophene; E41: 5_Trifluoromethyl-benzoyl-phenylcarbamoyl group-2-N-acyl-1,1-dioxide, benzo [b] thiophene; E42: 5_p-chlorobenzoyl-N-phenylcarbamoyl group an acyl -2-1,1-benzo [b] thiophene dioxide; E43: 5_-cyclohexyl-carboxamido-2-N-phenyl-aminoacyl-1,1-dioxide, benzo [b] thiophene; or E44: 5_benzamido-2-(I-piperidinyl) carbonyl 1,1-dioxide, benzo [b] thiophene.

e. GSK3 Inhibitors

In certain embodiments, the anti-PESC agent is an inhibitor of glycogen synthase kinase 3 (GSK-3), also referred to here as a GSK-3 inhibitor. In certain embodiments, the GSK-3 inhibitor can be an inhibitor of both GSK-3α and GSK-3β. In certain embodiments, the GSK-3 inhibitor is a selective inhibitor of GSK-3α relative to GSK-3β. In certain embodiments, the GSK-3 inhibitor is a selective inhibitor of GSK-3β relative to GSK-3α.

GSK3 inhibitor such as AZD1080. AZD1080 is a selective, orally active, brain permeable GSK3 inhibitor, inhibits human GSK3α and GSK3β with Ki of 6.9 nM and 31 nM, respectively, shows >14-fold selectivity against CDK2, CDK5, CDK1 and Erk2.

Other examples of inhibitors of GSK-3 include aloisines (such as aloisine A and aloisine B), hymenialdisine (such as dibromohymenialdisine), indirubins (such as 5,5′-dibromo-indirubin), maleimides, in particular macrocyclic bisindolylmaleimides (such as Ro 31-8220, SB-216763, SB-415286, or 3F8), and muscarinic agonists (such as AFI02B and AFI50).

In certain embodiments, the GSK3 inhibitor can be 6-bromoindirubin-3′-oxime (BIO), CHIR-99021, SB216763, CHIR-98014, TWS119, IM-12, 1-Azakenpaullone, AR-A014418, SB415286, AZD1080, AZD2858, indirubin, A 1070722, TCS 2002, Tideglusib, or any derivatives thereof.

In another embodiment, the GSK-3 inhibitor comprises flavopiridol, kenpaullone, alsterpaullone, azakenpaullone, pyrazolopyridine, CHIR98014, CHIR99021, CHIR-637, CT20026, SU9516, ARA014418, and staurosporine.

Exemplary GSK3 inhibitor can be selected from the group consisting of Li+, (2′Z,3′E)-6-bromoindirubin-3′-oxime (BIO), (2Z,3′EJ-6-bromoindirubin-3′-acetoxime (BIO-acet-oxime), SB-216763 (3-(2,4-dichlorophenyl)-4-(1-methyl-1H-indol-3-yl)-1H-pyrrole-2,5-dione), SB-415286 (3-[(3-chloro-4-hydroxyphenyl)amino]-4-(2-nitrophenyl)-1H-pyrrol-2,5-dione), enzastaurin (3-(1-methylindol-3-yl)-4-[1-[1-(pyridin-2-ylmethyl)piperidin-4-yl]indol-3-yl]pyrrole-2,5-dione), L803-mts (Myr-N-Gly-Lys-Glu-Ala-Pro-Pro-Ala-Pro-Pro-Gln-pSer(P0₃H)-Pro-NH₂), NP031 1 12 (4-benzyl-2-naphtalen-1-yl-1,2,4-thiadiazolidine-3,5-dione), paliperidone palmitate (3-(2-(4-(6-fluoro-1,2-benzisoxazol-3-yl)-1-piperidinyl)ethyl)-6,7,8,9-tetrahydro-2-methyl-4-oxo-4H-pyrido(1,2-a)pyrimidin-9-yl ester), valproic acid (2-propylpentanoic acid), TDZD-8 (4-benzyl-2-methyl-1,2,4-thiadiazolidine-3,5-dione), and 9-hydroxyrisperidone (6,7,8,9-tetrahydro-3-(2-(4-(6-fluoro-1,2-benzisoxazol-3-yl)-1-piperidinyl)ethyl)-9-hydroxy-2-methyl-4H-pyrido[2,1-a]pyrimidin-4-one).

Among GSK-3 inhibitors, derivatives of the bis-indole indirubin (collectively referred to as indirubins) appear as a class of original and promising tools and agents. Their moderate selectivity might be inconvenient when used as a research reagent, but their combined effects on several disease-relevant targets (in particular CDKs and GSK-3) may constitute an advantage for potential therapeutic applications. Among many indirubins, 6-bromo-indirubin-3′-oxime (BIO) has been widely used to investigate the physiological role of GSK-3 in various cellular settings and to alter the fate of embryonic stem cells.

In one embodiment, the GSK-3 inhibitor is a compound of formula:

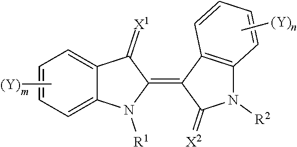

wherein X¹ and X², independently of each other, are O, S, N—OR³, N(Z¹), or two groups independently selected from H, F, Cl, Br, I, N0₂, phenyl, and (CrC₆)alkyl, and wherein R³ is hydrogen, (C C₆)alkyl, or (C C₆)alkyl-C(0)-;

each Y, independently of each other, is H, (C C₆)alkyl, (C C₆)alkyl-C(0)-, (C C₆)alkyl-C(0)0-, phenyl, N(Z¹)(Z²), sulfonyl, phosphonyl, F, Cl, Br, or I;

Z¹ and Z², independently of each other, are H, (d-C₆)alkyl, phenyl, benzyl, or Z¹ and Z² together with the nitrogen to which they are attached represent a 5, 6, or 7-membered heterocyclyl;

n and m, independently of each other, are 0, 1 , 2, 3, or 4;

R¹ and R², independently of each other, are H, (CrC₆)alkyl, (CrC₆)alkyl-C(0)-, phenyl, benzyl, or benzoyl;

and wherein alkyi is branched or straight-chain alkyi, optionally substituted with 1, 2, 3, 4, or 5 OH, N(Z¹)(Z²), (CrC₆)alkyl, phenyl, benzyl, F, Cl, Br, or I; and wherein any phenyl, benzyl, or benzoyl is optionally substituted with 1, 2, 3, 4, or 5 OH, N(Z¹)(Z²), (C C₆)alkyl, F, Cl, Br, or I;

or a salt thereof.

In one embodiment, X¹ is O and X² is N—OH, or X¹ is N—OH and X² is O. In another embodiment, one Y is Br. In another embodiment, one Y is Br at the 6′-position. In another embodiment, n is 0 and m is 1 , or n is 1 and m is 0. In another embodiment R¹ and R² are

H.

In one embodiment, the GSK-3 inhibitor comprises 6-bromoindirubin-3′-oxime of formula

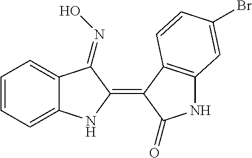 [SMILES: O=C1Nc2cc(Br)ccc2/C1=C1/Nc2ccccc2/C1=N\O]

or a salt thereof.

In one embodiment, the GSK-3 inhibitor comprises 6-bromoindirubin-3′-acetoxime of formula

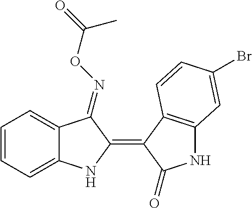 [SMILES: CC(=O)O/N=C1/C(=C2/C(=O)Nc3cc(Br)ccc32)Nc2ccccc21]

or a salt thereof.

f. HSP90, HSP70 and Dual HSP90/70 Inhibitors

In certain embodiments, the anti-PESC agent is an HSP90 Inhibitor or an HSP70 inhibitor or both. An exemplary anti-PESC agent is Nanchangmycin

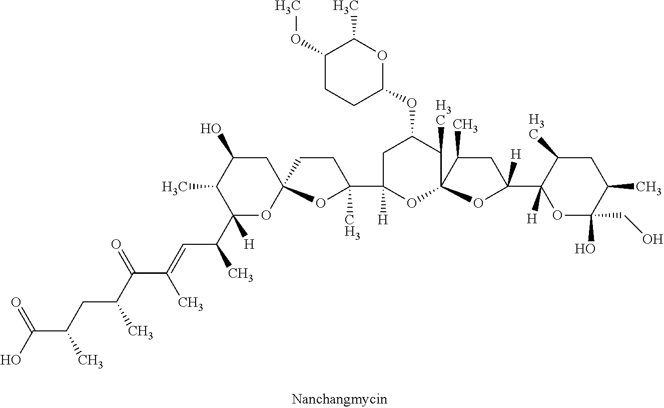 [SMILES: CO[C@H]1CC[C@@H](O[C@H]2C[C@H]([C@]3(C)CC[C@]4(C[C@H](O)[C@@H](C)[C@@H]([C@@H](C)/C=C(\C)C(=O)[C@H](C)C[C@H](C)C(=O)O)O4)O3)O[C@]3(O[C@@H]([C@H]4O[C@@](O)(CO)[C@H](C)C[C@@H]4C)C[C@@H]3C)[C@@H]2C)O[C@H]1C]

Other examples of Hsp90 inhibitors include, but are not limited to, geldanamycin, radicicol, 17-N-Allylamino-17-demethoxygeldanamycin/tanespicmycin/17AAG (BMS), herbimycin A, novobiocin sodium (U-6591), 17-GMB-APA-GA, macbecin I, CCT 018159, gedunin, PU24FC1, PU-H71, PU-DZ8, PU3, AUY922 (Novartis), HSP990 (Novartis), retaspimycin hydrochloride/IPI-504 (Infinity), BIIB021/CNF2024 (Biogen Idec), STA-9090 (Synta), SNX-5422/mesylate (Pfizer), BIIB028 (Biogen Idec), KW-2478 (Kyowa Hakko Kirin), AT13387 (Astex), XL888 (Exelixis), MPC-3100 (Myriad), ABI-010/nab (nanoparticle, albumin bound)-17AAG (Abraxis), 17-aminodemethoxygeldanamycin (IPI-493), 17-dimethylaminoethylamino-17-demethoxygeldanamycin (17-DMAG), XL888, SNX-2112, SNX-7081, ganetespib (STA-9090), AUY922, Debio0932, BIIB028, BIIB021, MPC-3100, MPC-0767, PU3, PU-H58, DS-2248, KW-2478, CCT018159, CCT0129397, BJ-B11, elesclomol (STA-4783), G3130, gedunin, herbimycin, radester, KNK437, HSP990, or NVP-BEP800.

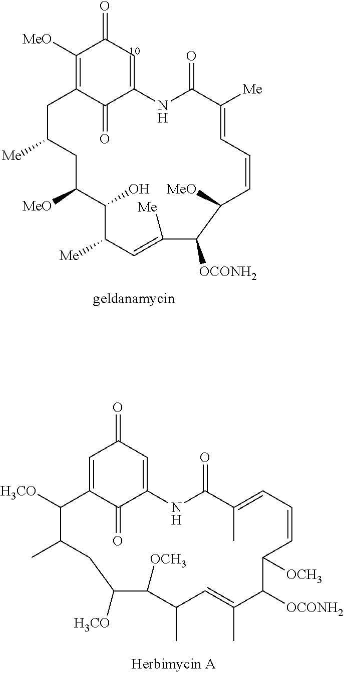

### IV. COMBINATION THERAPIES—ESO REGENERATIVE AGENT

In certain embodiments, the anti-PESC agent can be administered conjointly with one or more agents that selectively promote proliferation or other regenerative and wound healing activities of normal epithelial stem cells. Conjoint administration of these “ESO Regenerative agents” may be accomplished by administration of a single co-formulation, by simultaneous administration or by administration at separate times.

In certain embodiments, the anti-PESC agent can be administered conjointly with one or more agents that selectively promote proliferation or other regenerative and wound healing activities of normal esophageal stem cells. Conjoint administration of these “esophageal ESO Regenerative agents” may be accomplished by administration of a single co-formulation, by simultaneous administration or by administration at separate times.

a. ABL Kinase Inhibitor

In certain embodiments, the ESO Regenerative agent is pan-inhibitor of ABL kinase inhibitor, preferably a BCR-ABL kinase inhibitor. Exemplary pan-inhibitor include imatinib, nilotinib, dasatinib, bosutinib and ponatinib, and is preferably ponatinib.

b. FLT3 Inhibitors

In certain embodiments, the ESO Regenerative agent is a FLT3 inhibitor. Exemplary FLT3 inhibitors to be used herein are quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

These and further exemplary inhibitors to be used herein are described in more detail below.

Brand Name: Quizartinib

Structure:

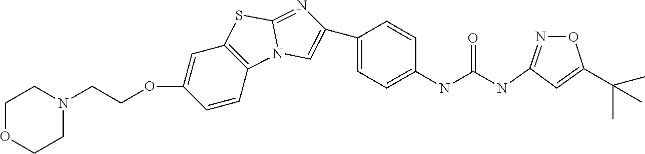 [SMILES: CC(C)(C)c1cc(NC(=O)Nc2ccc(-c3cn4c(n3)sc3cc(OCCN5CCOCC5)ccc34)cc2)no1]

Affinities: FLT3 (1.6 nM), KIT (4.8 nM), PDGFRB (7.7 nM), RET (9.9 nM), PDGFRA (11 nM), CSF1R (12 nM)

Brand Name: Crenolanib

Structure:

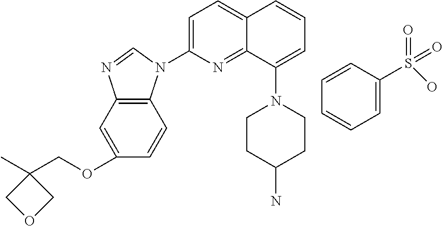

Affinities: FLT3, PDGFRb

Brand Name: Midostaurin

Structure:

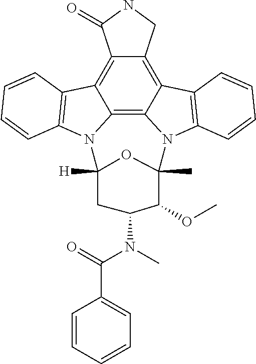 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

Affinities: PKNI (9.3 nM), TBKI (9.3 nM), FLT3 (11 nM), JAK3 (12 nM), MLKI (15 nM), and

30 targets in the range 15-110 nM

Brand Name: Lestaurtinib

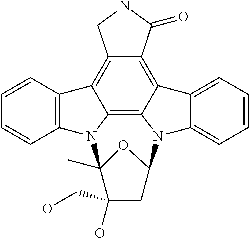

Affinities: FLT3, TRKA, TRKB, TRKC

Brand Name: 4SC-203

Structure:

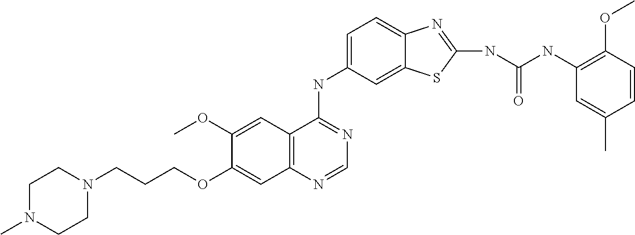

Affinities: FLT3, VEGFR

Structure:

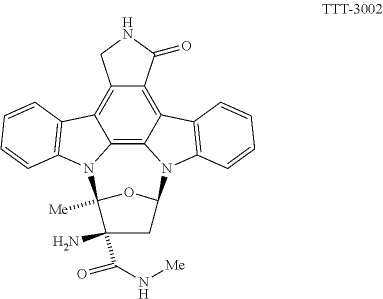 [SMILES: CNC(=O)[C@@]1(N)C[C@H]2O[C@]1(C)n1c3ccccc3c3c4c(c5c6ccccc6n2c5c31)C(=O)NC4]

Affinities: FLT3 (Wall, Blood (ASH Annual Meeting Abstracts). 2012; 120:866);

LRRK2 (Yao, Human molecular genetics. 2013; 22(2):328-44).

Clinical Phase: Preclinical

Developer: Tautatis (originator)

Brand Name: Sorafenib

Code Name: Bay-43-0006

Structure:

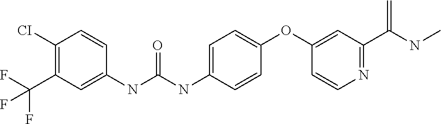

IUPAC Name: 4-[4-[3-[4-Chloro-3-(trifluoromethyl)phenyl]ureido]phenoxy]-N-methylpyridine-2-carboxamide

Affinities: DDRI (1.5 nM), HIPK4 (3 nM), ZAK (6 nM), DDR2 (7 nM), FLT3 (13 nM), and 15 targets in the range 13-130 nM (Zarrinkar, Gunawardane et al. 2009, loc. cit.) Clinical Phase: Launched (renal and heptacellular carcinoma), Phase I/O (blood cancer) Developer: Bayer

Brand Name: Ponatinib

Code Name: AP-24534 Structure:

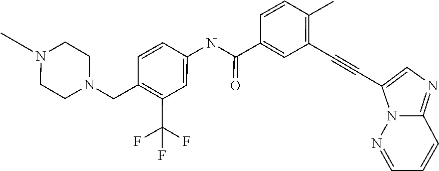 [SMILES: Cc1ccc(C(=O)Nc2ccc(CN3CCN(C)CC3)c(C(F)(F)F)c2)cc1C#Cc1cnc2cccnn12]

IUPAC Name: 3-[2-(Imidazo[I,2-b]pyridazin-3-ypethynyl]-4-methyl-N-[4-(4-methylpiperazin-I-ylmethyl)-3-(trifluoromethyl)phenyl]benzamide

Affinities: BCR-ABL, FLT3, KIT, FGFR1, PDGFRa (Gozgit, Mol Cancer Ther. 2011; 10(6):1028-35).

Clinical Phase: Phase II (AML)

Developer: Ariad Pharmaceuticals (originator)

Brand Name: Sunitinib

Code Name: SU-11248

Structure:

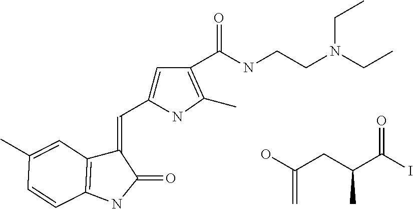

IUPAC Name: (Z)-N-[2-(Diethylamino)ethyl]-5-(5-fluoro-2-oxo-2,3-dihydro-IH-indol-3-ylidenemethyl)-2,4-dimethyl-IH-pyrrole-3-carboxamide 2(S)˜hydroxybutanedioic acid (1:1) N-[2-(Diethylamino)ethyl]-5-[(Z)-(5-fluoro-2-oxo-I,2-dihydro-3H-indol-3-ylidene)methyl]-2,4-dimethyl-1H-pyrrole-3-carboxamide L-malate

Affinities: PDGFRB (0.075 nM), KIT (0.37 nM), FLT3 (0.47 nM), PDGFRA (0.79 nM),

DRAK1 (1.0 nM), VEGFR2 (1.5 nM), FLT1 (1.8 nM), CSF1R (2.0 nM) (Zarrinkar, Gunawardane et al. 2009, loc. cit.)

Clinical Phase: Launched (renal cell carcinoma, gastrointestinal stromal cancer, neuroendocrine pancreas), phase I (AML)

Developer: Pfizer (Originator)

Brand Name: Tandutinib

Code Name: MLN-0518

Structure:

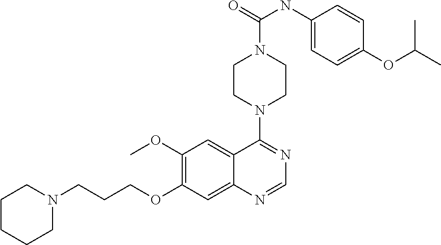 [SMILES: COc1cc2c(N3CCN(C(=O)Nc4ccc(OC(C)C)cc4)CC3)ncnc2cc1OCCCN1CCCCC1]

IUP AC Name: N-(4-Isopropoxyphenyl)-4-[6-methoxy-7-[3-(I-piperidinyl)propoxy]quinazolin-4-yl]piperazine-1-carboxamide

Affinities: PDGFRA (2.4 nM), KIT (2.7 nM), FLT3 (3 nM), PDGFRB (4.5 nM), CSF1R (4.9 nM) (Zarrinkar, Gunawardane et al. 2009, loc. cit.)

Clinical Phase: discontinued

Developer: Kyowa Hakko Kirin (Originator), Millennium Pharmaceuticals (Originator),

Code Name: FF-10101

Structure:

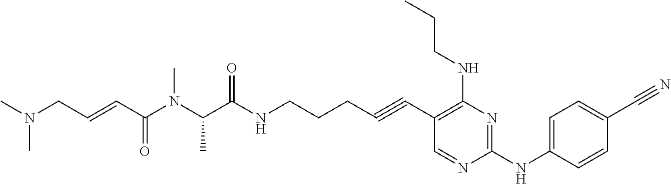 [SMILES: CCCNc1nc(Nc2ccc(C#N)cc2)ncc1C#CCCCNC(=O)[C@H](C)N(C)C(=O)/C=C/CN(C)C]

National Cancer Institute, Takeda (Originator) FLT3 inhibitors to be used in accordance with the present disclosure are not limited to the herein described or further known exemplary inhibitors. Accordingly, also further inhibitors or even yet unknown inhibitors may be used in accordance with the present disclosure. Such inliibitors may be identified by the methods described and provided herein and methods known in the art, like high-throughput screening using biochemical assays for inhibition of FLT3.

Assays for screening potential FLT3 inhibitors and, in particular, for identifying FLT3 inhibitors as defined herein, comprise, for example, in vitro competition binding assays to quantitatively measure interactions between test compounds and recombinantly expressed kinases¹ (Fabian et al; Nat Biotechnol. 2005 23(3):329-36). Hereby, competition with immobilized capture compounds and free test compounds is performed. Test compounds that bind the kinase active site will reduce the amount of kinase captured on solid support, whereas test molecules that do not bind the kinase have no effect on the amount of kinase captured on the solid support. Furthermore, inhibitor selectivity can also be assessed in parallel enzymatic assays for a set of recombinant protein kinases.^{2,3} (Davies et al., Biochem. J. 2000 35(1): 95-105; Bain et al. Biochem. J. 2003 37(1): 199-204). These assays are based on the measurement of the inhibitory effect of a kinase inhibitor and determine the concentration of compound required for 50% inhibition of the protein kinases of interest. Proteomics methods are also an efficient tool to identify cellular targets of kinase inliibitors. Kinases are enriched from cellular lysates by immobilized capture compounds, so the native target spectrum of a kinase inhibitor can be determined.⁴ (Godl et al; Proc Natl Acad Sci USA. 2003 100(26): 5434-9).

Assays for screening of potential inhibitors and, in particular, for identifying inhibitors as defined herein, are, for example, described in the following papers:
Fabian et al., Nat Biotechnol. 2005 23(3):329-36
Davies et al., Biochem. J. 2000 351 : 95-105.
Bain et al., Biochem. J. 2003 371 : 199-204.
Godl et al., Proc Natl Acad Sci USA. 2003 100(26): 15434-9.

The above papers are incorporated herein in their entirety by reference.

### V. COMBINATION THERAPIES—OTHER AGENTS

In certain embodiments, the anti-PESC agent can be administered conjointly with one or more agents that have other beneficial local activities in esophagus. Illustrative categories and specific examples of active drugs include: (a) antitussives, such as dextromethorphan, dextromethorphan hydrobromide, noscapine, carbetapentane citrate, and chlophedianol hydrochloride; (b) antihistamines, such as chlorpheniramine maleate, phenindamine tartrate, pyrilamine maleate, doxylamine succinate, and phenyltoloxamine citrate; (c) antipyretics and analgesics such as acetaminophen, aspirin and ibuprofen; (d) antacids such as aluminum hydroxide and magnesium hydroxide, (e) anti-infective agents such as antifungals, antivirals, antiseptics and antibiotics, (f) chemotherapeutic agents.

### VI. EXEMPLARY FORMULATIONS

In certain embodiments, the anti-PESC agents is formulated for topical administration as part of a bioadhesive formulation. Bioadhesive polymers have extensively been employed in transmucosal drug delivery systems and can be readily adapted for use in delivery of the subject anti-PESC agents to the esophagus, particularly the areas of lesions and tumor growth. In general terms, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups responsible for forming hydrogen bonds are the hydroxyl (—OH) and the carboxylic groups (—COOH). When these materials are incorporated into pharmaceutical formulations, drug absorption by mucosal cells may be enhanced and/or the drug may be released at the site for an extended period of time. Merely to illustrate, the bioadhesive can be a hydrophilic polymer, a hydrogel, a co-polymers/interpolymer complex or a thiolated polymer.
Hydrophilic polymers: These are water-soluble polymers that swell when they come in contact with water and eventually undergo complete dissolution. Systems coated with these polymers show high bioadhesiveness to the mucosa in dry state but the bioadhesive nature deteriorates as they start dissolving. As a result, their bioadhesiveness is short-lived. An example is poly (acrylic acid).
Hydrogels: These are three-dimensional polymer networks of hydrophilic polymers which are cross-linked either by chemical or physical bonds. These polymers swell when they come in contact with water. The extent of swelling depends upon the degree of crosslinking. Examples are polycarbophil, carbopol and polyox.
Co-polymers/Interpolymer complex: A block copolymer is formed when the reaction is carried out in a stepwise manner, leading to a structure with long sequences or blocks of one monomer alternating with long sequences of the other. There are also graft copolymers, in which entire chains of one kind (e.g., polystyrene) are made to grow out of the sides of chains of another kind (e.g., polybutadiene), resulting in a product that is less brittle and more impact-resistant. Hydrogen bonding is a major driving force for interpolymer interactions.
Thiolated polymers (Thiomers): These are hydrophilic macromolecules exhibiting free thiol groups on the polymeric backbone. Based on thiol/disulfide exchange reactions and/or a simple oxidation process disulfide bonds are formed between such polymers and cysteine-rich subdomains of mucus glycoproteins building up the mucus gel layer. So far, the cationic thiomers, chitosan-cysteine, chitosan-thiobutylamidine as well as chitosan-thioglycolic acid, and the anionic thiomers, poly (acylic acid)-cysteine, poly (acrylic acid)-cysteamine, carboxymethylcellulose-cysteine and alginate-cysteine, have been generated. Due to the immobilisation of thiol groups on mucoadhesive basis polymers, their mucoadhesive properties are 2- up to 140-fold improved.

In certain embodiments, the bioadhesive polymer can be selected from poly(acrylic acid), tragacanth, poly(methylvinylether comaleic anhydride), poly(ethylene oxide), methyl-cellulose, sodium alginate, hydroxypropylmethylcellulose, karaya gum, methylethyl cellulose (and cellulose derivatives such as Metolose), soluble starch, gelatin, pectin, poly(vinyl pyrrolidone), poly(ethylene glycol), poly(vinyl alcohol), poly(hydroxyethyl-methacrylate), hydroxypropylcellulose, sodium carboxymethylcellulose or chitosan.

Other suitable bioadhesive polymers are described in U.S. Pat. No. 6,235,313 to Mathiowitz et al., the teachings of which are incorporated herein by reference, and include polyhydroxy acids, such as poly(lactic acid), polystyrene, polyhyaluronic acids, casein, gelatin, glutin, polyanhydrides, polyacrylic acid, alginate, chitosan; polyacrylates, such as poly(methyl methacrylates), poly(ethyl methacrylates), poly butylmethacrylate), poly-(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), and poly(octadecl acrylate); polyacrylam ides; poly(fumaric-co-sebacic)acid, poly(bis carboxy phenoxy propane-co-sebacic anhydride), polyorthoesters, and copolymers, blends and mixtures thereof.

In certain embodiments, the bioadhesive is an alginate. Alginic acid and its salts associates with sodium and potassium bicarbonate have shown that, after entering a more acidic environment they form a viscous suspension (or a gel) exerting protecting activity over gastric mucosa. These properties are readily adaptable for topical delivery to the esophagus, particularly the lower esophagus. The scientific and patent literature on its activity is wide. Thus, for example, for delivery to the esophagus: Mandel K. G.; Daggy B. P.; Brodie D. A; Jacoby, H. L., 2000. Review article: Alginate-raft formulations in the treatment of heartburn and acid reflux. Aliment. Pharmacol. Ther. 14 669-690, which is incorporated by reference herein in its entirety; and Bioadhesive esophageal bandages: protection against acid and pepsin injury. Man Tang, Peter Dettmar, Hannah Batchelor—International Journal of Pharmaceutics 292 (2005)—169-177, which is incorporated by reference herein in its entirety.

In certain embodiments, the bioadhesive is a bioadhesive hydrogel. Bioadhesive hydrogels are well known in art and suitable hydrogels that be used for delivery of the anti-PESC agents of the present disclosure are described in a wide range of scientific and patent literature on its activity is wide. An exemplary hydrogel formulation is described in Collaud et al. “Clinical evaluation of bioadhesive hydrogels for topical delivery of hexylaminolevulinate to Barrett's esophagus” J Control Release. 2007 Nov. 20; 123(3):203-10.

a. Bioadhesive Microparticle Formulations

In certain embodiments, the anti-PESC agent (optionally with other active agents) are formulated into adhesive polymeric microspheres have been selected on the basis of the physical and chemical bonds formed as a function of chemical composition and physical characteristics, such as surface area, as described in detail below. These microspheres are characterized by adhesive forces to mucosa of greater than 11 mN/cm² on esophageal tissue. The size of these microspheres can range from between a nanoparticle to a millimeter in diameter. The adhesive force is a function of polymer composition, biological substrate, particle morphology, particle geometry (e.g., diameter) and surface modification.

Suitable polymers that can be used to form bioadhesive microspheres include soluble and insoluble, biodegradable and nonbiodegradable polymers. These can be hydrogels or thermoplastics, homopolymers, copolymers or blends, natural or synthetic. The preferred polymers are synthetic polymers, with controlled synthesis and degradation characteristics. Most preferred polymers are copolymers of fumaric acid and sebacic acid, which have unusually good bioadhesive properties when administered to the gastrointestinal.

In the past, two classes of polymers have appeared to show useful bioadhesive properties: hydrophilic polymers and hydrogels. In the large class of hydrophilic polymers, those containing carboxylic groups (e.g., poly[acrylic acid]) exhibit the best bioadhesive properties. One could infer that polymers with the highest concentrations of carboxylic groups should be the materials of choice for bioadhesion on soft tissues. In other studies, the most promising polymers were sodium alginate, carboxymethylcellulose, hydroxymethylcellulose and methylcellulose. Some of these materials are water-soluble, while others are hydrogels.

Rapidly bioerodible polymers such as poly[lactide-co-glycolide], polyanhydrides, and polyorthoesters, whose carboxylic groups are exposed on the external surface as their smooth surface erodes, are excellent candidates for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers include proteins, such as zein, modified zein, casein, gelatin, gluten, serum albumin, or collagen, and polysaccharides, such as cellulose, dextrans, polyhyaluronic acid, polymers of acrylic and methacrylic esters and alginic acid. These are not preferred due to higher levels of variability in the characteristics of the final products, as well as in degradation following administration. Synthetically modified natural polymers include alkyl celluloses, hydroxyalkyl celluloses, cellulose ethers, cellulose esters, and nitrocelluloses.

Representative synthetic polymers include polyphosphazines, poly(vinyl alcohols), polyam ides, polycarbonates, polyalkylenes, polyacrylam ides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

In some instances, the polymeric material could be modified to improve bioadhesion either before or after the fabrication of microspheres. For example, the polymers can be modified by increasing the number of carboxylic groups accessible during biodegradation, or on the polymer surface. The polymers can also be modified by binding amino groups to the polymer. The polymers can also be modified using any of a number of different coupling chemistries that covalently attach ligand molecules with bioadhesive properties to the surface-exposed molecules of the polymeric microspheres.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of bioadhesive ligands to the polymeric microspheres described herein. Any polymer that can be modified through the attachment of lectins can be used as a bioadhesive polymer for purposes of drug delivery or imaging.

Lectins that can be covalently attached to microspheres to render them target specific to the mucin and mucosal cell layer could be used as bioadhesives. Useful lectin ligands include lectins isolated from: Abrus precatroius, Agaricus bisporus, Anguilla anguilla, Arachis hypogaea, Pandeiraea simplicifolia, Bauhinia purpurea, Caragan arobrescens, Cicer arietinum, Codiurn fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The attachment of any positively charged ligand, such as polyethyleneimine or polylysine, to any microsphere may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most microspheres with the appropriate chemistry, such as CDI, and be expected to influence the binding of microspheres to the gut. For example, polyclonal antibodies raised against components of mucin or else intact mucin, when covalently coupled to microspheres, would provide for increased bioadhesion. Similarly, antibodies directed against specific cell surface receptors exposed on the lumenal surface of the intestinal tract would increase the residence time of beads, when coupled to microspheres using the appropriate chemistry. The ligand affinity need not be based only on electrostatic charge, but other useful physical parameters such as solubility in mucin or else specific affinity to carbohydrate groups.

The covalent attachment of any of the natural components of mucin in either pure or partially purified form to the microspheres would decrease the surface tension of the bead-gut interface and increase the solubility of the bead in the mucin layer. The list of useful ligands would include but not be limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range would yield chains of 120 to 425 amino acid residues attached to the surface of the microspheres. The polyamino chains would increase bioadhesion by means of chain entanglement in mucin strands as well as by increased carboxylic charge.

As used herein, the term “microspheres” includes microparticles and microcapsules (having a core of a different material than the outer wall), having a diameter in the nanometer range up to 5 mm. The microsphere may consist entirely of bioadhesive polymer or have only an outer coating of bioadhesive polymer.

As characterized in the following examples, microspheres can be fabricated from different polymers using different methods. Polylactic acid blank microspheres were fabricated using three methods: solvent evaporation, as described by E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984); hot-melt microencapsulation, as described by E. Mathiowitz, et al., Reactive Polymers, 6, 275 (1987); and spray drying. Polyanhydrides made of bis-carboxyphenoxypropane and sebacic acid with molar ratio of 20:80 P(CPP-SA) (20:80) (Mw 20,000) were prepared by hot-melt microencapsulation. Poly(fumaric-co-sebacic) (20:80) (Mw 15,000) blank microspheres were prepared by hot-melt microencapsulation. Polystyrene microspheres were prepared by solvent evaporation.

In certain embodiments, the composition includes a bioadhesive matrix in which particles (such as nanoparticles) containing the anti-PESC agents are dispersed. In these embodiments, the bioadhesive matrix promotes contact between the mucosa of the esophagus and the nanoparticles.

In certain embodiments, the drug-containing particle is a matrix, such as as a bioerodible, bioadhesive matrix. Suitable bioerodible, bioadhesive polymers include bioerodible hydrogels, such as those described by Sawhney, et al., in Macromolecules, 1993, 26:581-587, the teachings of which are incorporated herein by reference. Representative bioerodible, bioadhesive polymers include, but are not limited to, synthetic polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide), poly(lactide-co-caprolactone), poly(ethylene-co-maleic anhydride), poly(ethylene maleic anhydride-co-L-dopamine), poly(ethylene maleic anhydride-co-phenylalanine), poly(ethylene maleic anhydride-co-tyrosine), poly(butadiene-co-maleic anhydride), poly(butadiene maleic anhydride-co-L-dopamine) (pBMAD), poly(butadiene maleic anhydride-co-phenylalanine), poly(butadiene maleic anhydride-co-tyrosine), poly(fumaric-co-sebacic)anhydride (P(FA:SA)), poly(bis carboxy phenoxy propane-co-sebacic anhydride) (20:80) (poly(CCP:SA)), as well as blends comprising these polymers; and copolymers comprising the monomers of these polymers, and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers, blends and mixtures thereof. In general, these materials degrade either by enzymatic hydrolysis or exposure to water in vivo, by surface or bulk erosion.

Particles having an average particle size of between 10 nm and 10 microns are useful in the compositions described herein. In certain embodiments, the particles are nanoparticles, having a size range from about 10 nm to 1 micron, preferably from about 10 nm to about 0.1 microns. In particularly preferred embodiments, the particles have a size range from about 500 to about 600 nm. The particles can have any shape but are generally spherical in shape.

The compositions described herein contain a monodisperse plurality of nanoparticles. Preferably, the method used to form the nanoparticles produces a monodisperse distribution of nanoparticles; however, methods producing polydisperse nanoparticle distributions can be used. If the method does not produce particles having a monodisperse size distribution, the particles are separated following particle formation to produce a plurality of particles having the desired size range and distribution.

Nanoparticles useful in the compositions described herein can be prepared using any suitable method known in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), coacervation, low temperature microsphere formation, and phase inversion nanoencapsulation (PIN). A brief summary of these methods is presented below.

b. Spray Drying

Methods for forming microspheres/nanospheres using spray drying techniques are described in U.S. Pat. No. 6,620,617, to Mathiowitz et al. In this method, the polymer is dissolved in an organic solvent such as methylene chloride or in water. A known amount of one or more active agents to be incorporated in the particles is suspended (in the case of an insoluble active agent) or co-dissolved (in the case of a soluble active agent) in the polymer solution. The solution or dispersion is pumped through a micronizing nozzle driven by a flow of compressed gas, and the resulting aerosol is suspended in a heated cyclone of air, allowing the solvent to evaporate from the microdroplets, forming particles. Microspheres/nanospheres ranging between 0.1-10 microns can be obtained using this method.

c. Interfacial Polymerization

Interfacial polymerization can also be used to encapsulate one or more active agents. Using this method, a monomer and the active agent(s) are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

d. Hot Melt Microencapsulation

Microspheres can be formed from polymers such as polyesters and polyanhydrides using hot melt microencapsulation methods as described in Mathiowitz et al., Reactive Polymers, 6:275 (1987). In this method, the use of polymers with molecular weights between 3-75,000 daltons is preferred. In this method, the polymer first is melted and then mixed with the solid particles of one or more active agents to be incorporated that have been sieved to less than 50 microns. The mixture is suspended in a non-miscible solvent (like silicon oil), and, with continuous stirring, heated to 5. degree. C. above the melting point of the polymer. Once the emulsion is stabilized, it is cooled until the polymer particles solidify. The resulting microspheres are washed by decanting with petroleum ether to give a free-flowing powder.

e. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

f. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

g. Solvent Evaporation Microencapsulation

Methods for forming microspheres using solvent evaporation techniques are described in E. Mathiowitz et al.,Scanning Microscopy, 4:329 (1990); L. R. Beck et al., Fertil. Steril., 31:545 (1979); L. R. Beck et al Am J Obstet Gynecol 135(3) (1979); S. Benita et al., Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microspheres/nanospheres. This method is useful for relatively stable polymers like polyesters and polystyrene. However, labile polymers, such as polyanhydrides, may degrade during the fabrication process due to the presence of water. For these polymers, some of the following methods performed in completely anhydrous organic solvents are more useful.

h. Solvent Removal Microencapsulation

The solvent removal microencapsulation technique is primarily designed for polyanhydrides and is described, for example, in WO 93/21906 to Brown University Research Foundation. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Microspheres that range between 1-300 microns can be obtained by this procedure. Substances which can be incorporated in the microspheres include pharmaceuticals, pesticides, nutrients, imaging agents, and metal compounds.

i. Coacervation

Encapsulation procedures for various substances using coacervation techniques are known in the art, for example, in GB-B-929 406; GB-B-929 40 1; and U.S. Pat. Nos. 3,266,987, 4,794,000, and 4,460,563. Coacervation involves the separation of a macromolecular solution into two immiscible liquid phases. One phase is a dense coacervate phase, which contains a high concentration of the polymer encapsulant (and optionally one or more active agents), while the second phase contains a low concentration of the polymer. Within the dense coacervate phase, the polymer encapsulant forms nanoscale or microscale droplets. Coacervation may be induced by a temperature change, addition of a non-solvent or addition of a micro-salt (simple coacervation), or by the addition of another polymer thereby forming an interpolymer complex (complex coacervation).

j. Low Temperature Casting of Microspheres

Methods for very low temperature casting of controlled release microspheres are described in U.S. Pat. No. 5,019,400 to Gombotz et al. In this method, a polymer is dissolved in a solvent optionally with one or more dissolved or dispersed active agents. The mixture is then atomized into a vessel containing a liquid non-solvent at a temperature below the freezing point of the polymer-substance solution which freezes the polymer droplets. As the droplets and non-solvent for the polymer are warmed, the solvent in the droplets thaws and is extracted into the non-solvent, resulting in the hardening of the microspheres.

k. Phase Inversion Nanoencapsulation (PIN)

Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non-solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et al. The method can be used to produce monodisperse populations of nanoparticles and microparticles in a wide range of sizes, including, for example, about 100 nanometers to about 10 microns.

Advantageously, an emulsion need not be formed prior to precipitation. The process can be used to form microspheres from thermoplastic polymers.

l. Sequential Phase Inversion Nanoencapsulation (sPIN)

Multi-walled nanoparticles can also be formed by a process referred to herein as “sequential phase inversion nanoencapsulation” (sPIN). This process is described in detail below in Section IV. sPIN is particularly suited for forming monodisperse populations of nanoparticles, avoiding the need for an additional separations step to achieve a monodisperse population of nanoparticles.

m. Dissolving Tablet

In certain embodiments, the anti-PESC agents is provided in a dissolving tablet. For example, the tablet can contain a therapeutically effective amount of the anti-PESC agent in combination with polyvinylpyrrolidone (PVP: povidone), wherein the tablet is formulated to rapidly dissolve in a specific volume of liquid so as to generate a topical esophageal therapy suitable for delivering the anti-PESC to the luminal surface of the esophagus.

For instance, the the volume of liquid in which the tablet dissolves can be from 5 to 50 mL, 5 to 25 mL or even 5 to 15 mL. Preferably the liquid is water.

The dissolving tablet can also further include an excipient that renders the dissolving tablet palatable, especially at least one excipient that increases viscosity of the topical esophageal therapy. An exemplary viscosity-enhancing excipient is mannitol.

n. Topical Formulation

In certain embodiments, the anti-PESC agent is provided in a topical, non-systemic, oral, slow releasing, solid, soft lozenge pharmaceutical composition comprising: (a) about 1% to about 5% by mass of one or more release modifiers comprising polyethylene oxide polymers comprising a molecular weight of about 900,000 to about 8,000,000; (b) about 10% to about 60% by mass of one or more film-forming polymers comprising gelatins; (c) about 5% to about 20% by mass of one or more plasticizers comprising glycerol, sorbitol, or combinations thereof; and (d) less than 1% by mass of one or more anti-PESC agents. Exemplary plasticizers include glycerol, sorbitol, mannitol, maltitol, xylitol, or combinations thereof. The lozenge may also include one or more sweeteners, such as maltitol, xylitol, mannitol, sucralose, aspartame, stevia, or a combination thereof. The lozenge may also include one or more pH modifiers comprising one or more organic acids.

### VII. EXAMPLES

Barrett's Esophagus (BE) is an irreversible condition that is believed to be the precancerous lesion of Esophageal Adenocarcinoma (EAC). BE originates from a unique cell population preexisting at gastroesophageal junction and possesses its own stem cells. Using a novel ground-state stem cell cloning technology, we derived patient-matched stem cell pedigree lines from BE and Esophageal epithelia. Image-based high-throughput chemical screening was developed and used to uncover a unique combination of chemicals that can specifically eradicate BE stem cells while protecting and promoting Esophageal stem cells. These effects were confirmed in a co-culture 3D model and a mouse xenograft model by co-transplanting BE and Esophageal stem cells in vivo. Interestingly, this drug combination was also able to eradicate stem cells in patient-matched dysplasia and cancer. It is the first time that ground-state stem cells from patient-matched BE, dysplasia, cancer and normal tissue can be cloned and expanded in vitro and employed to identify targeted therapeutics for not only preemptive therapies of BE but therapies targeting late-stage cancers. It is anticipated that this platform can be applied to provide the promise of developing novel strategies for chemoprevention and treatment of various types of lethal cancers.

### Cloning Barrett's Esophagus Stem Cells and Chemical Screening

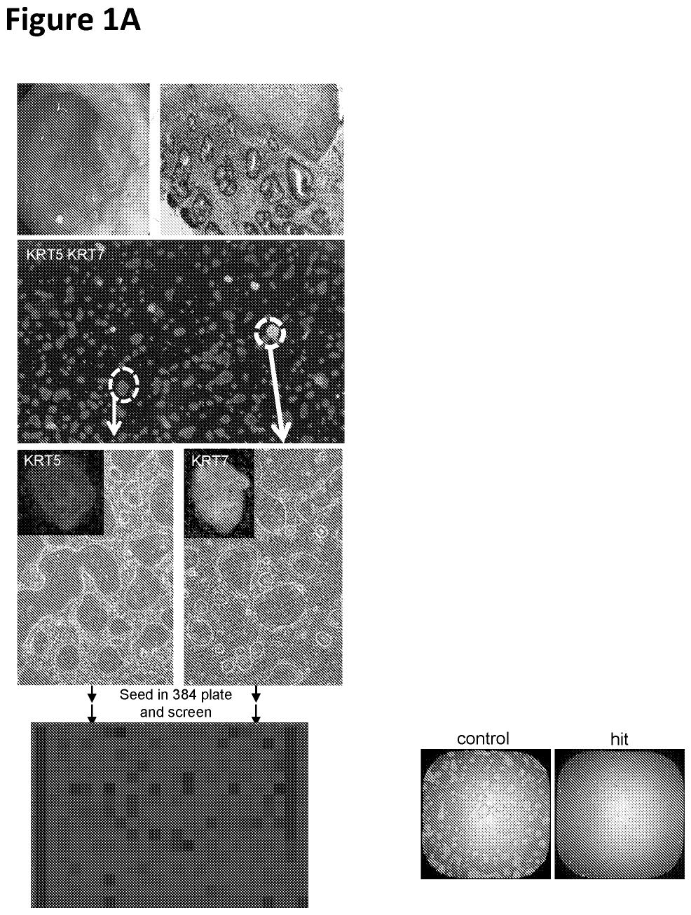

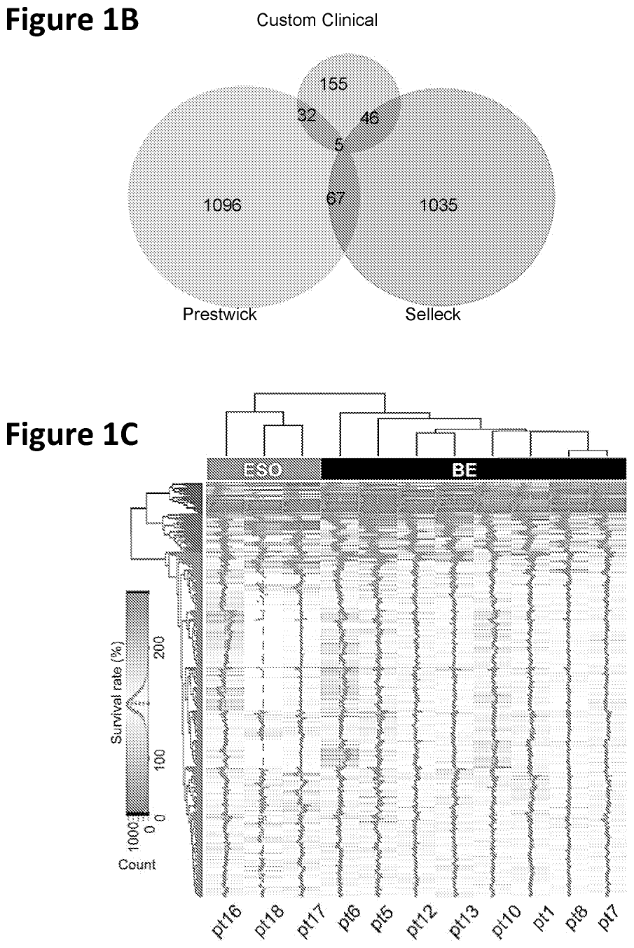

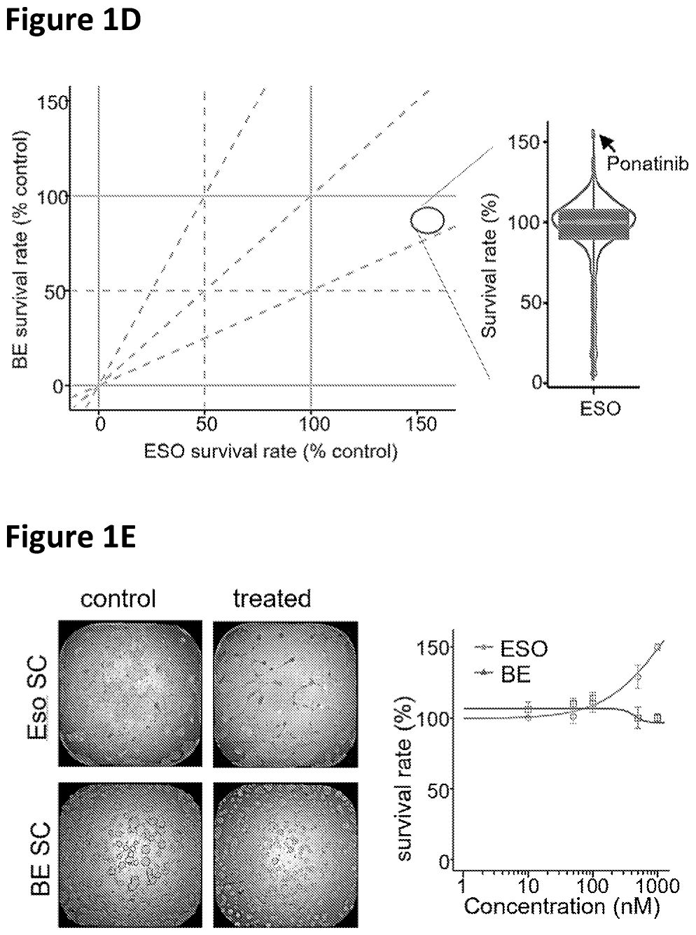

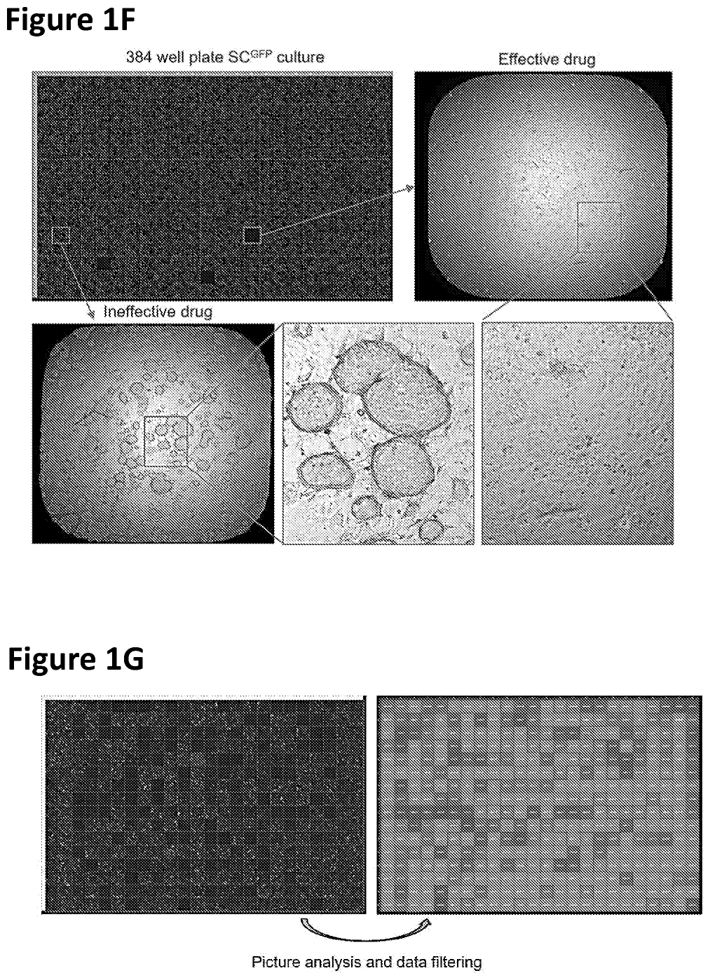

Endoscopic mucosal biopsies obtained from the distal esophagus of Barrett's patients can include Esophageal squamous epithelium and Barrett's Esophagus (FIG. 1A; Yamamoto et al). Colonies arose from single cell suspensions of these 1 mm biopsies one week after plating onto lawns of irradiated 3T3 cells in growth media known to support immature, epithelial stem cells (Wang et al., 2015; Yamamoto et al., 2016; Duleba et al., 2018) (FIG. 1A; StemECHO, MCT). As reported previously (Yamamoto et al., 2016), the colonies yielded from Barrett's yielded mixtures of both Krt5-positive clones typical of the esophageal squamous basal cells and ones that expressed the columnar epithelial marker Krt7 (FIG. 1A). To separate these two populations of clones derived from the Barrett's biopsies, multiple single colonies were samples and expanded as independent pedigrees (Yamamoto et al., 2016) (FIG. 1A). Reprobing these pedigree lines with the same antibodies showed that the original Barrett's biopsies contained two distinct clonogenic cells marked by committed expression of either Krt5 or Krt7 (Fig.1A).
In order to explore the possibility of uncovering an agent that can selectively eradicate Barrett's stem cells, a high throughput chemical screening on a panel of esophageal squamous stem cells and Barrett's stem cells was set up. Three libraries were used for this screening including Custom Clinical, Prestwick and Selleck in totally of non-overlapping 2276 chemicals (FIG. 1B). While approximately 70% of the chemicals had no impact on either of the cell lines and around 10% of the chemicals eradicate both cell types, the other 20% of the chemicals displayed differential efforts on these two cell types (FIGS. 1C, 1F and 1G). The selected hits were validated in a dosage-response manner, suggesting the reliability of the screening approach (FIGS. 1F and 1G). Among these 20% of the chemicals, we did not observe one single agent that can play dual roles including eradication of Barrett's stem cells and protection of esophageal stem cells. Interestingly, Ponatinib was identified to significantly increase the proliferation of esophageal stem cells while slightly decreasing the survival of Barrett's stem cells (FIGS. 1D and E).

### Synthetic Chemical Screening for Dual Action Regime

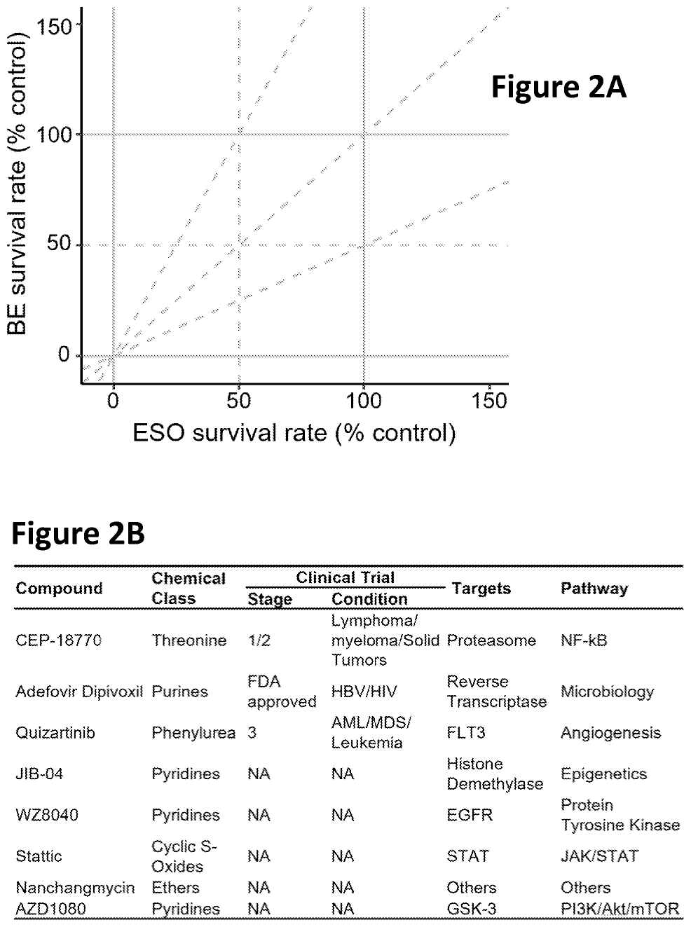

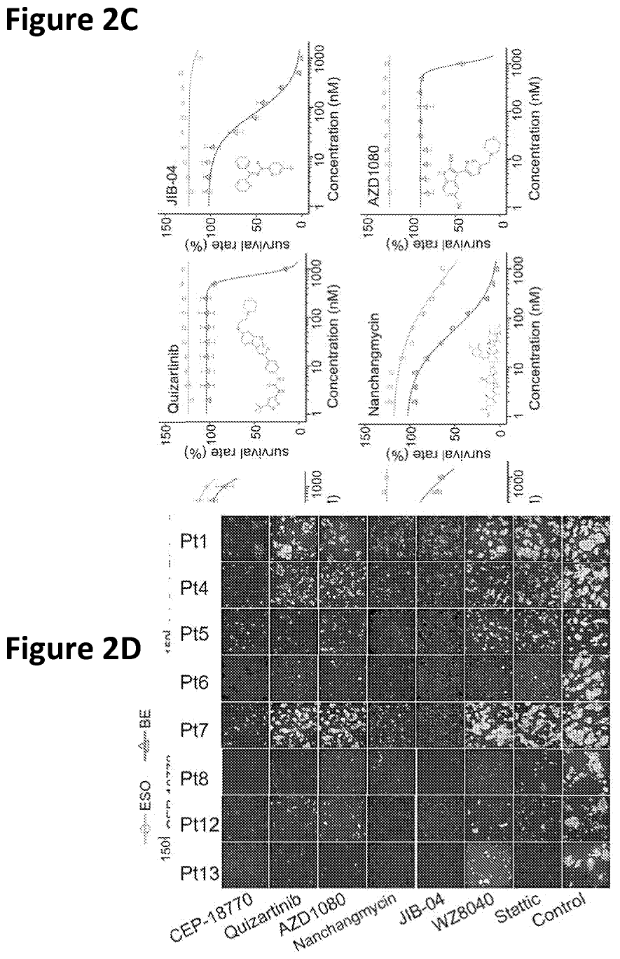

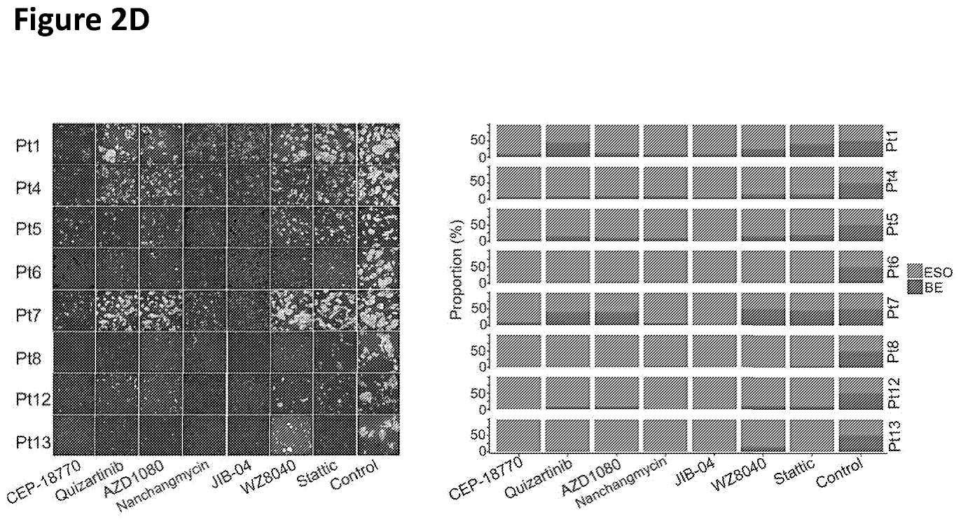

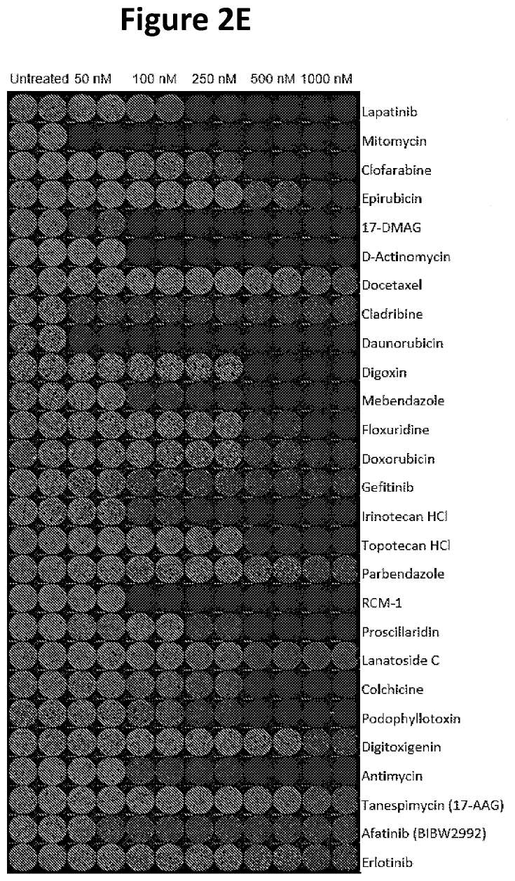

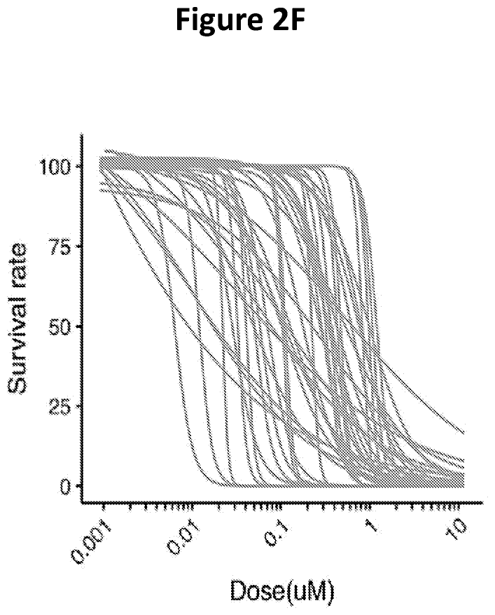

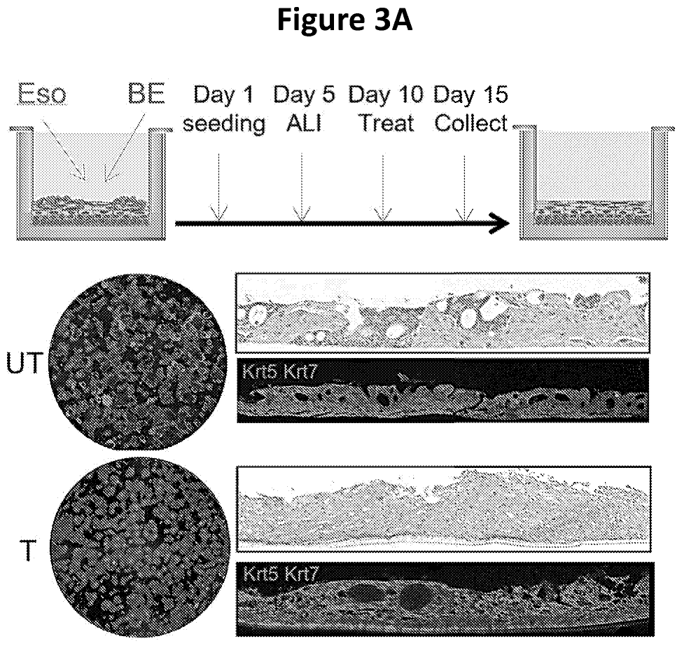

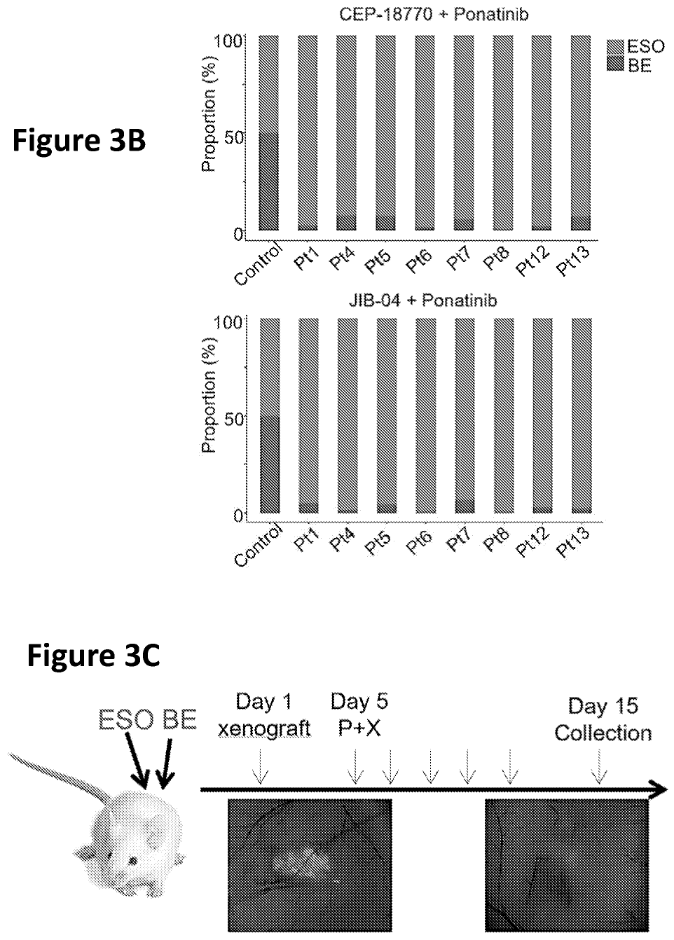

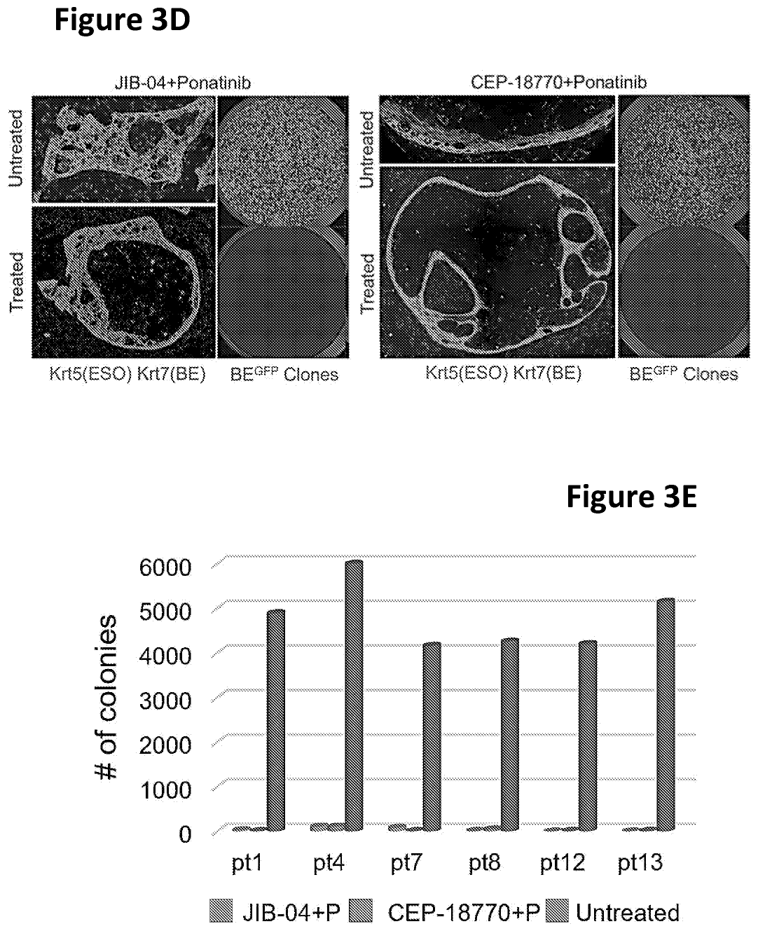

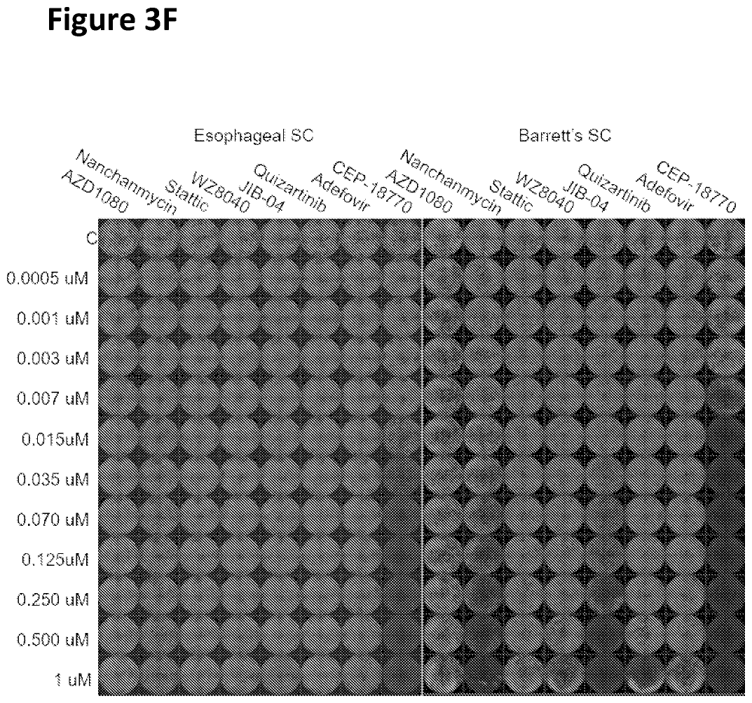

It was next hypothesized that one chemical in our libraries could function with Ponatinib in a synergistic manner to specifically eradicate Barrett's stem cells while protecting and promoting esophageal stem cells. The same libraries were screened in the background of 1 μM Ponatinib. Interestingly, eight compounds displayed the lethality towards Barrett's stem cells without affecting Esophageal stem cells in the presence of Ponatinib (FIG. 2A). Among them, Adefovir Dipivoxil is a FDA approved drug, CEP-18770 and Qizartinib are in clinical trials. JIB04, WZ8040, Stattic, Nanchangmycin and AZD1080 are not in any clinical trials (FIG. 2B). None of these chemicals have been reported to target BE and interestingly, they are targeting various different pathways and harbor distinct chemical structures. Their differential effects on Barrett's stem cells and Esophageal stem cells were validated in a dosage dependent manner (FIG. 2C; FIG. 3F). Moreover, co-culture of nine Barrett's stem cell lines from patients with spectrum of mutation profiles (Yamamoto et al, 2016) and Esophageal stem cells demonstrated CEP-18770, JIB04, in the presence of Ponatinib displayed uniform lethality to all Barrett's stem cells that we tested while promoting Esophageal squamous stem cells (FIG. 2D). This result suggests that these two chemicals could work synergistically with Ponatinib to target Barrett's stem cells specifically independent of the genomics and stage of these Barrett's stem cells. The combination of Nanchangmycin and Ponatinib was not pursued due to the high lethality of 3T3-J2 feeder cells during the treatment, which suggests potential toxicity towards other types of somatic cells in vivo.

### Validating Dual Action Regime in 3D Culture and Mouse Xenograft Model

A 3D culture model was generated to mimic Barrett's Esophagus in vitro. The esophageal squamous stem cells and Barrett's stem cells were co-cultured in the transwell insert. Following the creation of air-liquid interface, it was observed that the BE islands located among well-differentiated squamous epithelium that well recapitulated the histology of human BE in the patient. Given the similarity of this artificial model and human pathology, the hits were tested in this system to further validate them. Eight Barrett's stem cell lines were co-cultured with Esophageal stem cells in ALI system and CEP-18770 or JIB04 together with Ponatinib were added in the medium following the creation of ALI. The esophageal squamous stem cells were labeled with red fluorescent protein (RFP) while Barrett's stem cells were labeled with green fluorescent protein (GFP). Without the treatment, it was observed that a mixture of GFP and RFP labeled cells in culture. However, following the treatment of either CEP-18770 or JIB04 in the presence of Ponatinib, the disappearance of GFP labeled Barrett's stem cells was observed. The cross sections of the ALI structures showed intact esophageal squamous epithelium without any existence of Barrett's epithelium (FIGS. 2A&B).

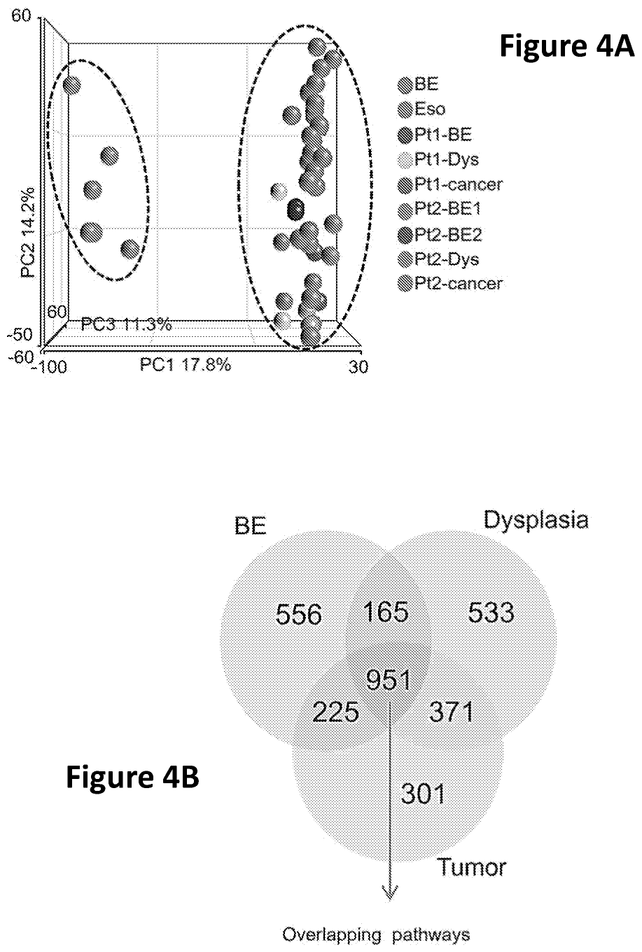

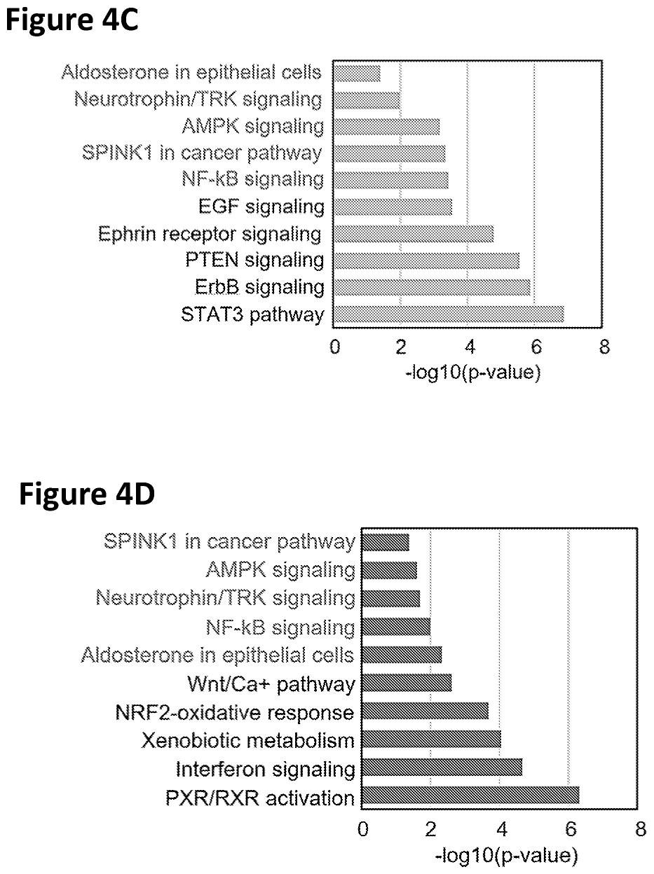

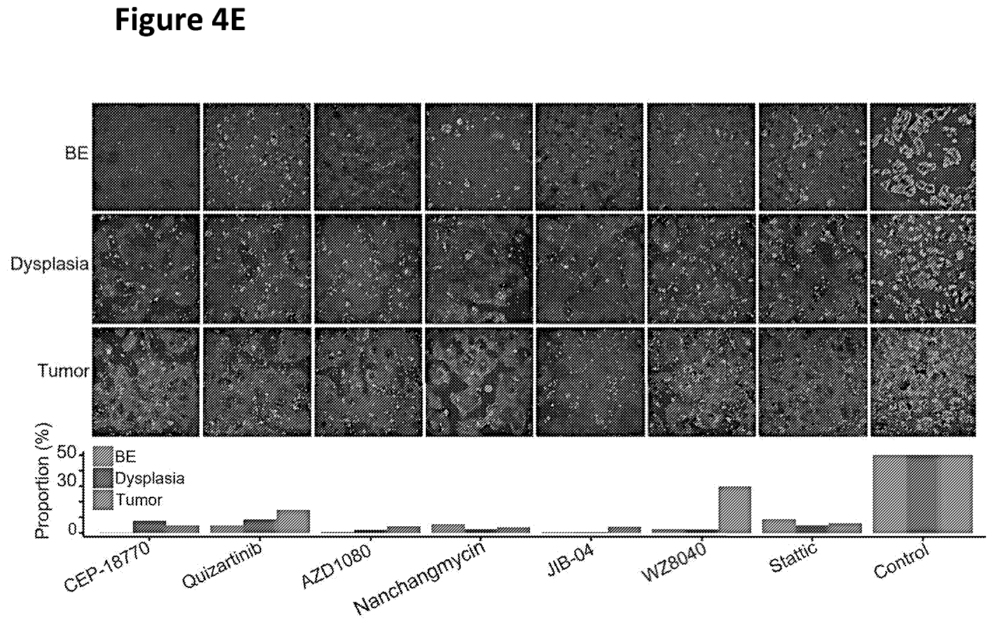

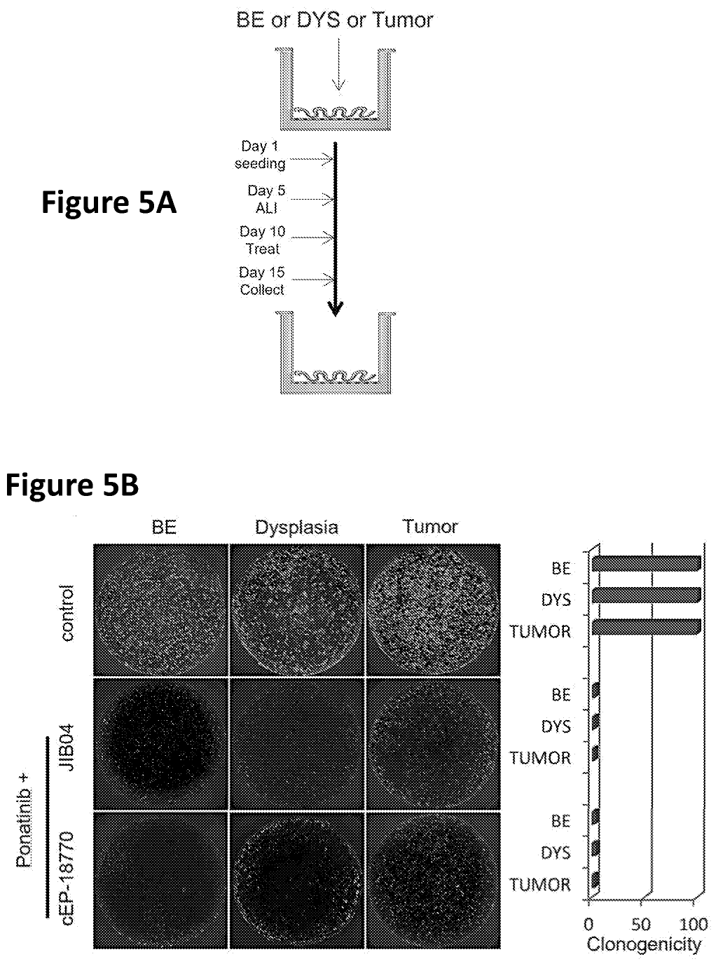

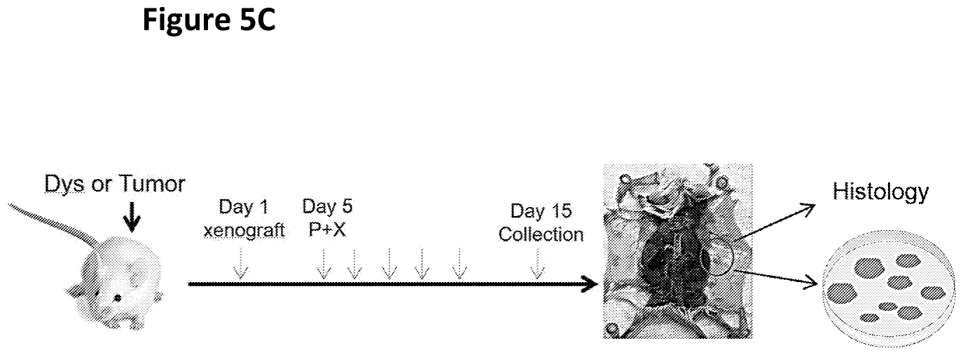

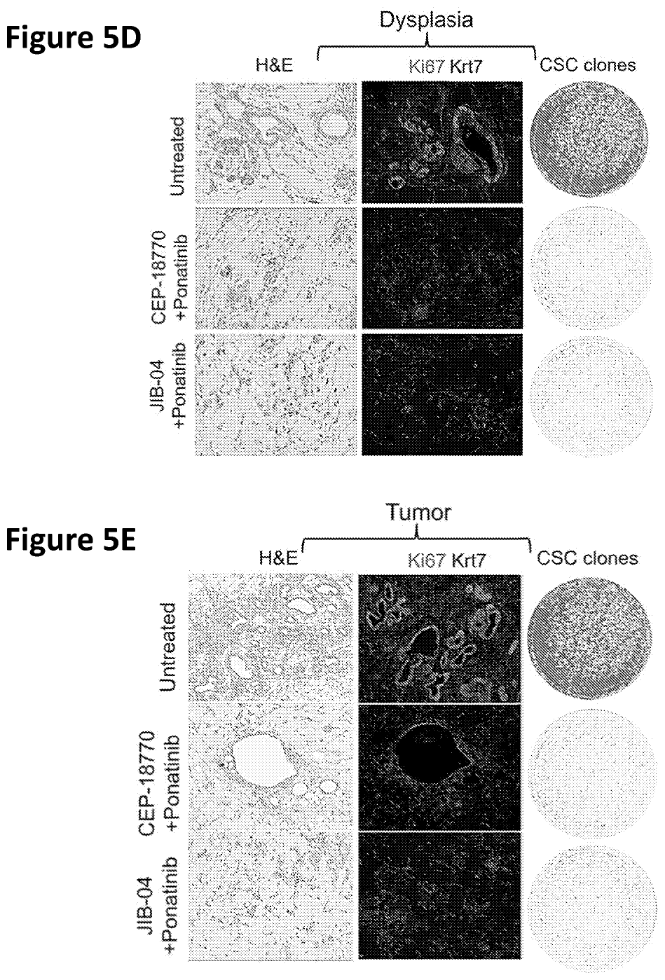

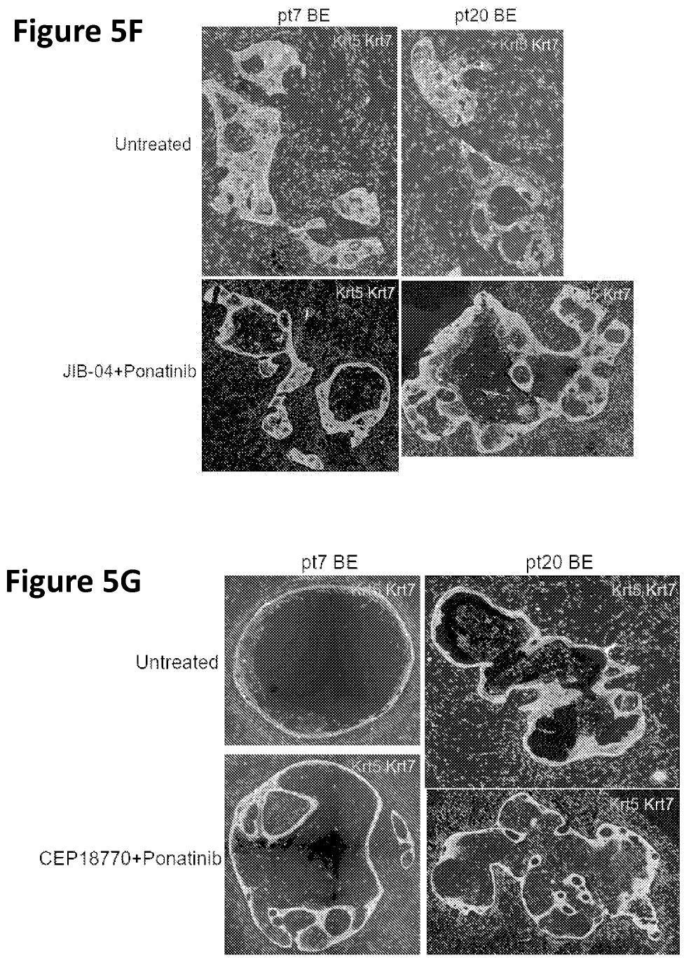

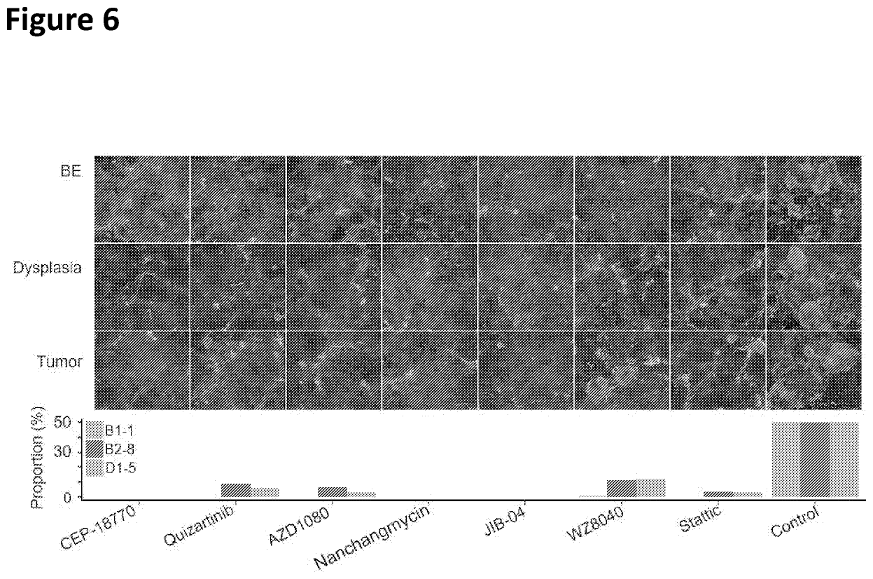

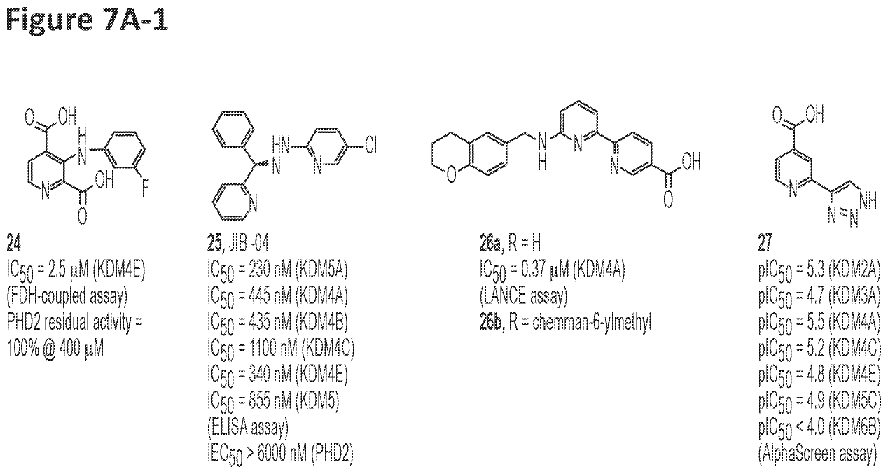

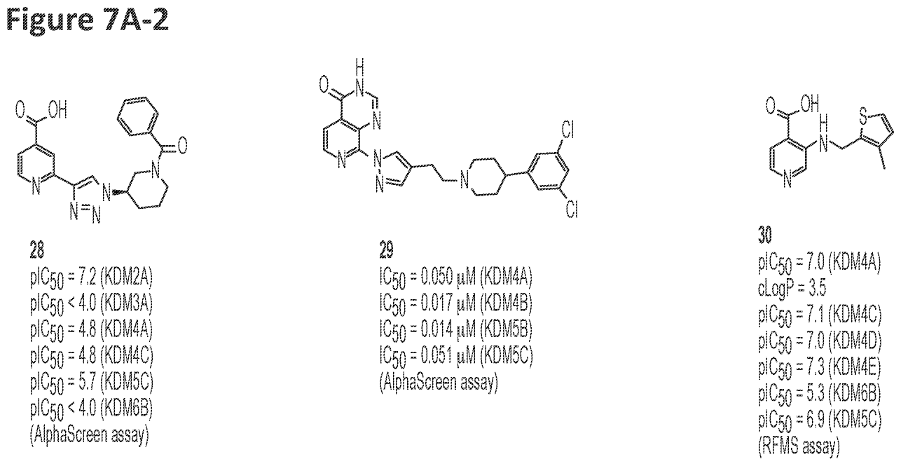

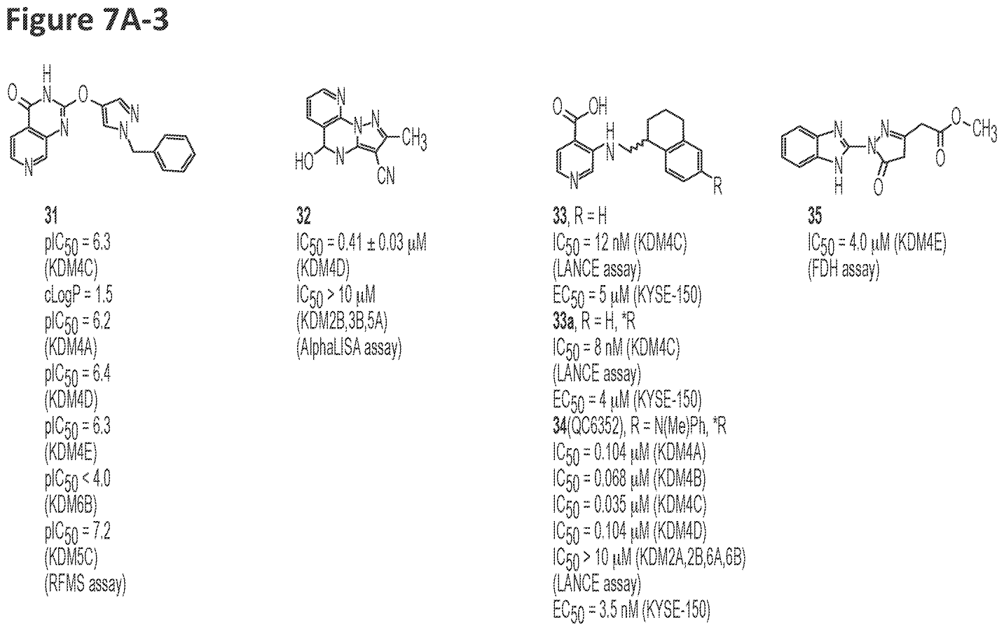

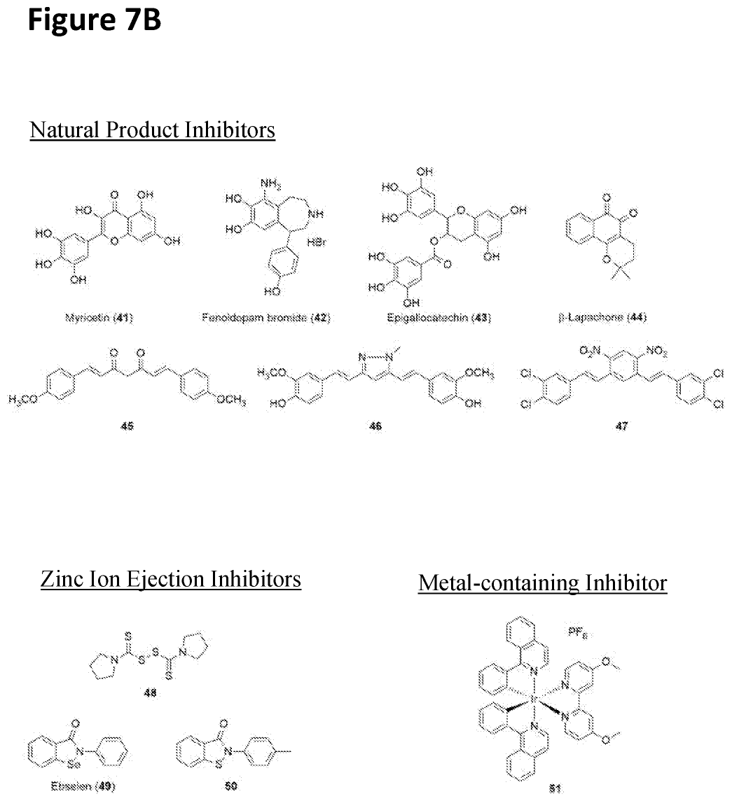

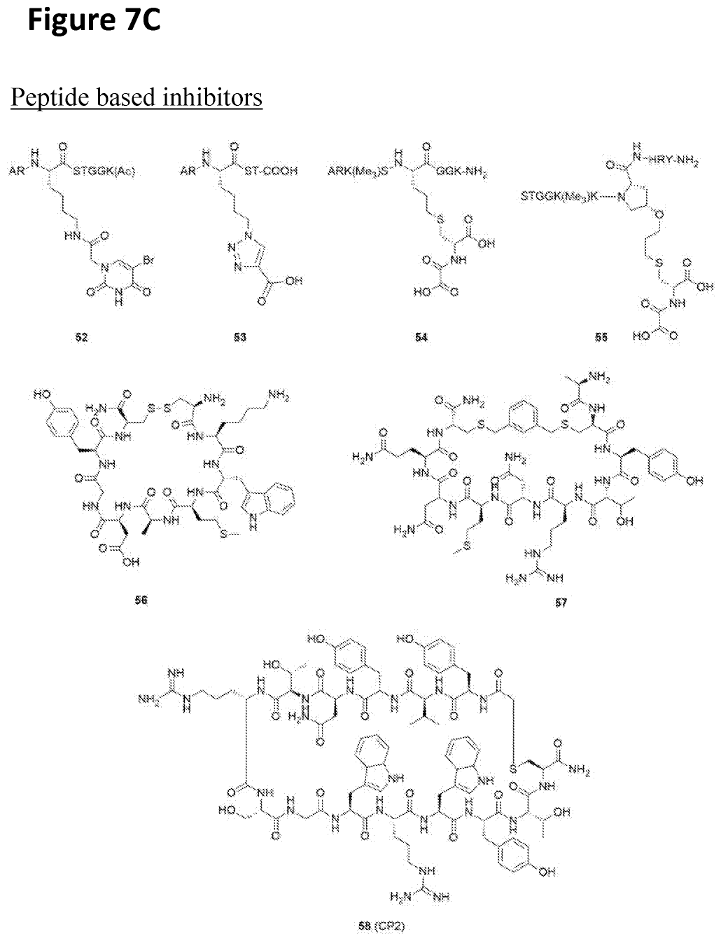

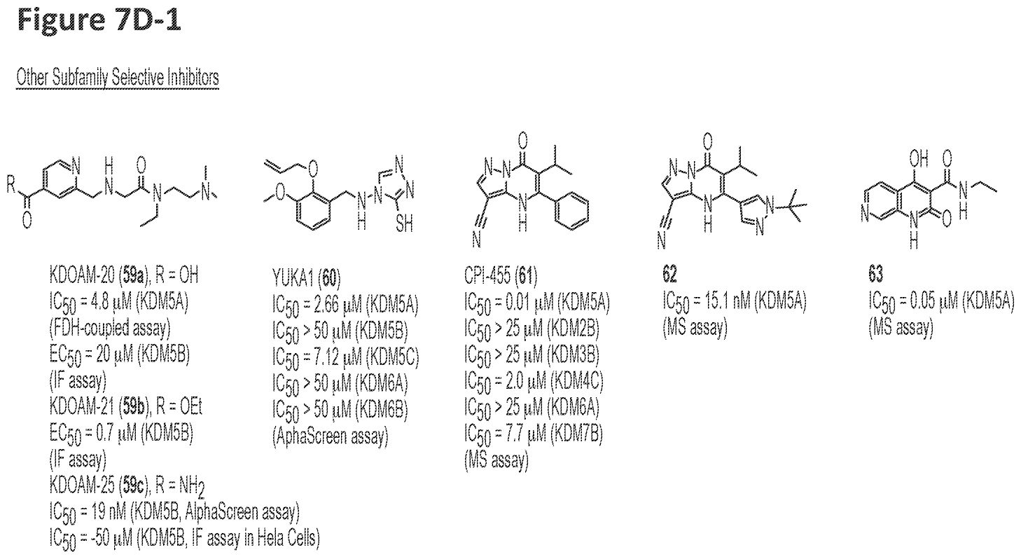

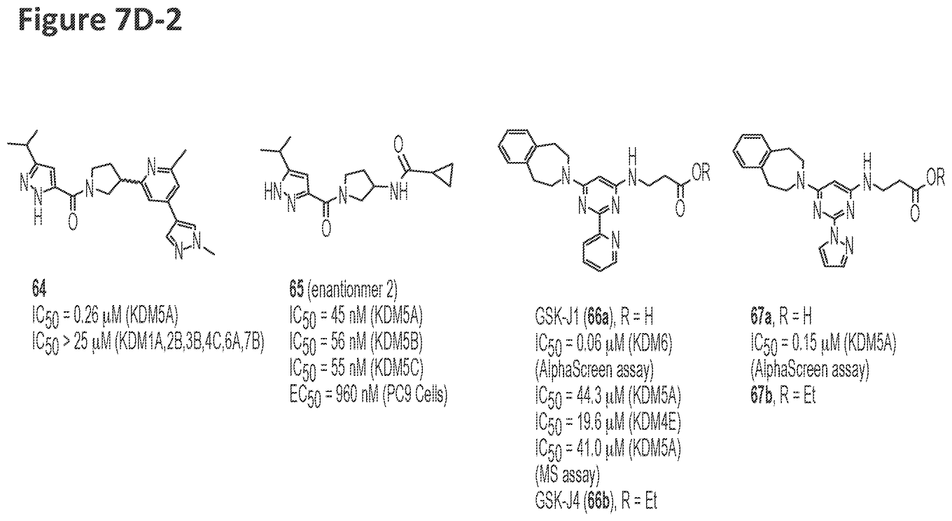

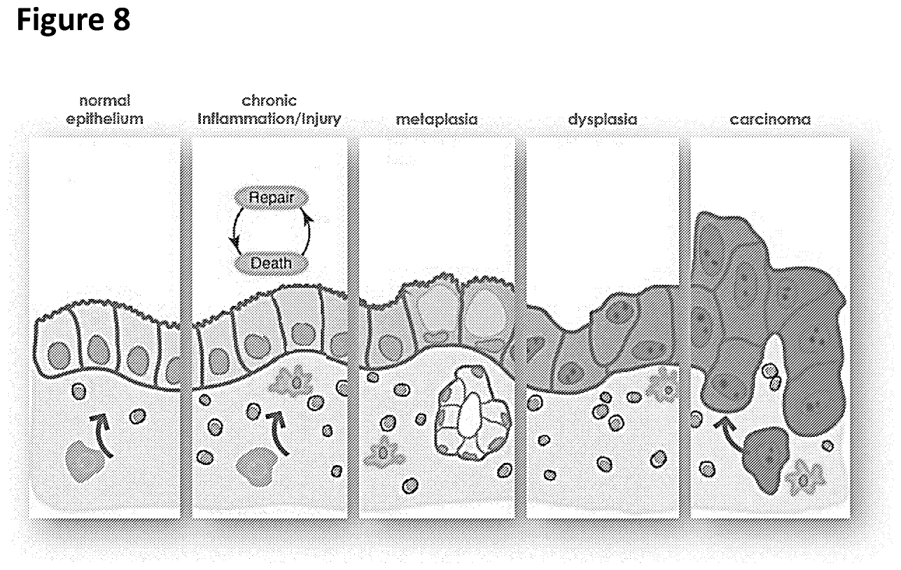

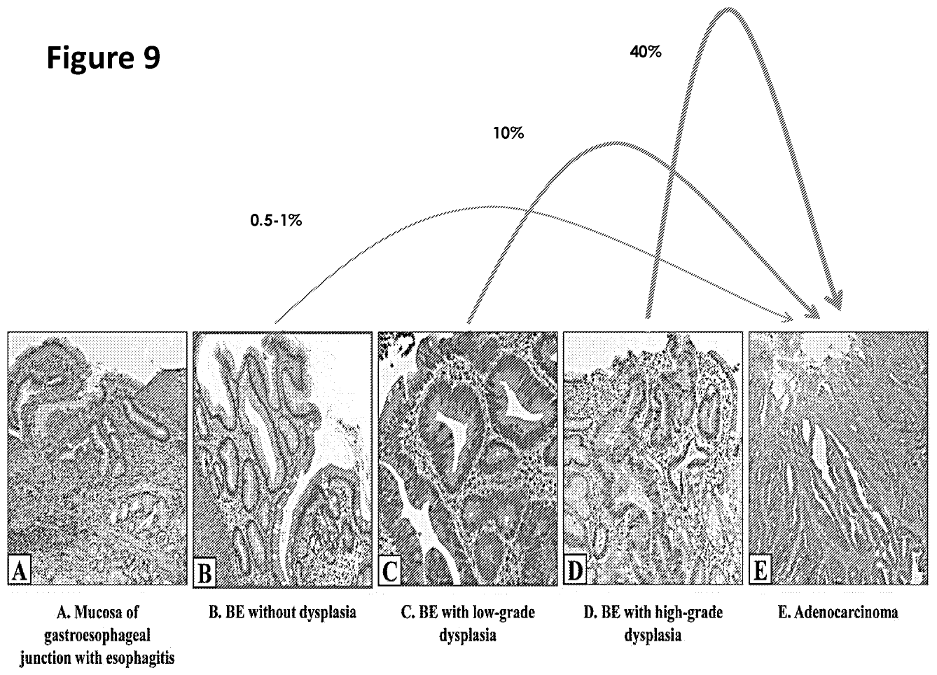

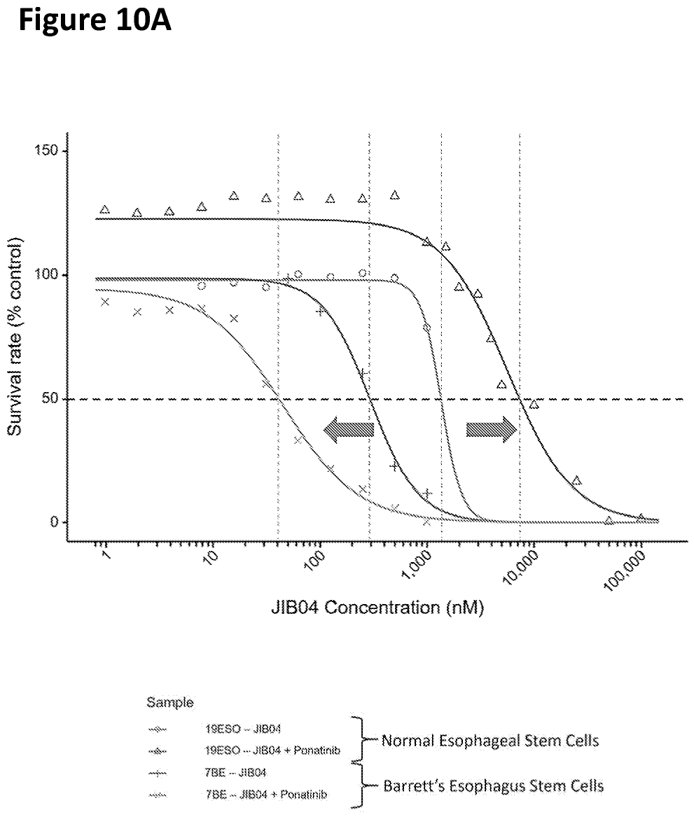

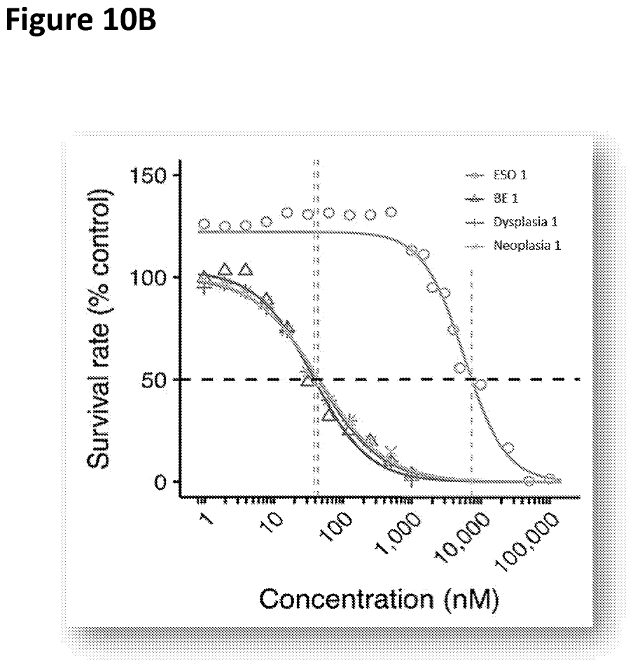

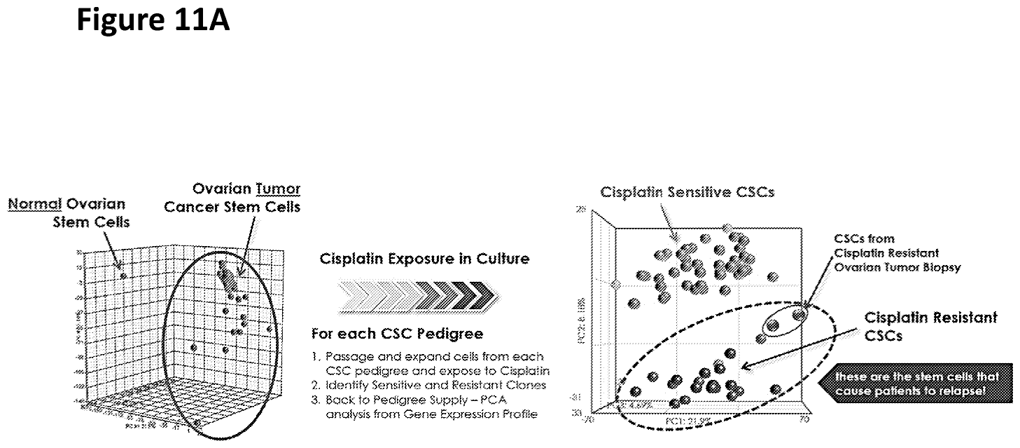

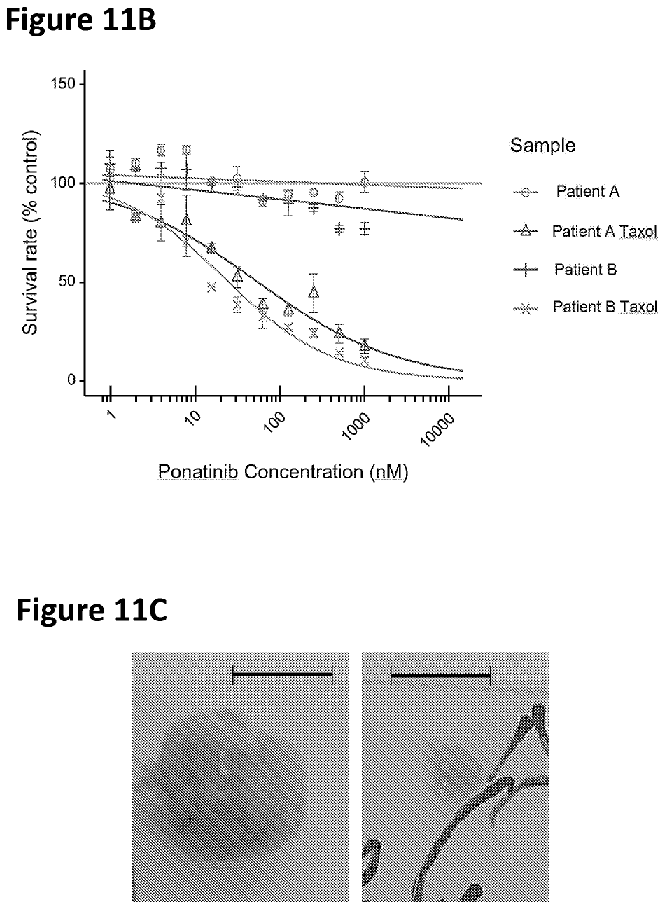

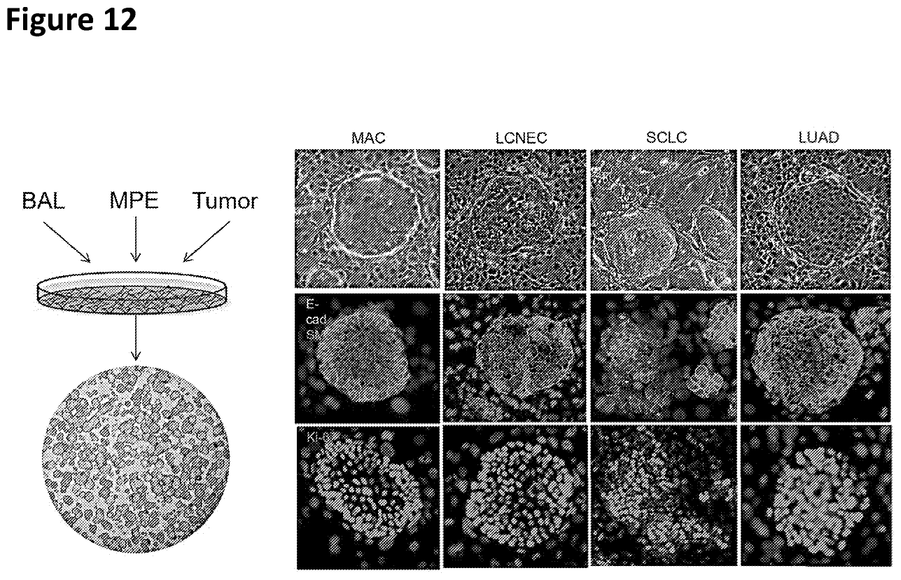

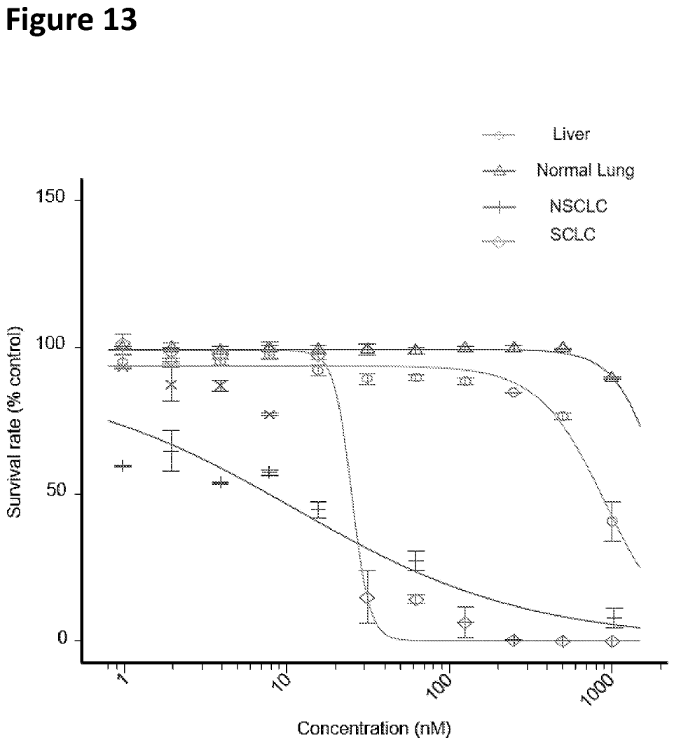

The effect of these chemicals on BE in a xenograft model was further examined. The esophageal squamous stem cells and GFP labeled Barrett's stem cells were mixed and co-injected into the immunodeficient mice. After five days of xenograft, the Ponatinib and CEP-18770 combination or Ponatinib and JIB04 combination was injected into the mice intraperitoneally in an alternative manner (FIG. 3C). Two weeks following the treatment, the xenografts were collected and examined through histology analysis and clonogenic analysis. It was found that the total loss of Barrett's structures in the treated animals, which is consistent with the lack of clonogenic Barrett's stem cells in vitro (FIGS. 3D and 3E; FIG. 4E; FIGS. 5F & 5G).

### Stem Cells of Barrett's Esophagus, Dysplasia and Cancer can be Targeted Similarly

BE has been known to be the precursor of the dysplastic and cancerous lesions of EAC. The transcriptome analysis of patient-matched Barrett's, dysplastic and EAC stem cells revealed significantly overlapping genes and pathways in comparison to the stem cells derived from Esophagus squamous epithelium. Moreover, PCA analysis of Barrett's stem cells derived from 12 patients together with dysplasia, cancer and Esophageal squamous stem cells confirmed that the stem cells of dysplasia and cancer shared very similar gene expression with Barrett's stem cells (FIGS. 4A & B). This data suggests that the strategies developed to eradicate BE could also be used to target dysplasia and EAC which have routinely relapsed from the traditional chemotherapies. The effects of the eight drug hits in combination with Ponatinib were tested in the cell culture of co-seeding esophageal stem cells and Barrett's, dysplasia or EAC stem cells. Most of these combinations could eliminate all three entities without affecting esophageal squamous stem cells in two independent patients (FIG. 4C). Importantly, the JIB04 and CEP-18770 can function in a synergistic manner with Ponatinib to eradicate precancerous, dysplastic and cancerous stem cells of EAC in both 3D culture system and mouse xenograft models (FIG. 5). Given these three entities always co-exist in patients with EAC, our strategies of eradicating all of them could help to eliminate the chance of recurrence of EAC patients drastically.

### Discussion of Results

Despite its influential position in cancer prevention, and fundamental advances in ablative approaches aiming to eradicate it preemptively, Barrett's remains an enormous and growing problem with an estimated 3 million cases in the US alone. Advanced BE lesions that are treated by ablative therapies recur at alarming rates (reference), suggesting an unmet medical need for effective and targeted therapeutic options for these patients. The ability to clone and expand the ground-state stem cells of BE, dysplasia, EAC and normal esophageal squamous epithelium from matched patient samples provides a very promising platform for high-throughput chemical screening devoted to developing highly selective means of eradicating BE and more advanced lesions ahead of the onset of cancer. Each of these stem cell clones can be individually differentiated by polarization in so-called “air-liquid interface” cultures to yield 3-D epithelia remarkably similar to that of the in situ normal, lesional or cancerous epithelia. These same stem cell clones were used in advanced co-culture models with normal epithelial stem cells to investigate the potential ability of selected drug combinations to alter the competitive status of such lesions in the distal esophagus. Furthermore, a novel mouse model for testing drug combinations in vivo was established by transplanting patient-derived BE, dysplasia, EAC and esophageal squamous stem cells in the NSG mice subcutaneously.

The first direction of chemical screening focused on single agents that showed a greater effect against BE stem cells than the patient-matched esophageal stem cells as that was a key goal of this effort. However, upon detailed dose-response studies of these single agent “hits”, the standard differential at the optimal dosing was, at best, 10-fold in concentration. This 10-fold differential in concentration between lethal effects on BE stem cells and those of normal esophagus was disappointing in that it suggested a relatively narrow therapeutic window.

The potential of the small molecules that selectively favored the growth of the normal esophageal stem cells was explored. The intestinal metaplasia of BE is thought to be in competition with the surrounding esophageal mucosa during its proximal spread (Wang et al., 2011). Thus, in any therapeutic strategy, it is possible that this competitive interaction could be exploited from the standpoint of improving the competitive edge of the esophagus. Therefore, it is hypothesized that the molecules found to promote the growth of esophageal stem cells, such as Ponatinib could be used in combination with molecules that limit BE stem cells. Established herein are “synthetic lethal” screens (McCormick, 2015; Thompson et al., 2017; Aguirre and Hahn, 2018) against BE stem cells using small molecule libraries in a background of Ponatinib. Interestingly, this synthetic lethal screen identified a different set of small molecules that, in the context of Ponatinib, efficiently kill BE stem cells while at the same time augmented the growth of normal esophageal stem cells. Among them, JIB04 and CEP-18770 in a synergistic manner with Ponatinib were most effective in eradicating BE stem cells of a wide-range of patients. Most important from the standpoint of potential therapeutics, dose-response curves for these anti-BE stem cell components of this combination, in the background of a fixed concentration of the Ponatinib, shows a remarkable differential of nearly 1,000-fold.

Significant progress has been made in mimicking the competitive interactions between BE and esophageal stem cells through the generation of co-cultures in vitro. Thus, “squamous islands” (Sharma et al., 1998) of esophageal epithelia interspersed with regions dominated by Barrett's esophagus can be generated. These steady state co-cultures have been used to test the impact of the synthetic lethal drug combinations identified in small molecule screens. Significantly, these drug combinations have proven to be remarkably effective in eliminating the Barrett's esophagus and permitting the esophageal stem cells to expand to fill in the regions vacated by the loss of the BE epithelia. Similar efforts were also made to model the competitive interactions between Barrett's and esophageal epithelia in vivo in xenografts. Remarkably, these two drug combinations showed effects on dysplasia and EAC stem cells with similar selectivity and doses as those towards Barrett's stem cells. The large differential between doses of these two effective combinations that eliminated BE, dysplasia, and EAC stem cells versus normal esophageal stem cells suggest the possibility that such drug combinations could produce larger “therapeutic windows” than we observed for single agents directed at the Barrett's alone.

Therefore, in addition to identifying synthetic lethal drug combinations effective for BE, these compounds are shown to also be effective for targeting progenitor cells of dysplasia and EAC that were not part of the initial screen. This finding raises the question as to whether these compounds are targeting some feature of the Correa sequence “lineage” rather than traditional chemotherapeutics that target DNA synthesis or common activities of a rapidly dividing cell.

Taken together, precancerous and cancerous lesions are regenerative and like normal regenerative epithelia depend on discrete populations of immature stem cells for this regenerative growth, and that therapeutics directed at these stem cell populations could be highly specific and effective approaches to eliminating these lesions for therapeutic benefit. Therefore, this platform and similar approaches can be applied to drug discovery efforts in other cancer types.

### Methods

### High Throughput Screening and Imaging of Selected Cell Lines

The GFP-tagged cell lines will be seeded on multiple 384 well plates (Griener Bio-One, USA) (varying according to number of compounds in a library) with a feeder layer of irradiated 3T3-J2 fibroblast cells. The stem cell will be allowed to grow until they divide to become 4-5 cells within a colony. Afterwards they will be transported for treatment with selected chemical library (1 μM) to High Throughput Research and Screening Center at Institute of Biosciences and Technology (Houston, Tex.), Texas A&M University. Positive and negative control lanes will be allocated within each plate. A highly potent drug will be used as a positive control and negative control will be just with DMSO since most of the drugs are dissolved in DMSO. The cells will be allowed to grow for 6 days at 37° C., 7.5% CO2 incubator after treatment. After the cells in the control lanes are at good confluency, the cells will be prepared for imaging. Each multi-well plates will be washed with Phosphate Buffered Saline (Gibco, USA) and fixed with 4% paraformaldehyde at room temperature for 25 minutes. Paraformaldehyde will be replaced with Phosphate Buffered Saline (PBS) and will be imaged using Inverted Eclipse Ti-Series (Nikon, Japan) microscope with Lumencor SOLA light engine, paired with High Content Microscope system (Nikon, Japan), Andor Technology Clara Interline CCD camera, NIS-Elements Advanced Research v.4.13 software (Nikon, Japan) and NIS-Elements HC software (Nikon, Japan). High Content Analysis (HCA) system built on NIS-Elements platform streamlined with automated well plate acquisition and multiple well plate job run will be used for high throughput imaging of phase contrast as well as FITC channel.

### High Content Image Analysis, Selection of Drugs, and Dose-Response Analysis

NIS-Elements High Content Analysis (HCA) system will be used for image data management of multiple well plate job runs. The cells labelled with Green Fluorescent Protein (GFP) in each well of multi well plates will be imaged with features of each stem cell colonies. The changes in cell phenotype compared to control lanes will be measured based on the fluorescent signal threshold using automated image analysis. The features like area, colony number will be exported from the automated analysis. Treated wells will be compared to untreated wells based on GFP-signal area and number of stem cells colonies. The treated well will be normalized with the untreated well based on the area which will be represented in terms of survival rate (percentage of control¹) and will be compared between control and pathogenic population of cells. Z′-factor^{2,3} will be calculated based on the difference between positive and negative control and will be used as criteria for assessing quality of runs. Only plates with Z′-factor>0.6 will be used. The compounds with a coefficient of variation between plate duplicates larger than 20% will be ignored. B-score¹ will be calculated via R package platetools v0.0.2 (github.com/swarchal/platetools) to control the edge and positional bias to help infer the potential hits of inhibitors. Only the compounds with a cutoff of B-score<−2 will be considered as potential inhibitors. The selection of drugs will be made based on the maximum differences in survival rate between patient-matched cell lines (cut-off set at 20%), their targets, structural spectrum, pathways related to them and their possible relation implicated in Barret's esophageal. If there are not patient matched pedigrees, the median value of survival rates in a contain group will be used as representative value for that group of pedigrees. The dose-response curves of survival rate for a certain compound will be calculated by fitting a three-parameter log-logistic dose-response model to the survival rate data using R package dre v3.0.1. R packages Imtest⁵ v0.9-36 and sandwich⁶ v2.4-0 to obtain robust standard errors to address the fact that some variance heterogeneity is present. The ED50 value is estimated by module ED in the R package drc.

### VIII. REFERENCES

1. Malo, N., Hanley, J. A., Cerquozzi, S., Pelletier, J. & Nadon, R. Statistical practice in high-throughput screening data analysis. Nat Biotechnol 24, 167-75 (2006).

2. Zhang, J. H., Chung, T. D. & Oldenburg, K. R. A Simple Statistical Parameter for Use in Evaluation and Validation of High Throughput Screening Assays. J Biomol Screen 4, 67-73 (1999).

3. Iversen, P. W., Eastwood, B. J., Sittampalam, G. S. & Cox, K. L. A comparison of assay performance measures in screening assays: signal window, Z′ factor, and assay variability ratio. J Biomol Screen 11, 247-52 (2006).

4. Ritz, C., Baty, F., Streibig, J.C. & Gerhard, D. Dose-Response Analysis Using R. Plos One 10(2015).

5. Zeileis, A. & Hothorn, T. Diagnostic Checking in Regression Relationships. R News 2, 7-10 (2002).

6. Zeileis, A. Econometric Computing with HC and HAC Covariance Matrix Estimators. Journal of Statistical Software 1, 1-17 (2004).

1. A method for treating a patient presenting with one or more of chronic inflammatory injury, metaplasia, dysplasia or cancer of an epithelial tissue, which method comprises administering to the patient an agent that selectively kills or inhibits the proliferation or differentiation of pathogenic epithelial stem cells (PESC) in the epithelial tissue relative to normal stem cells of the epithelial tissue.

2. A method of reducing proliferation, survival, migration, or colony formation ability of a pathogenic epithelial stem cell (PESC) in a subject in need thereof comprising contacting the cell with a therapeutically effective amount of an agent that selectively kills or inhibits the proliferation or differentiation of PESC relative to normal epithelial stem cells.

3. A pharmaceutical preparation for treating one or more of chronic inflammatory injury, metaplasia, dysplasia or cancer of an epithelial tissue, which preparation comprises an agent that selectively kills or inhibits the proliferation or differentiation of pathogenic epithelial stem cells (PESC) in the epithelial tissue relative to normal stem cells of the epithelial tissue.

4. A drug eluting device for treating one or more of chronic inflammatory injury, metaplasia, dysplasia or cancer of an epithelial tissue, which device comprises drug release means including an agent that selectively kills or inhibits the proliferation or differentiation of pathogenic epithelial stem cells (PESC) in the epithelial tissue relative to normal stem cells of the epithelial tissue, which device when deployed in a patient positions the drug release means proximal to the surface of the diseased tissue and releases the agent in an amount sufficient to achieve a therapeutically effective exposure of the diseased tissue to the agent.

5. A method for treating a patient presenting with one or more of esophagitis, Barrett's esophagus, esophageal dysplasia or esophageal cancer, which method comprises administering to the patient an agent that selectively kills or inhibits the proliferation or differentiation of Barrett's Esophagus stem cells (BESC) relative to normal esophageal stem cells.

6. A method of reducing proliferation, survival, migration, or colony formation ability of a Barrett's Esophagus stem cell (BESC) in a subject in need thereof comprising contacting the cell with a therapeutically effective amount of an agent that selectively kills or inhibits the proliferation or differentiation of BESC relative to normal esophageal stem cells.

7. A pharmaceutical preparation for treating one or more of esophagitis, Barrett's esophagus, esophageal dysplasia or esophageal cancer, which preparation comprises an agent that selectively kills or inhibits the proliferation or differentiation of Barrett's Esophagus stem cells (BESC) relative to normal esophageal stem cells.

8. A drug eluting device for treating one or more of esophagitis, Barrett's esophagus, esophageal dysplasia or esophageal cancer, which device comprises drug release means including an agent that selectively kills or inhibits the proliferation or differentiation of Barrett's Esophagus stem cells (BESC) relative to normal esophageal stem cells, which device when deployed in a patient positions the drug release means proximal to the luminal surface of the esophagus and releases the agent in an amount sufficient to achieve a therapeutically effective exposure of the luminal surface to the agent.

9. The method of claim 5, for the treatment of Barrett's Esophagus.

10. The method of claim 5, for the treatment of esophageal adenocarcinoma.

11. The method of claim 5, wherein the agent is administered during or after endoscopic ablation therapy, such as radiofrequency ablation, photodynamic therapy or cryoablation of esophageal tissue.

12. The method of claim 5, wherein the agent is administered by submucosal injection of esophageal tissue.

13. The preparation of claim 7, wherein the agent is formulated for submucosal injection of esophageal tissue.

14. The method of claim 5, wherein the agent is formulated as part of a bioadhesive formulation.

15. The method of claim 5, wherein the agent is formulated as part of a drug-eluting particle, drug eluting matrix or drug-eluting gel.

16. The method of claim 5, wherein the agent is administered by topical application to the epithelial tissue.

17. The preparation of claim 3, wherein the agent is formulated for topical application to epithelial tissue.

18. The method of claim 16, wherein the agent is formulated as part of a bioadhesive formulation.

19. The method of claim 16, wherein the agent is formulated as part of a drug-eluting particle, drug eluting matrix or drug-eluting gel.

20. The method of claim 5, wherein the agent is co-administered with an analgesic, an anti-infective or both.

21. The preparation of claim 3, wherein the agent is co-formulated with an analgesic, an anti-infective or both.

22. The preparation of claim 3, wherein the agent is formulated as a liquid for oral delivery to the epithelial tissue, such as the esophagus.

23. The preparation of claim 3, wherein the agent is formulated as a single oral dose.

24. The device of claim 4, wherein the drug eluting device is a drug eluting stent.

25. The device of claim 4, wherein the drug eluting device is a balloon catheter having a surface coating including the agent.

26. The method of claim 5, wherein the agent selectively inhibits the proliferation or differentiation of PESCs, or selectively kills PESCs, with an IC₅₀ that is ⅕^{th} or less the IC₅₀ for normal epithelial stem cells in the same tissue, more preferably 1/10^{th}, 1/20^{th}, 1/50^{th}, 1/100^{th}, 1/250^{th}, 1/500^{th} or even 1/1000^{th}.

27. The method of claim 5, wherein the agent inhibits the proliferation or differentiation of PESCs, or kills PESCs, with an IC₅₀ of 10⁻⁶ M or less, more preferably 10⁻⁷ M or less, 10⁻⁸ M or less or 10⁻⁹ M or less.

28. The method of claim 5, wherein the agent is cell permeable, such as characterized by a permeability coefficient of 10⁻⁹ or less, more preferably 10⁻⁸ or less or 10⁻⁷ or less.

29. The method of claim 5, wherein the agent is a histone demethylase inhibitor.

30. The method of claim 5, wherein the agent is a JmjC inhibitor.

31. The method of claim 30, wherein the JmjC inhibitor binds to and inhibits a catalytic JmjC domain.

32. The method of claim 30, wherein the JmjC inhibitor is a plant homodomain (PHD) inhibitor or a protein-protein interaction inhibitor.

33. The method of claim 30, wherein the JmjC inhibitor is a pan-JmjC demethylase inhibitor.

34. The method of claim 30, wherein the JmjC inhibitor is JIB04.

35. The method of claim 5, wherein the agent is a receptor tyrosine kinase inhibitor.

36. The method of claim 35, wherein the receptor tyrosine kinase inhibitor is an EGFR inhibitor, a HER2 inhibitor or a dual EGFR/HER2 inhibitor.

37. The method of claim 5, wherein the agent is a proteasome inhibitor.

38. The method of claim 5, wherein the agent is a STAT inhibitor, preferably a STAT3 inhibitor.

39. The method of claim 5, wherein the agent is a FLT3 inhibitor.

40. The method of claim 5, wherein the agent is a GSK3 inhibitor.

41. The method of claim 5, wherein the agent is a HSP90 inhibitor, a HSP70 inhibitor or a dual HSP90/HSP70 inhibitor.

42. The method of claim 5, wherein the agent is a selected from the group consisting of:

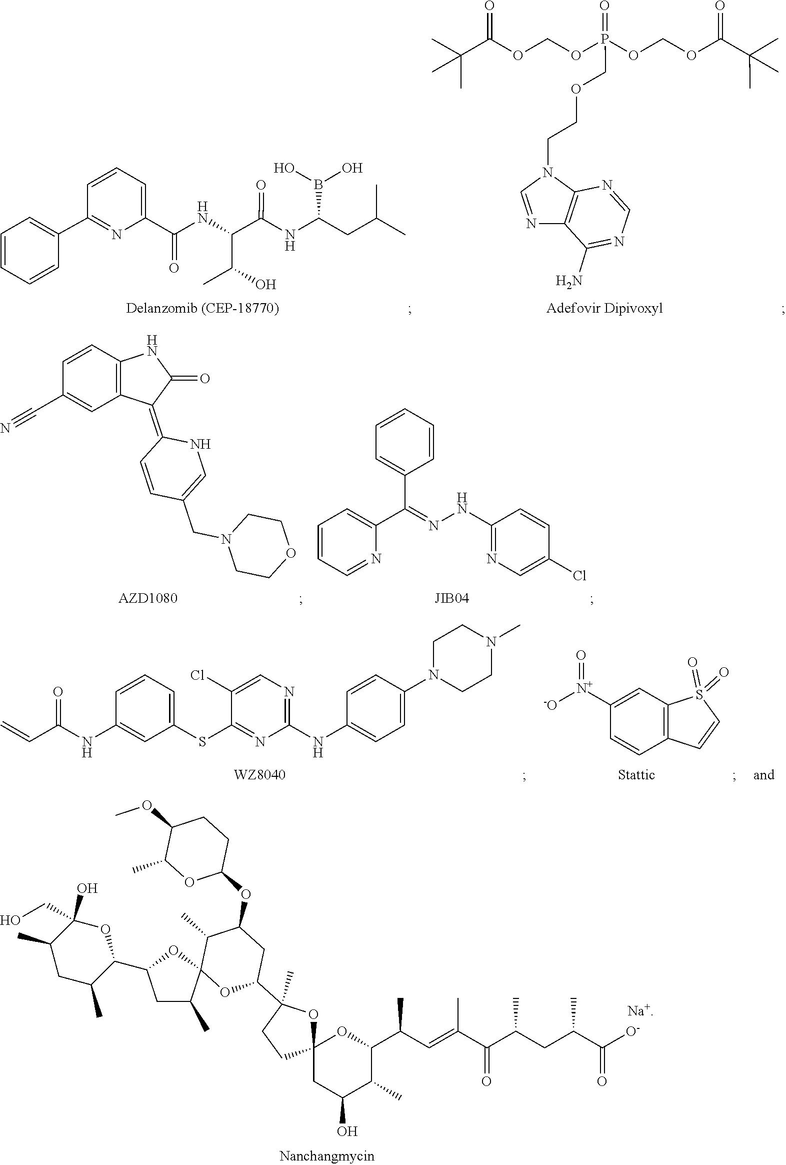

43. The method of claim 5, further comprising combining the agent with a second drug agent that selectively promotes proliferation of normal epithelial stem cells in the target with an EC₅₀ at least 5 times more potent than for PESCs in the target tissue, more preferably with an EC₅₀ 10 times, 50 times, 100 times or even 1000 times more potent than for PESCs.

44. The method of claim 5, wherein the second drug agent promotes proliferation of normal esophageal stem cells with an EC₅₀ of 10⁻⁶ M or less, more preferably 10⁻⁷ M or less, 10⁻⁸ M or less or 10⁻⁹ M or less.

45. The method of claim 5, wherein the second drug agent is pan-inhibitor of ABL kinase inhibitor, preferably a BCR-ABL kinase inhibitor.

46. The method of claim 45, wherein the second drug agent is a pan-inhibitor selected from the group consisting of imatinib, nilotinib, dasatinib, bosutinib and ponatinib or pharmaceutically acceptable salt(s), solvate(s), and/or hydrate(s) thereof, and is preferably ponatinib or pharmaceutically acceptable salt(s), solvate(s), and/or hydrate(s) thereof.

47. The method of claim 5, wherein the second drug agent is FLT kinase inhibitor, preferably a FLT3 kinase inhibitor.

48. The method of claim 47, wherein the second drug agent is a FLT3 inhibitor selected from the group consisting of quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof, and is preferably quizartinib or pharmaceutically acceptable salt(s), solvate(s), and/or hydrate(s) thereof.

49. The method of claim 5, further comprising combining the agent with a one or more antitussives, antihistamines, antipyretics, analgesics, anti-infective agents and/or chemotherapeutic agents.

50. The method of claim 43, wherein the agent and the second agent are administered to the patient as separate formulations.

51. The method of claim 43, wherein the agent and the second agent are co-formulated together.

52. The method of claim 49, wherein the agent and the one or more antitussives, antihistamines, antipyretics, analgesics, anti-infective agents and/or chemotherapeutic agents are co-formulated together.

53. The method of claim 5, wherein the patient is a human patient.

54. A bioadhesive nanoparticle having a polymeric surface with an adhesive force equivalent to an adhesive force of between 10 N/m² and 100,000 N/m² measured on human mucosal surfaces, which nanoparticle further includes an anti-PESC Agent dispersed therein or thereon, wherein the nanoparticle elutes the anti-PESC Agent into the mucous gel layer when adhered to mucosal tissue.

55. A submucosal retentive formulation comprising an anti-PESC Agent and one or more pharmaceutically acceptable excipients, which formulation is injectable submucosally and forms a submucusal depot releasing an effective amount of the an anti-PESC Agent to the tissue the site of injection.

56. An injectable thermogel for submucosal injection, comprising an anti-PESC Agent and optionally one or more pharmaceutically acceptable excipients, wherein the thermogel has a low-viscosity fluid at room temperature (and easily injected), and becomes a non-flowing gel at body temperature after injection.

57. A drug eluting device comprising drug release means including an anti-PESC Agent, which device when deployed in a patient positions the drug release means proximal to a target epithelial tissue to be treated and releases the anti-PESC Agent in an amount sufficient to achieve a therapeutically effective exposure of the target tissue to the anti-PESC Agent.

58. Single oral dosage formulation comprising an anti-PESC Agent, an ESO Regenerative Agent, and a pharmaceutically acceptable excipient, which single oral dosage formulation taken by an adult patient produces a concentration of the anti-PESC Agent and the ESO Regenerative Agent in esophageal tissue effective to slow or reverse the progress of an esophageal metaplasia, dysplasia, cancer or a combination thereof.


SEQUENCE LISTING (2 sequences)

SEQ ID NO: 1 - DNA, 63 bp, Artificial sequence
  Synthetic nucleic acid
cagggaagtttcgagaagtcctatagtgaagccacagatgtataggactctcgaacttccctt

SEQ ID NO: 2 - DNA, 63 bp, Artificial sequence
  Synthetic nucleic acid
acaccagcagtagcaacagcaatagtgaagccacagatgtattgctgttgctactgctggtgg